SEQUENCE LISTING (43 sequences)

SEQ ID NO: 1 - protein, 414 aa, Mus musculus
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGNPGGFGNQGGFGNSRGGGAGLGNNQGGNMGGGMNFGAFSINPAMMAAAQAALQSSWGMMGMLASQQNQSGPSGNNQSQGSMQREPNQAFGSGNNSYSGSNSGAPLGWGSASNAGSGSGFNGGFGSSMDSKSSGWGM

SEQ ID NO: 2 - DNA, 7454 bp, Mus musculus
ctcggaaggccgagtggccgttctgtccttcatctgtcagtttttcagacccagctgttttcattgttgcgtttctttacttttttctatacgccgaagagcctgctagcatccgagcctctgggaggagagagcgcctgtggcttccctcggagagcgcccctcctgcagggaagccagtgggagaggccgaaggcgggcgagggcgggaggcggccctagcgccattttgtgggcacggagcggtagcgcggctgttgtcggattccttcccgtctgtgcttcctccttgtgcttcctagcagtggcctagcggagatttaagcaaagatgtctgaatatattcgggtaacagaagatgagaacgatgaacccattgaaataccatcagaagacgatgggacggtgttgctgtccacagttacagcccagtttccaggggcatgcggcctgcgctaccggaatcccgtgtctcagtgtatgagaggagtccgactggtggaaggaattctgcatgccccagatgctggctggggcaatctggtatatgttgtcaactatcccaaagataacaaaaggaaaatggatgagacagatgcttcctctgcagtgaaagtgaaaagagcagtccagaaaacatctgacctcatagtgttgggtctcccctggaaaacaactgagcaggatctgaaagactatttcagtacttttggagaggttcttatggttcaggtcaagaaagatcttaaaactggtcactcgaaagggtttggctttgttcgatttacagaatatgaaacccaagtgaaagtaatgtcacaacgacatatgatagatgggcgatggtgtgactgtaaacttcccaactctaagcaaagcccagacgagcctttgagaagcagaaaggtgtttgttggacgttgtacagaggacatgactgctgaagagcttcagcagtttttctgtcagtatggagaagtggtagatgtcttcattcccaaaccattcagagcttttgccttcgtcacctttgcagatgataaggttgcccagtctctttgtggagaggatttgatcattaaaggaatcagcgtgcatatatccaatgctgaacctaagcataatagcaatagacagttagaaagaagtggaagatttggtggtaatccaggtggctttgggaatcagggtgggtttggtaacagtagagggggtggagctggcttgggaaataaccagggtggtaatatgggtggagggatgaactttggtgcttttagcattaacccagcgatgatggctgcggctcaggcagcgttgcagagcagttggggtatgatgggcatgttagccagccagcagaaccagtcgggcccatctgggaataaccaaagccagggcagcatgcagagggaaccaaatcaggcttttggttctggaaataattcctacagtggttctaattctggtgccccccttggttgggggtcagcatcaaatgcaggatcgggcagtggttttaatgggggctttggctcgagcatggattctaagtcttctggctggggaatgtaggtggtggggggtggttagtaggttggttattaggttaggtagatttagaatggtgggattcaaatttttctaaactcatggtaagtatattgtaaaatacatatgtactaaaattttcagattggtttgttcagtgtggagtatattcagcagtatttttgacatttttctttagaaaaaaagaggggaaagctaaatgaattttataagttttgttatataaagggttaaaatactgagtgggtgaaagtgaactgctgtttgcctaattggtaaaccaacactacaattgatctcagaaggtttctctgtaatattctatcattgaaattgttaatgaattctttgcatgttcagagtagaaaccattggttagaactacattcttttctccttattttaatttgaatcccaccctatgaattttttccttaggaaaatctccatttgggagatcatgatgtcatggtgtttgattcttttggttttgtttttaacacttgtcttccttcatatacgaaagtacaatatgaagccttcatttaatctctgcagttcatctcatttcaaatgtttatggaagaagcacttcattgaaagtagtgctgtaaatattctgccataggaatacttctgtctacatgctttctcatccaagaattcgtcatcacgctgcacaggctgcgtctttgacggtgggtgttccatttttatccgctactctttatttcatggagtcgtatcaacgctatgaacgcaaggctgtgatatggaaccagaaggctgtttgaacttttgaaaccttgtgtgggattgatggtggtgccgaggcatgaaaggctagtatgagcgagaaaaggagagcgcgtgcagagacttggtggtggaaaatggatattttttaacttggagagatgtgtcactcaatcctgtggctttggtgagagagtgtgcagagagcaatgatagcaaataacgtacgaatgttttacatcaaaggacatccacatcagttggaagactttgagttttgttcttaggaaacccactttagttgaatgtgttaagtgaaatacttgtacttccctccccctctgtcaactgctgtgaatgctgtatggtgtgtgttctcctctgttactgatctggaagtgtgggaacgtgaactgaagctgatgggctgcgaacatggactgagcttgtggtgtgctttgcaggagaacttggaagcagagttcaccagtgagctcaggtgtctcaaagaagggtggaagttctcatgtctgttagctattcataagaatgctgtttgctgcagttctgtgtcctgtgcttggatgcttttttataagagttgtcattgttggaaattcttaaataaaactgatttaaataatatgtgtctttgttttgcagccctgaatgcaaagaattcatagcagttaattcccctttttgacccttttgagatggaactttcataaagtttcttggcagtagtttattttgcttcaaataaacttatttgaaaagttgtctcaagtcaaatggattcatcacctgtcatgcattgacacctgatacccagacttaattgctatttgttcttgcattgtccaaagtgaaagtttttctttggtttgtttttaatttagtttttcttaagtctgggtgaccgcacctaaaatggtaagcagttaccctctggcttgttctgagtgcctctgtgcatttgattttctatttacatgctgtataaatctccactggggaatcatgccttctaaaaatatttgggagagggcaaaagagttgatttctaatgctttgtagcagagcatatcaatgggaaagaaggttaagcacctttctgtttgggatttgaaaagtggaattaattgcaatagggatgaagtagaagaaaccaagaaaccatgtgcctgaaatacattaagaagcctgattgatagctttaagaactagtagggtgggttgtcttacctgtggcagtcttaagtgaggtaggcttttgccctcctgaatgtgggggttatgtagtgatgaatatgctcacaaaatcagattagactgtcaatgcattgttaatgtaaaagcaataatacattgattattgtacttttcctgtaactactgagaccggaggcgctccttttctaactggaagaatgggacagtttttgtgttggtagtttttcctaatgcccttacctaaatagattatgataaataggtttgtcattttgcaagttgcgtgttttaaaattttatatccgttagagacttgttatgaacacattgtttcattatacagtatcctctgtaaaaggatcgtgagttattgtaagtttttttctctgcatctaaccctgcatgatttccaaaccctgtgcatctgaattttgcattttagcactgtttgcactgttactcagcagcagtaacatggtaacattaaaatggttttcggggacctccaaagacggccaggagtcctggggtaagttacttgtcaatggcatggttttgatcccttttttacacttgttaaagacttactggtcatagaagtctttcagtgttgatcagccttttaacatgtttatggatgacatagctgtagttagttacttgccgtaaatgaggttttagaaataaactacttggcaaagatttggttttgaaagtctggtcatcaaaacgcgttcattccttagaaataatgaagaacaactctttgaaccacagttgaataaaaggttttcttgccaccaacagtttagtgtctggagtcttactggaagaaaaaaaaattctatatcatgacaatgctagaaaagttaaggtgacttatgtgggaagatgcaatatagcattttcatcctttaaaatttgagtctccaggtgggtgtggtggcccatgcttttaatcccagaattggtgtaaatgagttgtaggccagctatttccccatcttgaggcaccctgtcttgccttgttggaagagccagttaaaatcaaacatgacctttaaggtcagcatcttagcagaagagcagtttatttcaggataacttactgtttttgatacataagcaaatgactgtaccttgtacagttacggttgacttccctgagcccaacgctcacctaagaaaagtgggctgggtatagtgaaacacctgttaaggtttttggaatgatttgctaaattgcccttgtaaagggtaaaatgctgtttcgtgttctttttatctgacaatttggtgaatctggtagaacatgcctatatcccaatattctggaatggacttggtgttaatttaatagctgatctaatgtgaaggtcacacacctgctttccgcttaccttccaaaaggtattctggaaccactcagaagttactcagaaagtaagagcactttctgagctcattaagaccaaatctacacactagacacaacaagccctttgttggccagaaatggaaacagccagtataaaataagtagattgtagggaatcaaatacaacttgttttttctgtgttgggggttggccaagcactgttaaacacaaatcaaagctgatattggcaagtgtttggacctgtaacaatctcacctgctctgattttggggtaggctgtcattcttaggtttgttactaagcttcccaggtacttggcgatatgaggaacaatgattggacgatgcaaattagaaattacttattatgttctcaatccagggaatatagttgatgacttttgtgtagaccccatactggtctgccgccccacaattaatggaaccccaggaaactattcctcccacaaaccatcgctgtgtctcattatctagaaacactaatgcccccccactgtcacctctgcagctgtccttgccaccagtctctaagccagcacagagcatgttagcgcttactcttactcctggatagagcttttcatacacggcggtacatttttggtggtcagcaattggtatgtccacaaacattaggtttctagcaagaagccccttctgggttaacccccagccagccacagttccagtgaagtctgttctcattaaggatgcagcttcttttcgcggtaggcaaacaggcatgatgcttccgttgattgtgactttgttcttgagtttaatcaatgctatatcattgtcaaaaccagcaccgtgagtgtagccttcatgtataaagatttcctcgggccaggcttgagtgtaatgaggtgagagccttttgaggatgcccattcggatgttcagggaggacgctgccattcttttctcatatacagcatgagcggctgttaggacccaattgtcatgtataagtgcacctgctgctgctgtagtttgacccagcaacaagacttgccaaggaaagtcaccaggctttgcaggctgccctccaactatgcgtcctcctatagtgtgtgtggacagcccacaaactgagggataaaaaacaagtatttaatgccctacaaattgataggcatcctccctgttgagtgaggctatttaagtttttgtttagcctgctcacctccttgtaaagatcagtaagagatctcagagattgttttgctgaaagagaacagcatgagggagtctggacaggacttgtgtgcaggaggacatgacactacttagaggccaaagacaaccccctcaccacaccctgagctgcttgtttttctctttttgggctctctgggaattctgggaaagcaggaactatgatttacaaagtttctgttgtctttaaatgtaagactctaaattacaatgttgcagcaatagccaaagtgcttctggttcaaaaattggtaattttggtctggtggagccctccaaacacttttaccgttctttgcaaactgagggctcaggaatgcaacacatgttctttattgtggttgtgcactttgattaaaacttggaagccgcatgtcagccaaatacaaggctagaaaactaatttaaaccagctaacacgggggtaatgagtgtattattacctttcaattaaaaaaaaagcactctcaactgttgttggagccaattctggtaaagaaatttaagtactattaaaaggcaaattgcattaatgtttaaaaatcttgatgtcgttgaaaacaattgcttagggaaataatgaagttattagctttggggtttaatagcatttttacagagaagaaaagtaacaagagttcttggttataaatgtataaacggtttgagataatttaagaaatcatttaattttttatgcttgcctagttataaggtcaaaaacaatcaagtgcatgatgcacctagcttccgtgtggaaggggaaatgtgagcacactgttgggaaacactaagctccagcctcagccaagtgctgagctttctgcctccccagccagaccctgcctattgtctgccagctactctgtcagctatgaatctcttttataaatggcgtccattaccaggctcacaaaccggggggagtttttctcctttggagctcgtccagaatccatcagcctcacacacatatttacctgcaagtcattggaaaagcaaaaatgtttagctgtagttgtcatttgcttgaataaccccttgaaaaatgttgattcttgagcatctgtggtggggagaggtgtgtgaataaccattttacatgatttcataaataggtgtctgcattaccatgtttgcttgcaaagtggaaaccttttagatgtgtaacttgaatatgtatcaagatctcaagtgcttaatgataaggttttgacttgttaaattaaaccatttggaatatattgtg

SEQ ID NO: 3 - protein, 285 aa, Rattus norvegicus
MSEYIRVTEDENDEPIEIPSEDDGTVLLSTVTAQFPGACGLRYRNPVSQCMRGVRLVEGILHAPDAGWGNLVYVVNYPKDNKRKMDEADASSAVKVKRAVQKTSDLIVLGLPWKTTEQDLKDYFSTFGEVLMVQVKKDLKTGHSKGFGFVRFTEYETQVKVMSQRHMIDGRWCDCKLPNSKQSPDEPLRSRKVFVGRCTEDMTAEELQQFFCQYGEVVDVFIPKPFRAFAFVTFADDKVAQSLCGEDLIIKGISVHISNAEPKHNSNRQLERSGRFGGKSPFGRS

SEQ ID NO: 4 - DNA, 2040 bp, Rattus norvegicus
ttttgtgggcacgaagcggtagctcggctgttgttgggttcctttccgtcttcgatccttcgttgtgcttcctagcagcggcccagtggagatttaagcaaagatgtctgaatatattcgggtaacagaagatgagaatgatgagcccattgaaataccatcagaagacgatgggacagtgttgctgtccacagttacagcccagtttccaggggcgtgtggcctgcgctaccggaatccagtgtctcagtgtatgagaggtgtccgactggtggaaggaattctgcatgccccagatgctggctggggcaatctggtctatgttgtcaactatcccaaagataacaaaaggaaaatggatgaggcggatgcttcctctgcagtgaaagtgaaaagagcagtccagaagacatctgacctcatagtgttgggtctcccctggaaaacaacagagcaggacctaaaagactacttcagtacttttggagaggttcttatggttcaggtcaagaaagatcttaaaactggtcactcaaaagggtttggctttgttcgatttacggaatatgaaactcaagtgaaagtaatgtcacagcgacatatgatagatgggcgatggtgtgactgtaaacttccaaattctaagcaaagcccagacgagcctttgagaagcagaaaggtgtttgttggacgttgtacagaggacatgactgctgaagagcttcagcagttcttctgtcagtatggagaagtggtagatgtcttcattcccaaaccattcagagcttttgcctttgttacctttgcagatgataaggttgcccagtctctttgtggagaggacttgatcattaaaggaatcagcgtgcatatatccaatgctgaacctaagcataatagcaatagacagttagaaagaagtggaagatttggtggaaaatctccatttgggagatcatgatgtcatggtgtttggttcttttggttttgtttttaacacttgtcttccttcatatacgaaagtacaatatgaagccttcatttaatctctgcagttcatctcatttcaaatgtttatggaagaagcacttcattgaaagtagtgctgtaaatattctgccataggaatacttctgtctacatgctttctcatccaagaattcgtcatcacgctgcacaggctgcgtctttgacggtgggtgttccatttttatccgctactctttatttcatggaatcgtatcaacgctatgaacgcaaggctgtgatatggaaccagaaggctgtttgaacttttgaaaccttgtgtgggattgatggtggtgccgaggcatgaaaggctagtatgagcgagaaaaggagagagcgcgtgcagagacttggtggtggaaaatggatattttttaacttggagagatgtgtccctcaatcctgtggctttggtgcgagagtgtgcagagagcaatgatagcagataactaacgtacgagtgtttctgcatcagaggacatccacgtctgttggaagactttgagttttgttcttaggaaacccacagtagctgaatgtgttaagtgaaatacttgtacttccctcccctctgtcaactgctgtgaatgctgtatggtgtgtgttctcctctgttactgatctggaagtgtgggaacgtgaactgaagctgatgggctgcgaacatggactgagcttgtggtgtgctttgcaggagaacttggaagcagagttcaccagtgagctcaggtgtctcaaagaagggtggaagttctcatgtctgttagctattcataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattcccctttttgacccttttgagatggaactttcataaagtttcttggcagtagtttattttgcttcaaataaacttatttgaaaagtt

SEQ ID NO: 5 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 6 - DNA, 4185 bp, Homo sapiens
attttgtgggagcgaagcggtggctgggctgcgcttgggtccgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccaca

SEQ ID NO: 7 - protein, 6 aa, Artificial Sequence
  RRM1 RNP2 consensus sequence
LIVLGL

SEQ ID NO: 8 - protein, 8 aa, Artificial Sequence
  RRM1 RNP1 consensus sequence
KGFGFVRF

SEQ ID NO: 9 - protein, 6 aa, Artificial Sequence
  RRM2 RNP2 consensus sequence
VFVGRC

SEQ ID NO: 10 - protein, 7 aa, Artificial Sequence
  RRM2 RNP1 consensus sequence
RAFAFVT

SEQ ID NO: 11 - DNA, 22 bp, Artificial Sequence
  TDP-43 Ex3-Ex4 assay Forward Primer
tgtgactgtaaacttcccaact

SEQ ID NO: 12 - DNA, 21 bp, Artificial Sequence
  TDP-43 Ex3-Ex4 assay Reverse Primer
ctcttcagcagtcatgtcctc

SEQ ID NO: 13 - DNA, 24 bp, Artificial Sequence
  TDP-43 Ex3-Ex4 Probe
aagcccagacgagcctttgagaag

SEQ ID NO: 14 - DNA, 22 bp, Artificial Sequence
  Crem Ex1-Ex2 assay Forward Primer
tggctgtaactggagatgaaac

SEQ ID NO: 15 - DNA, 21 bp, Artificial Sequence
  Crem Ex1-Ex2 assay Reverse Primer
ccttgtggcaaagcagtagta

SEQ ID NO: 16 - DNA, 25 bp, Artificial Sequence
  Crem Ex1-Ex2 Probe
acatgccaacttaccagatccgagc

SEQ ID NO: 17 - DNA, 22 bp, Artificial Sequence
  Crem Ex1-Cryptic assay Forward Primer
tggctgtaactggagatgaaac

SEQ ID NO: 18 - DNA, 22 bp, Artificial Sequence
  Crem Ex1-Cryptic assay Reverse Primer
ggaagagaagcaactcctcaaa

SEQ ID NO: 19 - DNA, 24 bp, Artificial Sequence
  Crem Ex1-Cryptic Probe
acacacacacacacacacacacac

SEQ ID NO: 20 - DNA, 22 bp, Artificial Sequence
  Crem Cryptic-Ex2 assay Forward Primer
catgggttccaaaggatcaaac

SEQ ID NO: 21 - DNA, 20 bp, Artificial Sequence
  Crem Cryptic-Ex2 assay Reverse Primer
tgtggcaaagcagtagtagg

SEQ ID NO: 22 - DNA, 25 bp, Artificial Sequence
  Crem Cryptic-Ex2 Probe
acatgccaacttaccagatccgagc

SEQ ID NO: 23 - DNA, 20 bp, Artificial Sequence
  Fyxd2Ex3-Ex4 assay Forward Primer
actatgaaaccgtccgcaaa

SEQ ID NO: 24 - DNA, 17 bp, Artificial Sequence
  Fyxed Ex3-Ex4 assay Reverse Primer
cccacagcggaaccttt

SEQ ID NO: 25 - DNA, 24 bp, Artificial Sequence
  Fyxed Ex3-Ex4 Probe
cgtgggcctcctcatcattctcag

SEQ ID NO: 26 - DNA, 20 bp, Artificial Sequence
  Fyxed Ex3-Cryptic assay Forward Primer
actatgaaaccgtccgcaaa

SEQ ID NO: 27 - DNA, 22 bp, Artificial Sequence
  Fyxed Ex3-Cryptic assay Reverse Primer
cctctttgcttcaccaaatgtc

SEQ ID NO: 28 - DNA, 24 bp, Artificial Sequence
  Fyxed Ex3-Cryptic Probe
cgtgggcctcctcatcattctcag

SEQ ID NO: 29 - DNA, 21 bp, Artificial Sequence
  Fyxed Cryptic-Ex4 assay Forward Primer
ttctggaattcccacacactc

SEQ ID NO: 30 - DNA, 17 bp, Artificial Sequence
  Fyxed Cryptic-Ex4assay Reverse Primer
cccacagcggaaccttt

SEQ ID NO: 31 - DNA, 26 bp, Artificial Sequence
  Fyxed Cryptic-Ex4 Probe
ctctgaatgaaagctgggctcttgga

SEQ ID NO: 32 - DNA, 17 bp, Artificial Sequence
  Crlf1 Ex1-Ex2 assay Forward Primer
ctgtcctcgctgtggtc

SEQ ID NO: 33 - DNA, 18 bp, Artificial Sequence
  Crlf1 Ex1-Ex2 assay Reverse Primer
ggaggagccgatgagaag

SEQ ID NO: 34 - DNA, 22 bp, Artificial Sequence
  Crlf1 Ex1-Ex2 Probe
tctgttgctctgtgtcctcggg

SEQ ID NO: 35 - DNA, 18 bp, Artificial Sequence
  Crlf1 Ex1-Cryptic assay Forward Primer
gtcgcctctgttgctctg

SEQ ID NO: 36 - DNA, 22 bp, Artificial Sequence
  Crlf1 Ex1-Cryptic assay Reverse Primer
tccatccattcatccatccatc

SEQ ID NO: 37 - DNA, 21 bp, Artificial Sequence
  Crlf1 Ex1-Cryptic Probe
acctcagttcctggcatattg

SEQ ID NO: 38 - DNA, 22 bp, Artificial Sequence
  Crlf1 Cryptic-Ex2 assay Forward Primer
gagacctcagagaactgaatgg

SEQ ID NO: 39 - DNA, 22 bp, Artificial Sequence
  Crlf1 Cryptic-Ex2 assay Reverse Primer
ccaggtgtgtctccatgtatag

SEQ ID NO: 40 - DNA, 24 bp, Artificial Sequence
  Crlf1 Cryptic-Ex2 Probe
ttctcatcggctcctccctgcaag

SEQ ID NO: 41 - DNA, 26 bp, Artificial Sequence
  TDP-43 Ex6-Ex7 assay Forward Primer
gctgaacctaagcataatagcaatag

SEQ ID NO: 42 - DNA, 22 bp, Artificial Sequence
  TDP-43 Ex6-Ex7 assay Reverse Primer
ggatgagaaagcatgtagacag

SEQ ID NO: 43 - DNA, 29 bp, Artificial Sequence
  TDP-43 Ex6-Ex7 Probe
tggaagaagcacttcattgaaagtagtgc

(19) United States
(12) Patent Application Publication
(10) Pub. No.: US 2020/0404890 A1
(43) Pub. Date: Dec. 31, 2020

(54) MODELING TDP-43 PROTEINOPATHY

(71) Applicant: Regeneron Pharmaceuticals, Inc., Tarrytown, NY (US)

(72) Inventors: Aarti Sharma-Kanning, New York, NY (US); David Frendewey, New York, NY (US); Brian Zambrowicz, Sleepy Hollow, NY (US)

(21) Appl. No.: 16/913,729

(22) Filed: Jun. 26, 2020

Related U.S. Application Data
(60) Provisional application No. 62/867,785, filed on Jun. 27, 2019.

(51) Int. Cl. A01K 67/027; C12N 15/85; C07K 14/47; A61K 49/00; C12N 15/113; C12N 15/11; C12N 9/22

(52) U.S. Cl. CPC A01K 67/0276; C12N 15/8509; C07K 14/4703; A61K 49/0008; C12N 15/113; C12N 15/11; C12N 2310/20; C12N 2015/8536; A01K 2217/077; A01K 2267/0318; C12N 2310/11; C12N 2310/141; C12N 9/22

(57) ABSTRACT

Described herein is the discovery that neither the nuclear localization signal (NLS) nor the prion-like domain (PLD) of TDP-43 is necessary for embryonic stem cell culture and differentiation into motor neurons in vitro. The ability of ES cells to express these TDP-43 mutants and differentiate into motor neurons that exhibit an ALS-like phenotype whereby the TDP-43 mutants redistribute to and aggregate in the cytoplasm and fail to regulate cryptic exon splicing allows these cells to act as a model of TDP-43 proteinopathy for the testing of candidate therapeutic agents that may resolve such proteinopathy. Additionally, these ES cells may be used to successfully generate non-human animals, e.g., mice, that also exhibit hallmark symptoms of ALS and that may be used in testing candidate agents useful in treating TDP-43 proteinopathies.

### CROSS-REFERENCE TO RELATED APPLICATIONS

This application claims the benefit under 35 U.S.C. § 119(3) of U.S. Provisional Application Ser. No. 62/867,785 (filed Jun. 27, 2019) the disclosure of which is hereby incorporated by reference in its entirety.

### REFERENCE TO A SEQUENCE LISTING SUBMITTED AS A TEXT FILE VIA EFS WEB

The Sequence Listing written in file 10312US01_ST25.txt is 35 kilobytes, was created on Jun. 26, 2020, and is hereby incorporated by reference.

### TECHNICAL FIELD

Described herein are methods of evaluating the biological role(s) of TDP-43 and its domains, non-human animals and non-human animal cells for same, and nucleic acids for same. Models of TDP-43 proteinopathies comprising such non-human animals, non-human animal cells or nucleic acids, and methods of using same, are also provided.

### BACKGROUND OF THE INVENTION

Amyotrophic lateral sclerosis (ALS) is a devastating neurodegenerative disease that affects motor neurons, causing limb paralysis and eventual death as the result of failure of the diaphragm muscle. A nearly universal pathological finding in postmortem examinations of ALS patient tissue is the accumulation of TDP-43 (transactive response DNA binding protein 43 kDa) in cytoplasmic inclusions.

TDP-43 is characterized as having a nuclear localization signal (NLS) domain, two RNA recognition motifs (RRM1 and RRM2), a putative nuclear export signal (NES) domain, and a glycine rich prion like domain (PLD). Similar to members of the heterogeneous nuclear ribonucleoprotein (hnRNP) family, TDP-43 is a predominantly nuclear RNA binding protein required for the viability of all mammalian cells and the normal development of animals. The redistribution of TDP-43 from the nucleus to the cytoplasm and its accumulation in insoluble aggregates are two key diagnostic hallmarks of ALS disease.

Although cytoplasmic accumulation of TDP-43 is associated with ALS, the relationship between each of the structural domains of TDP-43 and the biological function(s) of TDP-43 is not clear.

### SUMMARY OF THE INVENTION

Provided herein are embryonic stem (ES) cells, tissues cultured therefrom (e.g., primitive ectoderm, embryoid bodies, motor neurons), and non-human animals derived therefrom that express a mutant TDP-43 polypeptide lacking a functional structural domain and that may exhibit an ALS-like phenotype. Compositions and methods for making and using same are also provided. Mutated TARDBP genes encoding a mutant TDP-43 polypeptide lacking a functional structure domain and mutant TDP-43 polypeptides lacking a functional structural domain are also provided. Also provided are exemplary therapeutic oligonucleotides, e.g., antisense oligonucleotides, which may restore autoregulation of TARDBP expression.

Described herein are non-human animals (e.g., rodents (e.g., rat or mice)) and non-human animal cells (e.g., embryonic stem (ES) cells, embryoid bodies, embryonic stem cell derived motor neurons (ESMNs), etc.) comprising a mutated TARDBP gene that encodes a mutant TDP-43 polypeptide, e.g., wherein a mutated TARDBP gene comprises a nucleotide sequence of a wildtype TARDBP gene that comprises a mutation such that the mutant TDP-43 comprises an amino acid sequence of a corresponding wildtype TDP-43 polypeptide but for a mutation (e.g., one or more of a point mutation, a substitution, a replacement, an insertion, a deletion, etc.). In some embodiments, a wildtype TARDBP gene comprises a sequence set forth in SEQ ID NO:2 (including degenerate variants thereof), SEQ ID NO:4 (including degenerate variants thereof), or SEQ ID NO:6 (including degenerate variants thereof), which respectively encode a wildtype TDP-43 polypeptide comprising an amino acid sequence set forth as SEQ ID NO:1, SEQ ID NO:3, or SEQ ID NO:5.

In some embodiments, a mutated TARDBP gene that encodes a mutant TDP-43 polypeptide replaces an endogenous TARDBP gene at an endogenous TARDBP locus of the non-human animal or non-human animal cell. In some embodiments, the non-human animal cell or non-human animal is heterozygous for a mutated TARDBP gene that encodes a mutant TDP-43 polypeptide. For example, in some embodiments, a non-human animal or-human animal cell further comprises, in addition to a mutated TARDBP gene as described herein, (a) a wildtype TARDBP gene or (b) a TARDBP gene comprising a knockout mutation, e.g., a conditional knockout mutation. In some embodiments, the conditional knockout mutation comprises a site-specific recombination recognition sequence, e.g., a loxp sequence, optionally wherein the site-specific recombination recognition sequence (e.g., loxp sequence) flank a coding exon, e.g., exon 3. In some embodiments, the TARDBP gene comprising a knockout mutation comprises loxp sequences, which flank a deleted exon 3 of the TARDBP gene. In some embodiments, the knockout mutation comprises a deletion of the entire coding sequence of TDP-43 peptide.

In some embodiments, a non-human animal or non-human animal cell comprises (i) at an endogenous TARDBP locus, a replacement of an endogenous TARDBP gene with a mutated TARDBP gene that encodes a mutant TDP-43 polypeptide, and (ii) at an other endogenous TARDBP locus of a homologous chromosome, either the TARDBP gene comprising the knockout mutation or a wildtype TARDBP gene.

In some embodiments, a non-human animal or a non-human animal cell comprises at an endogenous TARDBP locus a TARDBP gene comprising a conditional knockout mutation and at an other endogenous TARDBP locus of a homologous chromosome a TARDBP gene comprising a deletion of the entire TARDBP coding sequence.

In some embodiments, a non-human animal cell or non-human animal is homozygous for the mutated TARDBP gene that encodes a mutant TDP-43 polypeptide.

In some embodiment, a non-human animal or non-human animal cell does not express a wildtype TDP-43 polypeptide.

In some embodiments, a non-human animal or non-human animal cell expresses a wildtype TDP-43 polypeptide.

In some embodiments, a non-human animal or non-human animal cell of any one of the preceding claims, comprises mRNA transcript levels of the mutated TARDBP gene that comparable to mRNA transcript levels of a wildtype TARDBP gene in a control cell, increased levels of the mutant TDP-43 polypeptide compared to levels of wildtype TDP-43 polypeptide in a control cell, a higher concentration of mutant TDP-43 polypeptide found in the cytoplasm than in the nucleus, e.g., of a motor neuron, mutant TDP-43 polypeptide with increased insolubility compared to a wildtype TDP-43 polypeptide cytoplasmic aggregates comprising the mutant TDP 43 polypeptide, increased splicing of cryptic exons, and/or decreased levels of the alternatively spliced TDP-43 form. In some embodiments, a non-human animal exhibits denervation of muscle tissue comprised of predominantly fast twitch muscles, such as anterior tibialis muscles and/or normal innervation of muscle tissues comprised of predominantly low twitch muscles, such as intercostal muscles.

In some embodiments, a non-human animal cell as described herein is cultured in vitro. Also described herein are non-human animal tissues comprising the non-human animal cells described herein.

In some embodiments, the non-human animal tissues and/or non-human animal cells are comprised in compositions.

In some embodiments, a mutant TDP-43 polypeptide lacks a functional structural domain compared to a wildtype TDP-43 polypeptide, and wherein the non-human animal or non-human animal cell expresses the mutant TDP-43 polypeptide, optionally wherein the wildtype TDP-43 polypeptide comprises a sequence set forth as SEQ ID NO:1, SEQ ID NO:3, or SEQ ID NO:5.

In some embodiments, a mutant TDP-43 polypeptide lacks a functional structural domain selected from the group consisting of the nuclear localization signal (NLS), the RNA recognition motif 1 (RRM1), the RNA recognition motif 2 (RRM2), the putative nuclear export signal (E), the prion like domain (PLD), or a combination thereof. In some embodiments, the mutated TARDBP gene is a TARDBP gene of the non-human animal that comprises a mutation, e.g., comprises a point mutation, a substitution, an insertion, a deletion, or a combination thereof. In some embodiments, a TARDBP gene of the non-human animal is set forth as SEQ ID NO:2 or SEQ ID NO:4. In some embodiments, the mutated TARDBP gene is a TARDBP gene of a human that comprises a mutation, e.g., a point mutation, a substitution, an insertion, a deletion, or a combination thereof. In some embodiments the mutated TARDBP. In some embodiments, the TARDBP gene of a human is set forth as SEQ ID NO:5.

In some embodiments, the mutant TDP 43 polypeptide lacks a functional structural domain due to one or more of the following (a) a point mutation of an amino acid in the NLS, (b) a point mutation of an amino acid in the RRM1, (c) a point mutation of an amino acid in the RRM2, (d) a deletion of at least a portion of the nuclear export signal, and (e) a deletion of at least a portion of the prion-like domain. For example, in some embodiments to mutant TDP-43 polypeptide comprises a sequence set forth as SEQ ID NO;1, SEQ ID NO:3, or SEQ ID NO:5 further comprising (a) a point mutation of an amino acid in the NLS, (b) a point mutation of an amino acid in the RRM1, (c) a point mutation of an amino acid in the RRM2, (d) a deletion of at least a portion of the nuclear export signal, and (e) a deletion of at least a portion of the prion-like domain. In some embodiments, (a) the point mutation of an amino acid in the NLS comprises K82A K83A, R84A, K95A, K97A, K98A, or a combination thereof, (b) the point mutation in RRM1 comprises F147L and/or F149L, (c) the point mutation in RRM2 comprises F194L and/or F229L, (d) the deletion of at least a portion of the nuclear export signal deletion comprises a deletion of the amino acids at and between positions 239 and 250 of a wildtype TDP-43 polypeptide, and (e) the deletion of at least a portion of the prion-like domain comprises a deletion of the amino acids at and between positions 274 and 414 of a wildtype TDP 43 polypeptide. In some embodiments, a mutant TDP-43 polypeptide comprises K82A K83A, R84A, K95A, K97A, and/or K98A compared to a wildtype TDP-43 polypeptide, optionally wherein the wildtype TDP-43 polypeptide comprises a sequence set forth as SEQ ID NO:1, SEQ ID NO:3, or SEQ ID NO:5. In some embodiments, a mutant TDP-43 polypeptide lacks the prion like domain between and including the amino acids at positions 274 to 414 of a wildtype TDP-43 polypeptide, optionally wherein the wildtype TDP-43 polypeptide comprises a sequence set forth as SEQ ID NO:1, SEQ ID NO:3, or SEQ ID NO:5. In some embodiments, a mutant TDP-43 polypeptide comprises F147L and F149L compared to a wildtype TDP-43 polypeptide, optionally wherein the wildtype TDP-43 polypeptide comprises a sequence set forth as SEQ ID NO:1, SEQ ID NO:3, or SEQ ID NO:5. In some embodiments, a mutant TDP-43 polypeptide comprises F194L and F229L compared to a wildtype TDP-43 polypeptide, optionally wherein the wildtype TDP-43 polypeptide comprises a sequence set forth as SEQ ID NO:1, SEQ ID NO:3, or SEQ ID NO:5 In some embodiments, a mutant TDP-43 polypeptide lacks the nuclear export signal between and including the amino acids at positions 239 and 250 compared to a wildtype TDP-43 polypeptide, optionally wherein the wildtype TDP-43 polypeptide comprises a sequence set forth as SEQ ID NO:1, SEQ ID NO:3, or SEQ ID NO:5.

The mutant TDP 43 polypeptide and nucleic acid molecules encoding same described herein are also provided. In some embodiments, the nucleic acid molecules encoding a mutant TDP-43 polypeptide as described herein further comprises from 5′ to 3′: a 5′ homology arm, the nucleic acid sequence encoding the mutant TDP-43 polypeptide, and a 3′ homology arm, wherein the nucleic acid undergoes homologous recombination in a rodent cell. In some embodiments, the 5′ and 3′ homology arms are homologous to rat sequences such that the nucleic acid undergoes homologous recombination at an endogenous rat TARDBP locus and the nucleic acid sequence encoding the mutant TDP-43 polypeptide replaces the endogenous TARDBP coding sequence. In some embodiments, the 5′ and 3′ homology arms are homologous to mouse sequences such that the nucleic acid undergoes homologous recombination at an endogenous mouse TARDBP locus and the nucleic acid sequence encoding the mutant TDP-43 polypeptide replaces the endogenous TARDBP coding sequence.

Also described herein are methods for making the non-human animals and non-human animal cells described herein. In some embodiments, the method comprises modifying the genome of the non-human animal or non-human animal cell to comprise a mutated TARDBP gene that encodes the mutant TDP 43 polypeptide, wherein the mutant TDP-43 polypeptide lacks a functional structural domain compared to a wildtype TDP-43, optionally wherein the wildtype TDP-43 polypeptide comprises a sequence set forth as SEQ ID NO:1, SEQ ID NO:3, or SEQ ID NO:5. In some embodiments, modifying comprises replacing an endogenous TARDBP gene with a mutated TARDBP gene that encodes a mutant TDP-43 polypeptide as described herein. In some embodiments, modifying further comprises replacing an endogenous TARDBP gene with a TARDBP gene comprising a knockout mutation, e.g., a conditional knockout mutation. In some embodiments, the methods further comprise culturing the cell in conditions that eliminates expression of the TARDBP gene comprising a knockout mutation.

Also described herein are methods of using the non-human animals, non-human animal cells, non-human animal tissues, and compositions. In some embodiments, the non-human animals, non-human animal cells, non-human animal tissues, and compositions are used in methods, e.g., methods of identifying a therapeutic candidate for the treatment of a disease and/or evaluating the biological function of a TDP-43 structural domain. In some embodiments of identifying a therapeutic candidate, the method comprising (a) contacting the non-human animal, non-human animal cell, non-human animal, or a composition comprising a non-human animal cell or tissue (e.g., an in vitro culture) as described herein with the candidate agent, (b) evaluating the phenotype and/or TDP-43 biological activity of the non-human animal, non-human cell or tissue, and (c) identifying the candidate agent that restores to the non-human animal, non-human cell or tissue a phenotype and/or TDP-43 biological activity comparable to that of a control cell or tissue that expresses a wildtype TDP-43 polypeptide.

In some embodiments of evaluating the biological function of a TDP-4, the methods comprise (a) modifying an embryonic stem (ES) cell to comprise a mutated TARDBP gene that encodes a mutant TDP 43 polypeptide that lacks a functional structural domain selected from the group consisting of the nuclear localization signal (NLS), the first RNA recognition motif (RRM1), the first RNA recognition motif (RRM2), the putative nuclear export signal (E), the prion like domain (PLD), and a combination thereof, (b) optionally differentiating the modified ES cell in vitro and/or obtaining a genetically modified non human animal from the modified ES cell, and (c) evaluating the phenotype and/or TDP-43 biological activity of the genetically modified ES cell, primitive ectoderm derived therefrom, motor neurons derived therefrom, or a non-human animal derived therefrom. In some embodiments, the method of claim 39 or claim 40, wherein the phenotype is evaluated by cell culture, fluorescence in situ hybridization, Western Blot analysis, or a combination thereof. In some embodiments, evaluating the phenotype comprises measuring the viability the genetically modified ES cell, primitive ectoderm derived therefrom, motor neurons derived therefrom, or a non-human animal derived therefrom. In some embodiments, evaluating the phenotype comprises determining the cellular location of the mutant TDP-43 polypeptide. In some embodiments, evaluating the biological activity of the mutant TDP-43 polypeptide comprises measuring the splice products of genes comprising cryptic exons regulated by TDP-43. In some embodiments, the gene comprising cryptic exons regulated by TDP-43 comprises Crem, Fyxd2, Clf1. In some embodiments, the biological activity of the mutant TDP-43 polypeptide comprises measuring the levels of an alternatively spliced TDP-43.

Also described herein are oligonucleotides (e.g., antisense oligonucleotides, siRNA, CRISPR/Cas system, etc.) that may be useful as candidate agents in treating a TDP-43 proteinopathy. In some embodiments, the antisense oligonucleotides comprise a gapmer motif targeting a TDP-43 mRNA sequence between the alternative 5′ and 3′ splice sites. In some embodiments, an antisense oligonucleotide comprises a gapmer motif targeting a TDP-43 mRNA sequence between alternative 5′ and 3′ splice sites, wherein the alternative 5′ splice site correlates to a TARDBP genomic position selected from the group consisting of (a) chromosome 4:148,618,647; (b) chromosome 4:148,618,665; and (c) chromosome 4:148,618,674, and wherein the alternative 3′ splice site correlates to a TARDBP genomic position of chromosome 4: 148,617,705. In some siRNA embodiments, the siRNA comprises a sequence targeting a TDP-43 mRNA sequence between the alternative 5′ and 3′ splice sites. In some embodiments, an siRNA comprising a sequence targets a TDP-43 mRNA sequence between alternative 5′ and 3′ splice sites, wherein the alternative 5′ splice site correlates to a TARDBP genomic position selected from the group consisting of (a) chromosome 4:148,618,647; (b) chromosome 4:148,618,665; and (c) chromosome 4:148,618,674, and wherein the alternative 3′ splice site correlates to a TARDBP genomic position of chromosome 4: 148,617,705. In some CRISPR/Cas system embodiments, the system comprises a Cas9 protein and at least one gRNA, wherein the gRNA recognizes a sequence at or near the 5′ alternative splice site and/or at or near the 3′ alternative splice site of a TDP-43 mRNA. In some embodiments, a CRISPR/Cas system comprises a Cas9 protein and at least one gRNA, wherein the gRNA recognizes a sequence at or near a TARDBP genomic position selected from the group consisting of (a) chromosome 4:148,618,647; (b) chromosome 4:148,618,665; (c) chromosome 4:148,618,674, (d) chromosome 4: 148,617,705 and a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides an illustration (not to scale) of TDP-43, the relative position for the nuclear localization signal (NLS; amino acids 82-98), the relative positions for the two RNA recognition motifs (RRM1; amino acids 106-176, and RRM2; amino acids 191-262), the relative position for a putative nuclear export signal (E; amino acids 239-248), the relative position for a prion like domain (PLD; amino acids 274-414), ALS-associated amino acid substitution mutations, and ALS-associated C terminal fragments. Asterisks highlight mutations associated with FTD symptoms with or without ALS. A90V, S92L, N267S, G287S, G294V, G368S, S375G, A382T, I383V, N390S, and N390D mutations have also been observed in healthy individuals.

FIG. 2A provides an illustration (not to scale) of the mouse TARDBP genomic structure, which depicts exons 1-6 (rectangles), untranslated regions (unfilled rectangles), and translated regions (filled rectangles) starting with the ATG start codon. FIG. 2B provides an amino acid sequence alignment of mouse (m) TDP-43 and human (h) TDP-43 polypeptides, the amino acid positions of the polypeptides, and a consensus sequence underneath the mTDP-43 and hTDP-43 sequences. Generally, boxed regions within the alignment show the nuclear localization signal (NLS: amino acids 82-98), RNA recognition motif 1 (RRM1: amino acids 106-176), RNA recognition motif 2 (RRM2: amino acids 191-262), a putative nuclear export signal (E: amino acids 239-248), and the glycine rich prion-like domain (PLD: amino acids 274-414). Amino acid mismatches between mouse TDP-43 and human TDP-43 are also boxed and depicted by a dash in the consensus sequence. Exon junctions are also depicted as vertical lines denoting the exons (EX) joined at the denoted junction. The vertical line between amino acids 286 and 287 provides an alternative 5′-splice site (see FIG. 11A).

FIG. 3A provides illustrations (not-to-scale) of two exemplary TARDBP null alleles: (1) a conditional knockout allele comprising exon 3 flanked by loxP site-specific recombination recognition sites (triangles), hereinafter referred to as “-” after removal of exon 3 upon cre-mediated recombination and (2) a TARDBP null allele comprising a deletion of the entire TARDBP coding sequence hereinafter referred to as “ΔCDS”. Depicted are exons 1-6 (rectangles), untranslated regions (unfilled rectangles), translated regions (filled rectangles), and relative locations of the start ATG and stop TGA codons. FIG. 3B provides illustrative depictions (not-to-scale) of non-limiting mutant TDP-43 polypeptides encoded by various forms of mutated TARDBP genes. Specifically, throughout these Examples and associated Figures:

“WT” refers to a wildtype TARDBP gene,
“loxP-Ex3loxP” refers to a mutated TARDBP gene comprising a floxed exon 3
“-” refers to a mutated TARDBP gene lacking a nucleotide sequence comprising the sequence of exon 3 of a wildtype TARDBP gene upon cre-mediated recombination of loxP-Ex3loxP,
“ΔCDS” refers to a mutated TARDBP gene lacking the entire coding sequence of TARDBP,
“ΔNLS” refers to a mutated TARDBP gene that encodes a mutant TDP-43 polypeptide comprising the following point mutations: K82A K83A, R84A, K95A, K97A, and K98A,
“ΔRRM1” refers to a mutated TARDBP gene that encodes a mutant TDP-43 polypeptide comprising the following point mutations: F147L and F149L,
“ΔRRM2” refers to a mutated TARDBP gene that encodes a mutant TDP-43 polypeptide comprising the following point mutations: F194L and F229L,
“ΔE” refers to a mutated TARDBP gene that encodes a mutant TDP-43 polypeptide lacking amino acids 239 to 250 of a wildtype TDP-43 polypeptide, and
“ΔPLD” refers to a mutated TARDBP gene that encodes a mutant TDP-43 polypeptide lacking amino acids 274 to 414 of a wildtype TDP-43 polypeptide.
For the ΔE and ΔPLD mutant TDP-43 polypeptides, diagonal lines represent regions that are deleted.

FIG. 4 illustrates a protocol used to differentiate embryonic stem (ES) cells into motor neurons. Also shown is the ability of ES cells comprising a mutated TARDBP gene as depicted to remain viable, reach the primitive ectoderm (PE) stage, and/or reach the motor neuron (MN) stage, after Cre-mediated deletion of exon 3 (-) at the ES cell stage.

FIG. 5 illustrates the protocol used to evaluate the viability of embryonic stem cell-derived motor neurons (ESMNs). Also shown is the result on viability of the ESMNs comprising a mutated TARDBP gene as indicated after activation of the conditional knockout allele (-).

FIG. 6A provides not-to-scale depictions of the regions of TDP-43 recognized by an anti-TDP-43 antibody that recognizes the N-terminus of TDP-43 (α-TDP-43 N-term) or an anti-TDP-43 antibody that recognizes the C-terminus of TDP-43 (α-TDP-43 C-term). FIG. 6B provides Western Blots of the cytoplasmic and nuclear fractions of cells stained with the antibody that recognizes the N-terminus of TDP-43 (α TDP-43 N-term) or the C-terminus of TDP-43 (α TDP-43 C-term) as depicted in FIG. 6A. Cre-mediated deletion of exon 3 (-) occurred at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). The cytoplasmic and nuclear fractions were isolated from TDP-43 WT/-modified ESMNs, ΔNLS/-modified ESMNs, ΔE/-modified ESMNs, ΔPLD/-modified ESMNs, or dying ΔRRM1/-modified cells. Graphs providing the ratio of cytoplasmic to nuclear TDP-43 of control TDP-43 WT/-ESMNs (•), ΔNLS/-modified ESMNs (▴), ΔRRM1/-modified cells (▾), or ΔPLD/-modified ESMNs (▪) are also provided.

FIG. 7 provides fluorescence in situ hybridization images at 40 magnification of modified embryonic stem cell derived motor neurons (ESMNs) comprising a mutated TARDBP gene as indicated. The images were captured after exon 3 of the mutated TARDBP gene was removed (-) at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). The cells were stained with an antibody that recognizes the C-terminus of TDP-43 (a TDP-43 C-term; top panels) or with an anti-MAP2 antibody and DAPI (bottom panels).

FIG. 8 provides fluorescence in situ hybridization images at 40 magnification of modified embryonic stem cell derived motor neurons (ESMNs) comprising a mutated TARDBP gene as indicated. The images were captured after exon 3 of the mutated TARDBP gene was removed (-) at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). The cells were stained with an antibody that recognizes the N-terminus of TDP-43 (a TDP-43 N-term; top panels) or with an anti-MAP2 antibody and DAPI (bottom panels).

FIG. 9A provides an anti-TDP-43 antibody stained Western Blot of the sarkosyl-soluble and sarkosyl-insoluble fractions of cells. Cre-mediated deletion of exon 3 (-) occurred at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). The sarkosyl-soluble and sarkosyl-insoluble fractions were isolated from TDP-43 WT/-modified ESMNs, ΔNLS/-modified ESMNs, ΔE/-modified ESMNs, ΔPLD/-modified ESMNs, or ΔRRM1/-modified cells. A graph providing the ratio of insoluble/soluble TDP-43 expressed by these ESMNs is also provided. FIG. 9B provides graphs showing TDP-43 mRNA (left panel; y-axis) or protein (right panel; y-axis) expression levels. Cre-mediated deletion of exon 3 (-) occurred at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). mRNA levels of ΔNLS/-modified ESMNs, ΔE/-modified ESMNs, ΔPLD/-modified ESMNs, or dying ΔRRM1/-modified cells are compared to a control (TDP-43WT/-modified ESMNs (WT/-)). FIG. 9C provides Western Blots stained with anti-TDP-43 or anti-GAPDH antibody of cell lysates. Cre-mediated deletion of exon 3 (-) occurred at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). Cell lysates were isolated from TDP-43 WT/-modified ESMNs, ΔNLS/-modified ESMNs, ΔE/-modified ESMNs, ΔPLD/-modified ESMNs, or dying ΔRRM1/-modified cells after cycloheximide (CHX+) treatment for up to 16 hours. A graph providing the % TDP-43 protein (y-axis) after cycloheximide treatment (x-axis; hrs) expressed by control TDP-43 WT/-modified ESMNs (•), ΔNLS/-modified ESMNs (▪), ΔRRM1/-modified cells (▴), or ΔPLD/-modified ESMNs (▾) is also provided.

FIG. 10 provides illustrations (not-to-scale) of normal and cryptic exon splicing that occurs in three genes thought to be regulated by TDP-43: Crem, Fyxd2, and Clf1, as well as graphs showing the levels of the normal spliced products (filled bars) and aberrant spliced products (patterned and unfilled bars. Cre-mediated deletion of exon 3 (-) occurred at the ES cell stage and cells were cultured with ES medium, ADFNK medium, ADFNK medium comprising retinoic acid and sonic hedgehog, and ESMN medium according to the protocol depicted in FIG. 4 to produce embryonic stem cell derived motor neurons (ESMNs). Levels of cryptic exon splicing of Crem, Fyxd2, and Clf1 by ΔNLS/-modified ESMNs, ΔE/-modified ESMNs, ΔPLD/-modified ESMNs, or ΔRRM1/-modified cells and a control (TDP-43 WT/-) are shown

FIG. 11A provides illustrations (not-to-scale) of normal and alternative splice events that occurs in the TDP-43 gene. FIG. 11B provides graphs showing the levels of the alternatively spliced TDP-43 mRNA. Cre-mediated deletion of exon 3 (-) occurred at the ES cell stage and cells were cultured with ADFNK medium, ADFNK medium comprising retinoic acid and sonic hedgehog, and ESMN medium according to the protocol depicted in FIG. 4 to produce embryonic stem cell derived motor neurons (ESMNs). The levels of the alternatively spliced TDP-43 mRNA by unmodified ES cells (WT/WT), ΔNLS/-modified ESMNs, ΔE/-modified ESMNs, ΔPLD/-modified ESMNs, or dying ΔRRM1/-modified cells are shown.

FIG. 12 provides a graph showing the survival time post fertilization of 8-cell embryos injected with TDP-43^{-/-} ES cells, TDP-43^{ΔNLS/-} modified ES cells, TDP-43^{ΔPLD/-} modified ES cells, TDP-43^{ΔNLS/WT} modified ES cells, TDP-43^{ΔPLD/WT} modified ES cells, TDP-43^{WT/-} modified ES cells, TDP-43^{loxP-Ex3-loxP/WT} modified ES cells, or wildtype TDP-43^{WT/WT} ES cells. E3.5 (embryonic day 3.5), E 10.5 (embryonic day 10.5), E 15.5 (embryonic day 15.5), P0 (postnatal day 0).

FIGS. 13A, 13B and 13C provide Western Blots of motor neurons isolated from spinal cord tissue isolated from 16 week old mice (n=2). The mice examined expressed from (i) an endogenous TARDBP locus: a mutated TARDBP gene comprising a floxed exon 3 (loxP-Ex3-loxP), a mutated TARDBP gene comprising knockout mutations in the NLS (ΔNLS), or a mutated TARDBP gene comprising a deletion of the prion like domain (ΔPLD), and (ii) at the other TARDBP locus on a homologous chromosome, a wildtype (WT) TARDBP gene. FIG. 13A shows the cytoplasmic and nuclear fractions of the motor neurons stained with the respective α-TDP-43 N-term or α-TDP-43 C-term antibody that recognizes the N-terminus of TDP-43 or the C-terminus of TDP-43 (see, e.g., FIG. 6A). Graphs providing the ratio of cytoplasmic to nuclear TDP-43 of spinal cord tissue isolated from the loxP-Ex3-loxP/WT mice (•), the ΔNLS/WT mice (▴), or the APLD/WT mice (▾) are also provided. FIG. 13B provides Western Blots of the cytoplasmic and nuclear fractions of spinal cord tissue isolated with 16 week old mice and stained with an antibody that recognizes phosphorylated TDP-43. FIG. 13C provides Western Blots of the sarkosyl-soluble and sarkosyl-insoluble fractions of cells stained with the respective α-TDP-43 N-term (see, e.g., FIG. 6A) or α-TDP-43 C-term antibodies (see, e.g., FIG. 6A) that recognizes the N-terminus of TDP-43 or the C-terminus of TDP-43.

FIG. 14 provides fluorescence in situ hybridization images at 40× magnification of motor neurons isolated from spinal cord tissue isolated from 16 week old mice. The mice examined expressed from (i) an endogenous TARDBP locus: a mutated TARDBP gene comprising a foxed exon 3 (loxP-Ex3-loxP), a mutated TARDBP gene comprising knockout mutations in the NLS (ΔNLS), or a mutated TARDBP gene comprising a deletion of the prion like domain (ΔPLD), and (ii) at the other TARDBP locus on a homologous chromosome, a wildtype (WT) TARDBP gene. The cells were stained with an antibody that recognizes the N-terminus of TDP-43 (α TDP-43 M-term; top panels) or with anti-chAT antibody and anti-NeuN antibodies (bottom panels). Also shown is a graph providing the percentage of motor neurons exhibiting cytoplasmic aggregates in animals expressing only wildtype TDP-43 (•), the mutant ΔNLS TDP-43 polypeptide and wildtype TDP-43 polypeptide (▪), both the mutant ΔNLS TDP-43 polypeptide and wildtype TDP-43 polypeptide (▪), or both the mutant ΔPLD TDP-43 polypeptide and wildtype TDP-43 polypeptide (▴).

FIG. 15A provides fluorescence in situ hybridization images at 10× or 40× magnification of tibialis anterior muscle tissue or intercostal muscle tissue isolated from 16 week old mice. The tissues were stained with an antibody that recognizes synaptophysin, bungarotoxin, and/or DAPI. Arrows indicate denervated muscular junctions, and asterisks indicate partially innervated neuromuscular junction. FIG. 15B are graphs providing the percent innervated neuromuscular junctions (NMJs; y-axis) in tibialis anterior (TA) muscle tissue or intercostal muscle isolated from the loxP-Ex3-loxP/WT mice (•), the ΔNLS/WT mice (▴), or the ΔPLD/WT mice (▾).

### DETAILED DESCRIPTION

Overview

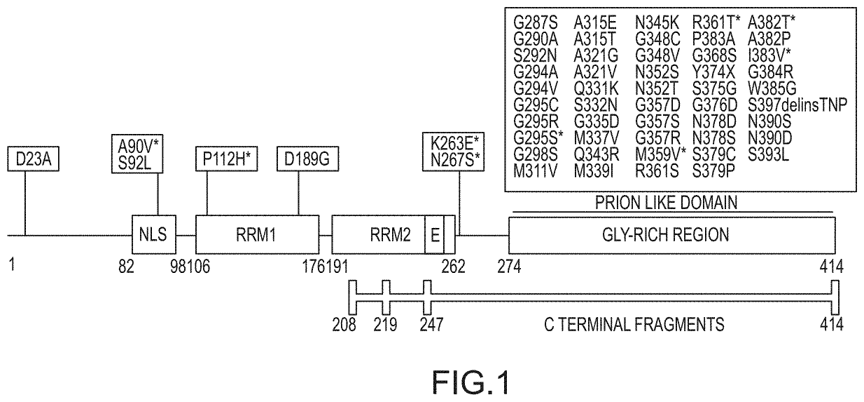

TDP-43 is a predominantly nuclear RNA/DNA-binding protein that functions in RNA processing and metabolism, including RNA transcription, splicing, transport, and stability. The RNA-binding properties of TDP-43 appear essential for its autoregulatory activity, mediated through binding to 3′ UTR sequences in its own mRNA. Ayala et al. (2011) EMBO J. 30:277-88. Following cell stress, TDP-43 localizes to cytoplasmic stress granules and may play a role in stress granule formation. TDP-43 mislocalizes from its normal location in the nucleus to the cytoplasm, where it aggregates. Aggregated TDP-43 is ubiquinated, hyperphosphorylated, and truncated. Additionally, TDP-43 aggregation in the cytoplasm is a component of nearly all cases of ALS. Becker et al. (2017) Nature 544:367-371. Ninety-seven percent of ALS cases show a post-mortem pathology of cytoplasmic TDP-43 aggregates. The same pathology is seen in approximately 45% of sporadic Frontotemporal Lobar Degeneration (FTLDU). TDP-43 was first identified as the major pathologic protein of ubiquitin-positive, tau-negative inclusions of FTLDU, FTLD with motor neuron disease (FTDMND), and ALS/MND (ALS10), which disorders are now considered to represent different clinical manifestations of TDP-43 proteinopathy. Gitcho et al. (2009) Acta Neuropath 118:633-645. TARDBPB mutations occur in about 3% of patients with familial ALS and in about 1.5% of patients with sporadic disease. Lattante et al. (2013) Hum. Mutat. 34:812-26. Various mutations in the TARDBP gene have been associated with ALS in less than 1% of the cases. See FIG. 1. As shown in FIG. 1, the majority mutations in the TARDBP gene associated with ALS is found in the prion like domain (PLD). Therefore, understanding all the functions played by TDP-43 would likely elucidate its role in neuropathologies such as ALS, FLTDU, and FLTD, etc.

It is clear that TDP-43 is essential for cellular and organismal life. Depletion of TDP-43 results in embryonic lethality. Accordingly, initial models relied on the overexpression of TDP-43 or mutant forms thereof, or deletion of TDP-43. Various models evaluating the role of TDP-43 in ALS pathologies have been created. Reviewed in Tsao et al (2012) Brain Res 1462:26-39.

For example, transgenic mice overexpressing a TDP-43 A315T mutant developed progressive abnormalities at about 3 to 4 months of age and died at about 5 months of age. Wegorzewska et al (2009) Proc Natl Acad Sci USA 106:18809-814. Although the abnormalities were correlated with the presence of TDP-43 C-terminal fragments in the brain and spinal cord of these mutant mice, cytoplasmic TDP-43 aggregates were not detected. These observations led Wegorzewska et al. to suggest that neuronal vulnerability to TDP-43 associated neurodegeneration is related to altered DNA/RNA-binding protein function rather than toxic aggregation. Wegorzewska et al. (2009), supra. In contrast, in two independent studies involving the overexpression of TDP-43, transgenic mice exhibited neurodegenerative attributes including progressive motor dysfunction that was correlated with cytoplasmic aggregation. Tsai et al. (2010) J. Exp. Med. 207:1661-1673 and Wils et al (2010) Proc Natl Acad Sci USA 107:3858-63).

In loss-of function studies, ubiquitous deletion of TDP-43 using a conditional knockout mutation led to mice exhibiting a metabolic phenotype and premature death. Chiang et al. (2010) Proc Natl Acad Sci USA 107:16320-324. Depletion of TDP-43 in mouse embryonic stem cells resulted in the splicing of cryptic exons of certain genes into mRNA, disrupting translation of the mRNA and promoting nonsense-mediated mRNA decay. Ling et al. (2015) Science 349:650-655. Since postmortem brain tissue from patients with ALS/FTD show impaired repression of cryptic exon splicing, this study suggests that TDP-43 normally acts to repress the splicing of cryptic exons and maintain intron integrity, and that TDP-43 splicing defects could contribute to TDP-43-proteinopathy in certain neurodegenerative disease. Ling et al. (2015), supra. Since point mutations in the N-terminus (e.g., the NLS) of TDP-43 result in destabilization of TDP-43 oligomerization in the nucleus and loss of cryptic splicing regulation, it is hypothesized that head-to-tail oligomerization of TDP-43 driven by the N-terminus acts to separate the aggregation prone C-terminus domain (e.g., the PLD), and thus, prevent the formation of pathologic aggregates. Afroz et al. (2017) Nature Communications 8:45.

In ALS, one of the first pathological features to manifest is that the axon retracts from the neuromuscular junction causing the muscle to denervate. This denervation continues to progress resulting in the loss of the motor neuron cell body and muscle atrophy. Denervation may be observed by the loss of presynaptic markers of axon innervation: VAChT, Synaptic vesicle protein 2 (SV2), synaptophysin, and neurofilament. The motor endplate remains but will eventually fragment and disappear. Recently, dose-dependent denervation was exhibited in mice homozygous for a knockin TARDBP gene comprising disease-associated mutations. Ebstein (2019) Cell Reports 26:364-373.

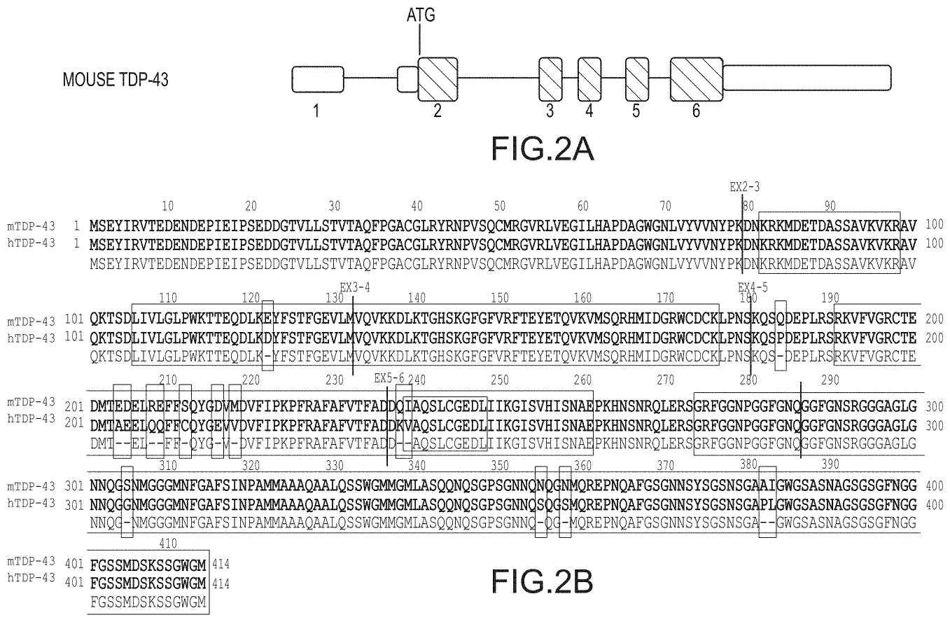

Despite embryonic lethality of TDP-43 depletion, we show here that embryonic stem (ES) cells expressing a TDP-43 mutant lacking a functional structural domain remain viable and may be differentiated into motor neurons (ESMNs). See, FIGS. 4-5. These observations are unique in that the ES or ESMNs as described herein express a mutant TDP-43 polypeptide that:
(1) lacks a functional structural domain, e.g., lacks a functional NLS, lacks a functional RRM1, lacks a functional RRM2, lacks a functional E, or lacks a functional PLD, and
(2) is expressed at normal levels from an endogenous transcriptional promoter and pre-mRNA splicing signals. See, e.g., FIG. 2 and FIG. 9.
   Using the ES and ESMNs described herein, it is shown that RRM1 is required for viability of ES cells and motor neurons derived therefrom. See, FIGS. 4-5. Moreover, expression of mutant TDP-43 polypeptides (1) lacking a functional NLS or a functional PLD and (2) at normal levels from the endogenous locus reproduces two hallmarks of ALS disease in ESMNs:

(i) redistribution of TDP-43 from the nucleus to the cytoplasm, and

(ii) accumulation in cytoplasmic inclusions. See, FIGS. 6-8.

It is surprising that ΔPLD mutants, i.e., TDP-43 polypeptides comprising a functional NLS but lacking a PLD, aggregate in the cytoplasm. See, e.g., Afroz et al. (2017), supra. Notably, the punctate inclusions formed by ΔPLD mutants appear to be less abundant and qualitatively different than inclusions formed by ΔNLS mutants, i.e., TDP-43 polypeptides lacking a functional NLS and comprising a PLD. Furthermore, the ALS-like phenotype of ESMNs expressing a ΔPLD or ΔNLS is correlated with both a decrease in repression of cryptic exon splicing of genes, for which splice events are usually regulated by wildtype TDP-43. FIG. 9. Also shown is a correlation in ESMNs between expression of a ΔPLD or ΔNLS mutated TARDBP gene and a decrease in an alternative splice event involving a 3′untranslated region intron that results in an alternative spliced TDP-43 mRNA lacking sequences encoding the PLD domain, or portion thereof and the stop codon. FIG. 10; see also Avendano-Vazquez et al. (2012) Genes & Dev. 26:1679-84; Ayala Y M, et al. (2011) EMBO J 30: 277-288. This latter observation suggests that depleting only wildtype or ALS-associated sequences resulting from normal splice events may be potentially therapeutic for the treatment of ALS associated with PLD mutations.

Mice expressing a wildtype TARDBP gene and a ΔPLD or ΔNLS mutated TARDBP gene from endogenous loci also exhibited hallmarks of TDP-43 proteinopathies. Increased TDP-43 mislocalization from the nucleus to the cytoplasm, phosphorylation of cytoplasmic TDP-43, and cytoplasmic aggregation of TDP-43 was observed in spinal cord motor neurons of animals expressing mutant ΔPLD or ΔNLS TDP-43 polypeptides compared to animals expressing only wildtype protein (FIGS. 13A-13B, and 14). TDP-43 mutants lacking a functional NLS, but not TDP-43 mutants lacking a PLD, were insoluble (FIG. 13C). Moreover, denervation of muscles comprised mostly of fast twitch fibers, but not of muscles comprised mostly of slow twitch fibers, was also observed in these mice expressing mutant ΔPLD or ΔNLS TDP-43 proteins (FIGS. 15A-B).

The discoveries provided herein provide not only a method of evaluating TDP-43 mutations in viable embryonic stem (ES) cells, and tissues and non-human animals derived therefrom (e.g., primitive ectoderm, motor neurons derived therefrom (ESMNs), but also ES cells, ESMN cells, and non-human animals that express mutant TDP-43 polypeptides lacking a functional structural domain. ES, ESMN cells, an non-human animals (e.g., rodents, e.g., rats and mice) expressing a mutant TDP-43 polypeptide lacking a functional structural domain may also respectively be used as in vitro or in vivo models of TDP-43 proteinopathy, e.g., in methods of identifying a therapeutic candidate for same.

TARDBP Genes and TDP-43 Polypeptides

A TARDBP gene encodes a TDP-43polypeptide, also referred to as TAR DNA-binding protein, TARDBP, 43-KD, and TDP43, and TDP-43. The nucleic acid sequence of wildtype TARDBP genes and the wildtype TDP-43 polypeptides encoded therefrom of different species are well known in the art. For example, the respective nucleic acid and amino acid sequences of wildtype TARDBP genes and wildtype TDP-43 polypeptides and may be found in the U.S. National Library of Medicine National Center for Biotechnology Information (NCBI) gene database. See, e.g., the website at www.ncbi.nlm.nih.gove/gene/?term=TARDBP. In some embodiments, a wildtype mouse TARDBP gene comprises a nucleotide sequence that encodes a wildtype mouse TDP-43 polypeptide comprising an amino acid sequence set forth as GenBank accession number NP_663531 (SEQ ID NO:1), or a variant thereof that differs from same due to a conservative amino acid substitution. In some embodiments, a wildtype mouse TARDBP gene comprises a nucleic acid sequence set forth as GenBank accession number NM_145556.4 (SEQ ID NO:2), or a variant thereof that differs from same due to degeneracy of the genetic code and/or a conservative codon substitution. In some embodiments, a wildtype rat TARDBP gene comprises a nucleotide sequence that encodes a wildtype rat TDP-43 polypeptide comprising an amino acid sequence set forth as GenBank accession number NP_001011979 (SEQ ID NO:3), or a variant thereof that differs from same due to a conservative amino acid substitution. In some embodiments, a wildtype rat TARDBP gene comprises a nucleic acid sequence set forth as GenBank accession number NM_001011979.2 (SEQ ID NO:4), or a variant thereof that differs from same due to degeneracy of the genetic code and/or a conservative codon substitution. In some embodiments, a wildtype human TARDBP gene encodes a TDP-43 polypeptide comprising an amino acid set forth as GenBank accession number NP_031401.1 (SEQ ID NO:5), or a variant thereof that differs from same due to a conservative amino acid substitution. In some embodiments, a wildtype human TARDBP gene comprises a nucleic acid sequence set forth as GenBank accession number NM_007375.3 (SEQ ID NO:6), or a variant thereof that differs from same due to degeneracy of the genetic code and/or a conservative codon substitution.

Described herein is a mutated TARDBP gene. A mutated TARDBP gene may comprise a knockout mutation. A mutated TARDBP gene may encode a mutant TDP-43 polypeptide, wherein the mutant TDP-43 polypeptide lacks a functional structural domain. For example, a mutated TARDBP gene may comprise a nucleotide sequence encoding a TDP-43 structural domain comprising a point mutation, an insertion within, and/or deletion of a portion or all of the structural domain, wherein the point mutation, insertion, and/or deletion results in a loss-of-function of the structural domain, and wherein the mutated TARDBP gene still encodes a TDP-43 polypeptide, albeit a mutant TDP-43 polypeptide lacking a functional structural domain due to the mutation. A polypeptide may be referred to as a mutant TDP-43 polypeptide wherein it comprises at least one wildtype TDP-43 structural domain or variant thereof and/or wherein it is specifically bound by an anti-TDP-43 antibody or antigen binding portion thereof. Similarly, a mutated TARDBP gene may be so classified wherein the mutated TARDBP gene encodes a mutant TDP-43 polypeptide, e.g., a polypeptide that comprises at least one wildtype TDP-43 structural domain or variant thereof and/or may be specifically bound by an anti-TDP-43 antibody or antigen binding portion thereof.

The structural domains of TDP-43 have been identified as a nuclear localization signal (NLS), two RNA recognition motifs (RRM1 and RRM2), a putative nuclear export signal (E), and a glycine rich prion like domain (PLD). See FIGS. 1 and 2. A wildtype TDP-43 polypeptide comprises a TDP-43 NLS at amino acids 82-99, a TDP-43 RRM1 at amino acids 106-176, a TDP-43 RRM2 at amino acids 191-262, a TDP-43 E at amino acids 239-248, and a TDP-43 PLD at amino acids 274-414.

Classical NLS sequences comprise stretches of basic amino acids, primarily lysine (K) and arginine (R) residues, and bipartite NLS comprise two clusters of these basic amino acids separated by a linker region comprising about 10-13 amino acids. An amino acid substitution and/or deletion of a basic amino acid sequence of a classical NLS may abolish function of the classical NLS. McLane and Corbett (2009) IUBMB Life 61:697-706. A TDP-43 NLS comprises lysine and arginine residues at positions 82, 83, 84, 95, 97, and 98. A wildtype TDP-43 polypeptide modified to comprise an amino acid substitution and/or deletion at positions 82, 83, 84, 95, 97, and/or 98 may lack a functional NLS. A mutant TDP-43 polypeptide lacking a functional NLS may comprise an amino acid sequence set forth in SEQ ID NO:1 modified to comprise an amino acid substitution and/or deletion at positions 82, 83, 84, 95, 97, and/or 98. A mutant TDP-43 polypeptide lacking a functional NLS may comprise an amino acid sequence set forth in SEQ ID NO:3 modified to comprise an amino acid substitution and/or deletion at positions 82, 83, 84, 95, 97, and/or 98. A mutant TDP-43 polypeptide lacking a functional NLS may comprise an amino acid sequence set forth in SEQ ID NO:5 modified to comprise an amino acid substitution and/or deletion at positions 82, 83, 84, 95, 97, and/or 98. Accordingly, a mutated TARDBP gene that encodes a mutant TDP-43 protein lacking a functional TDP-43 NLS may comprise a sequence encoding a TDP-43 polypeptide comprising a sequence set forth as SEQ ID NO:1, SEQ ID NO:3, or SEQ ID NO:5 modified to comprise (i) an amino acid substitution at a position selected from the group consisting of 82, 83, 84, 95, 97, and/or 98, and a combination thereof, and/or (ii) a deletion of any amino acids at and between portions 82 and 98. A mutated TARDBP gene that encodes a mutant TDP-43 protein lacking a functional TDP-43 NLS may comprise a nucleotide sequence encoding an amino acid sequence set forth as SEQ ID NO:1, SEQ ID NO:3 or SEQ ID NO:5 modified to comprise an amino acid substitution selected from the group consisting of K82A K83A, R84A, K95A, K97A, K98A or a combination thereof. A mutated TARDBP gene that encodes a mutant TDP-43 protein lacking a functional TDP-43 NLS may comprise a nucleotide sequence encoding an amino acid sequence set forth as SEQ ID NO:1, SEQ ID NO:3 or SEQ ID NO:5 modified to comprise following amino acid substitutions: K82A K83A, R84A, K95A, K97A, and K98A.

RNA binding by a typical RRM is usually achieved by contacts made between the surface of a four-stranded antiparallel β sheet of the typical RRM and a single stranded RNA. Melamed et al. (2013) RNA 19:1537-1551. Two highly conserved motifs, RNP1 (consensus K/R-G-F/Y-G/A-F/Y-V/I/L-X-F/Y, where X is any amino acid) and RNP2 (consensus I/V/L-F/Y-I/V/L-X-N-L, where X is any amino acid) in the central two β strands, are the primary mediators of RNA binding. Melamed et al. (2013), supra.

A TDP-43 RRM1, located at amino acid positions 106-176 of a wildtype TDP-43 polypeptide comprises an RNP2 consensus sequence (LIVLGL; SEQ ID NO:7) located at amino acid positions 106-111 and an RNP1 consensus sequence (KGFGFVRF; SEQ ID NO:8) located at amino acid positions 145-152. Previously, W113, T115, F147, F149, D169, R171, and N179 were identified as critical residues for nucleic acid binding. A wildtype TDP-43 polypeptide modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv), may lack a functional RRM1. A mutant TDP-43 polypeptide lacking a functional RRM1 may comprise a sequence set forth as SEQ ID NO:1 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv). A mutant TDP-43 polypeptide lacking a functional RRM1 may comprise a sequence set forth as SEQ ID NO:3 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv). A mutant TDP-43 polypeptide lacking a functional RRM1 may comprise a sequence set forth as SEQ ID NO:5 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152, or (v) any combination of (i)-(iv), Accordingly, a mutated TARDBP gene encoding a mutant TDP-43 polypeptide lacking a functional RRM1 may comprise a nucleotide sequence that encodes a TDP-43 polypeptide comprising an amino acid sequence set forth as SEQ ID NO:1, SEQ ID NO:3, or SEQ ID NO:5 modified to comprise (i) an amino acid substitution at a position selected from the group consisting of 113, 115, 147, 149, 169, 171, 179 and any combination thereof, (ii) a deletion or substitution of any amino acids at and between positions 106-176, (iii) a deletion or substitution of any amino acids at and between positions 106-111, (iv) a deletion or substitution of any amino acids at and between of 145-152 of a wildtype TDP-43 polypeptide, or (v) any combination of (i)-(iv). A mutated TARDBP gene encoding a mutant TDP-43 polypeptide lacking a functional RRM1 may comprise a nucleotide sequence that encodes a TDP-43 polypeptide comprising an amino acid sequence set forth as SEQ ID NO:1, SEQ ID NO:3, or SEQ ID NO:5 modified to comprise a F147L and/or F149L mutation. A mutated TARDBP gene encoding a mutant TDP-43 polypeptide lacking a functional RRM1 may comprise a nucleotide sequence that encodes a TDP-43 polypeptide comprising an amino acid sequence set forth as SEQ ID NO:1, SEQ ID NO:3, or SEQ ID NO:5 modified as to comprise the following amino acid substitutions: F147L and F149L.

A TDP-43 RRM2, located at amino acid positions 191-262 of a wildtype TDP-43 polypeptide comprises an RNP2 consensus sequence (VFVGRC; SEQ ID NO:9) located at amino acid positions 193-198 and an RNP1 consensus sequence (RAFAFVT; SEQ ID NO:10) located at amino acid positions 227-233. F194 and F229 may be considered critical residues for nucleic acid binding. A wildtype TDP-43 polypeptide modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(v), may lack a functional RRM2. A mutant TDP-43 polypeptide lacking a functional RRM2 may comprise a sequence set forth as SEQ ID NO:1 modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(iv). A mutant TDP-43 polypeptide lacking a functional RRM2 may comprise a sequence set forth as SEQ ID NO:3 modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(iv). A mutant TDP-43 polypeptide lacking a functional RRM2 may comprise a sequence set forth as SEQ ID NO:5 modified to comprise (i) an amino acid substitution at a position selected from the group consisting of 194 and/or 229, (ii) a deletion or substitution of any amino acids at and between positions 193-198, (iii) a deletion or substitution of any amino acids at and between positions 227-233, (iv) a deletion or substitution of any amino acids at and between of 191-262, or (v) any combination of (i)-(iv). Accordingly, a mutated TARDBP gene encoding a mutant TDP-43 polypeptide lacking a functional RRM2 may comprise a nucleotide sequence encoding a TDP-43 polypeptide comprising an amino acid sequence set forth as SEQ ID NO:1, SEQ ID NO:3, or SEQ ID NO:5 modified to comprise (i) an amino acid substitution at positions 194 and/or 229 of a wildtype TDP-43 polypeptide (ii) a deletion or substitution of any amino acids at and between positions 191-262, or (iii) both (i) and (ii). A mutated TARDBP gene encoding a mutant TDP-43 polypeptide lacking a functional RRM2 may comprise a nucleotide sequence encoding a TDP-43 polypeptide comprising an amino acid sequence set forth as SEQ ID NO:1, SEQ ID NO:3, or SEQ ID NO:5 modified to comprise a F194L and/or F229L mutation. A mutated TARDBP gene encoding a mutant TDP-43 polypeptide lacking a functional RRM2 may comprise a nucleotide sequence encoding a TDP-43 polypeptide comprising an amino acid sequence set forth as SEQ ID NO:1, SEQ ID NO:3, or SEQ ID NO:5 modified to comprise a F194L and a F229L mutation.

A nuclear export signal of a wildtype TDP-43 polypeptide may be located at amino acids 239-248. A mutant TDP-43 polypeptide lacking a functional nuclear export signal may comprise an amino acid sequence set forth as SEQ ID NO:1 modified to comprise a deletion of any amino acids at and between positions 236-251. A mutant TDP-43 polypeptide lacking a nuclear export signal may comprise an amino acid sequence set forth as SEQ ID NO:1 modified to comprise a deletion of at least amino acids 239-250. A mutant TDP-43 polypeptide lacking a nuclear export signal may comprise an amino acid sequence set forth as SEQ ID NO:3 modified to comprise a deletion of any amino acids at and between positions 236-251. A mutant TDP-43 polypeptide lacking a nuclear export signal may comprise an amino acid sequence set forth as SEQ ID NO:3 modified to comprise a deletion of at least amino acids 239-250. A mutant TDP-43 polypeptide lacking a nuclear export signal may comprise an amino acid sequence set forth as SEQ ID NO:5 modified to comprise a deletion of any amino acids at and between positions 236-251. A mutant TDP-43 polypeptide lacking a nuclear export signal may comprise an amino acid sequence set forth as SEQ ID NO:5 modified to comprise a deletion of at least amino acids 239-250. Accordingly, a mutated TARDBP gene encoding a mutant TDP-43 polypeptide lacking a functional nuclear export signal may comprise a nucleotide sequence encoding a TDP-43 polypeptide comprising an amino acid sequence set forth as SEQ ID NO:1, SEQ ID NO:3, or SEQ ID NO:5 modified to comprise a deletion of amino acids at and between 236-251, e.g., a deletion of amino acids at and between 239-250.

A prion like domain (PLD) of a wildtype TDP-43 polypeptide may be located at amino acids 274-414. A mutant TDP-43 polypeptide lacking a functional PLD may comprise an amino acid sequence set forth as SEQ ID NO:1 modified to comprise a deletion of at least one or all amino acids at and between positions 274-414. A mutant TDP-43 polypeptide lacking a functional PLD may comprise an amino acid sequence set forth as SEQ ID NO:3 modified to comprise a deletion of at least one or all amino acids at and between positions 274-414. A mutant TDP-43 polypeptide lacking a functional PLD may comprise an amino acid sequence set forth as SEQ ID NO:5 modified to comprise a deletion of at least one or all amino acids at and between positions 274-414. Accordingly, a mutated TARDBP gene that encodes a mutant TDP-43 polypeptide may comprise a nucleotide sequence encoding a TDP-43 polypeptide comprising an amino acid sequence set forth as SEQ ID NO:1, SEQ ID NO:3 or SEQ ID NO:5 modified to comprise a deletion of at least one or all amino acids at and between positions 274-414.

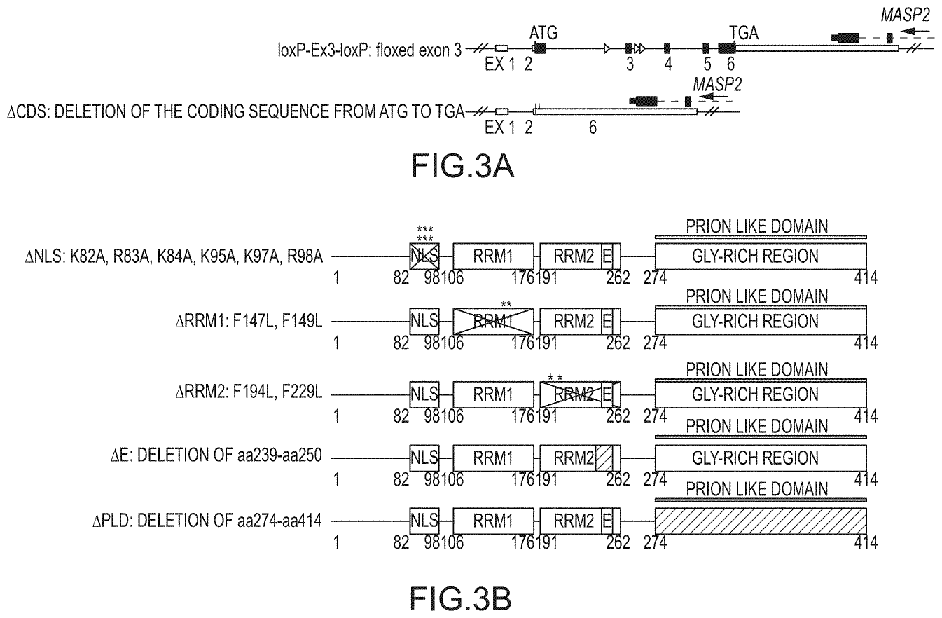

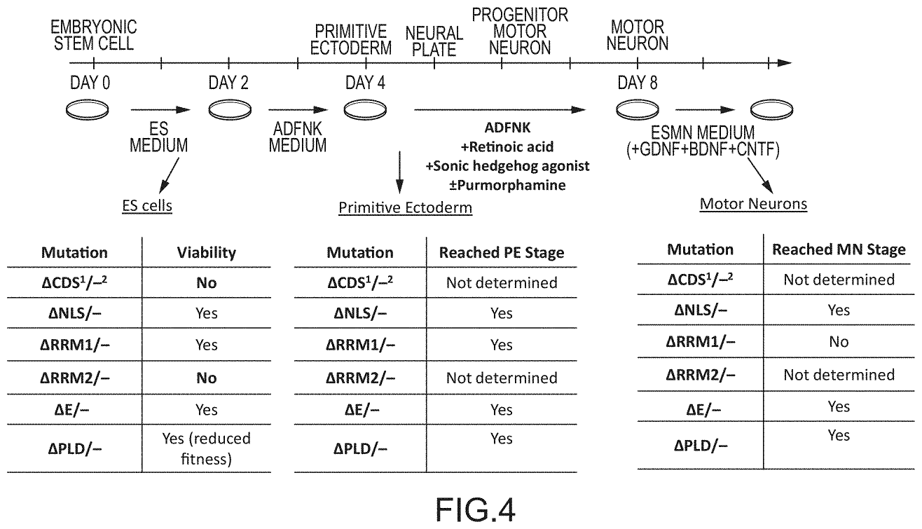

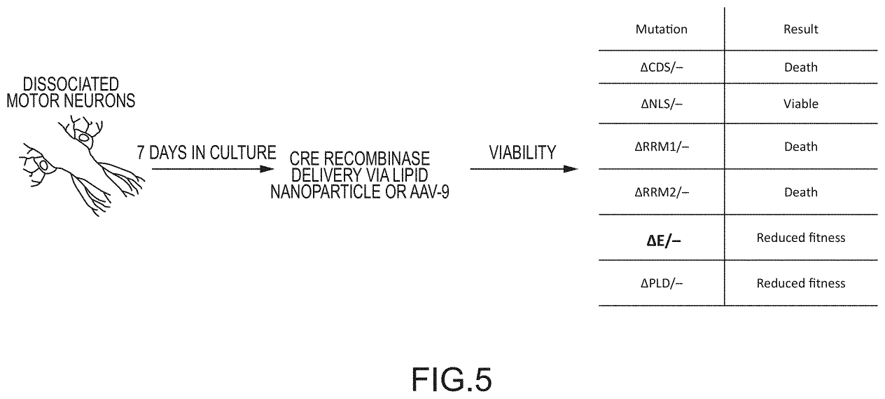

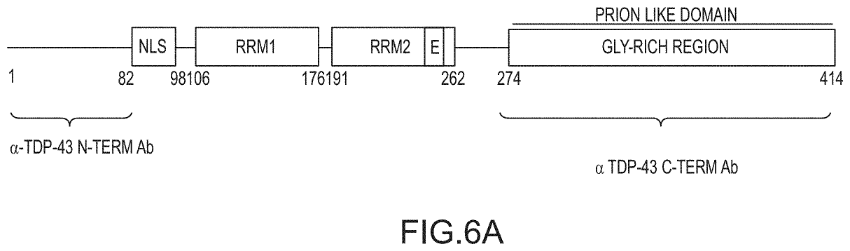

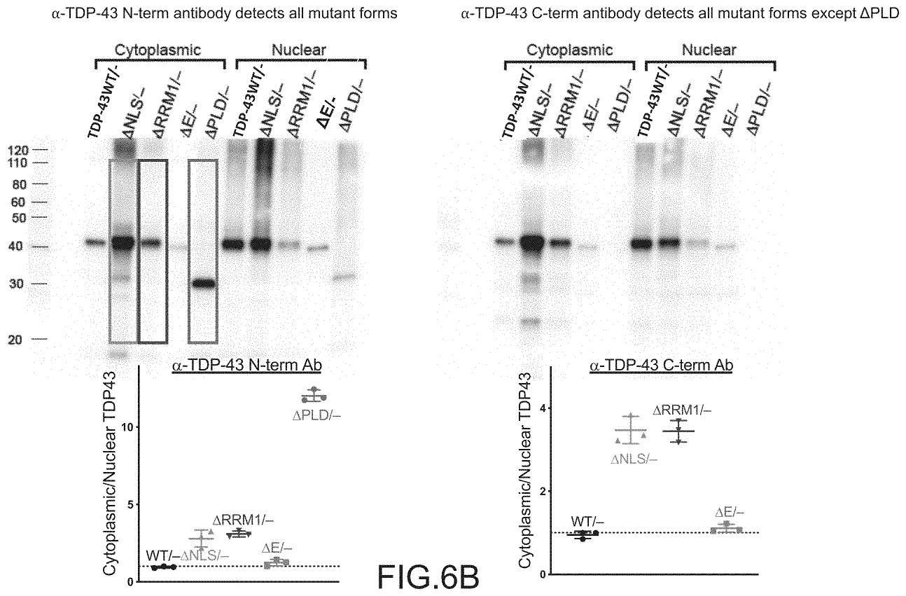

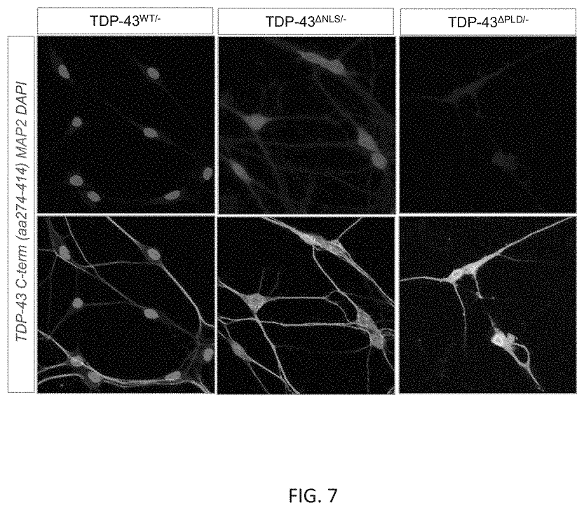

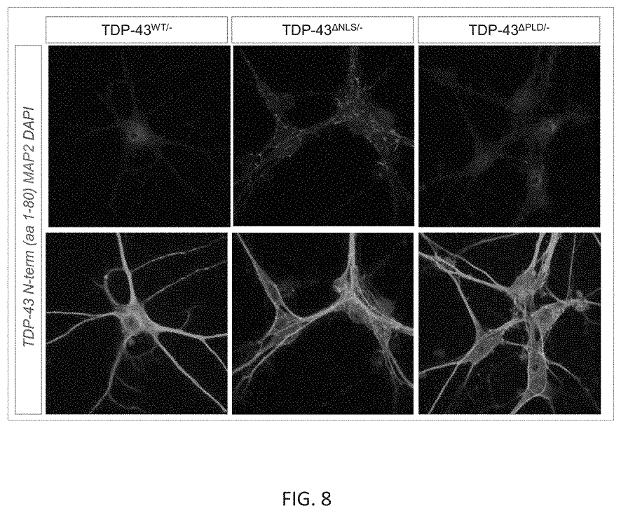

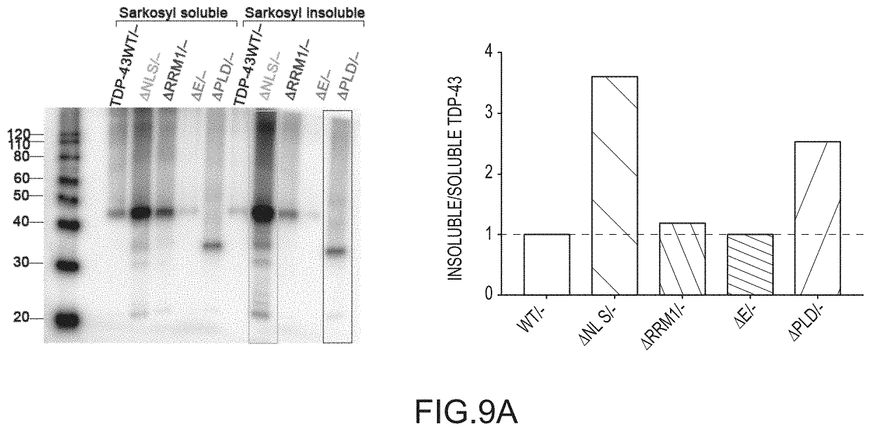

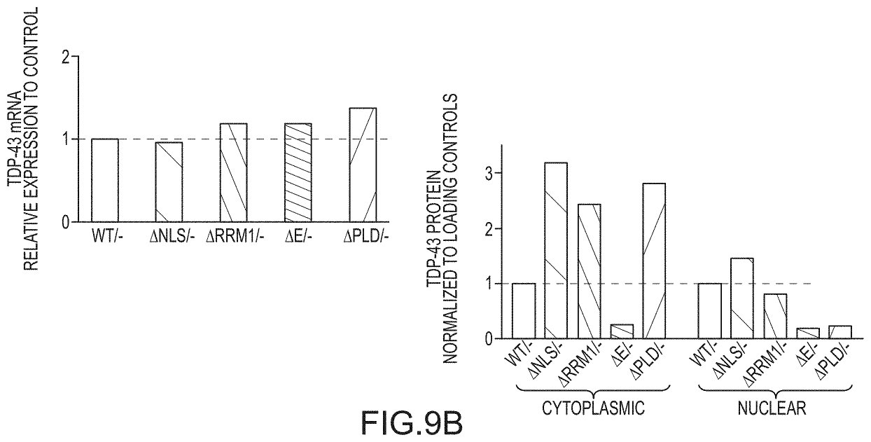

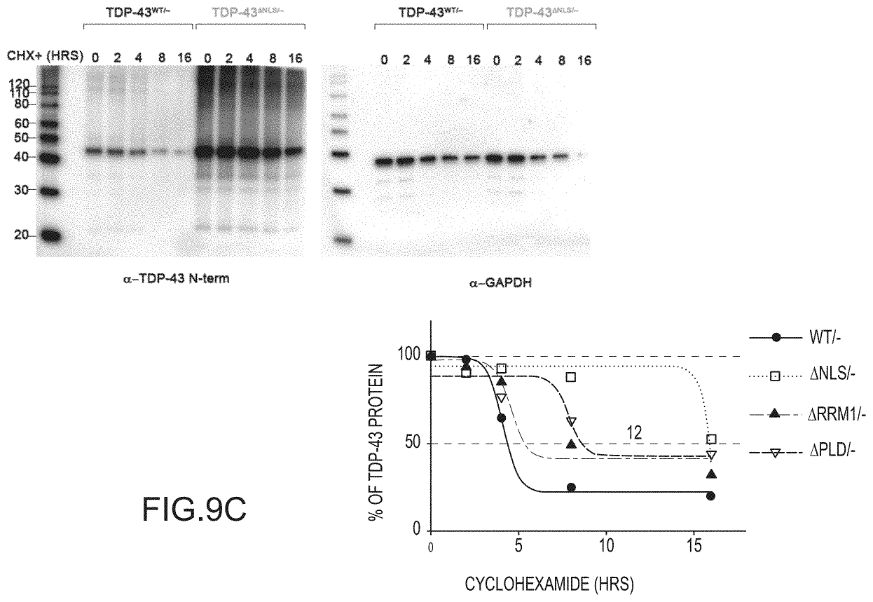

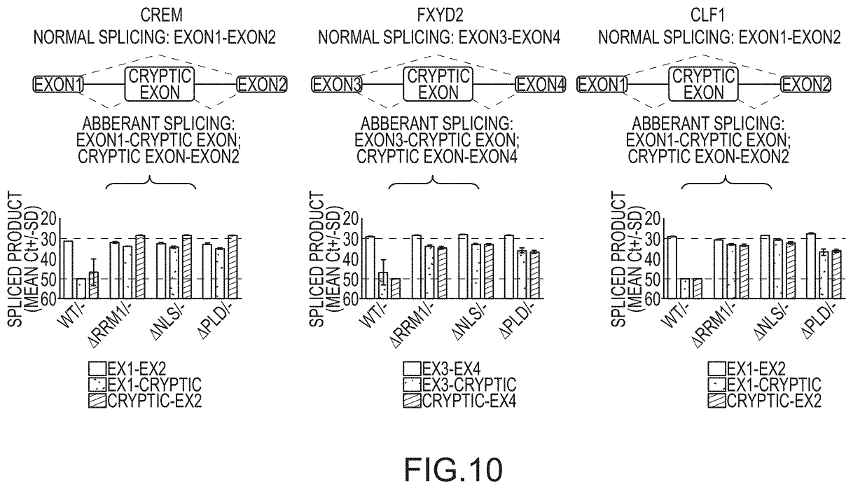

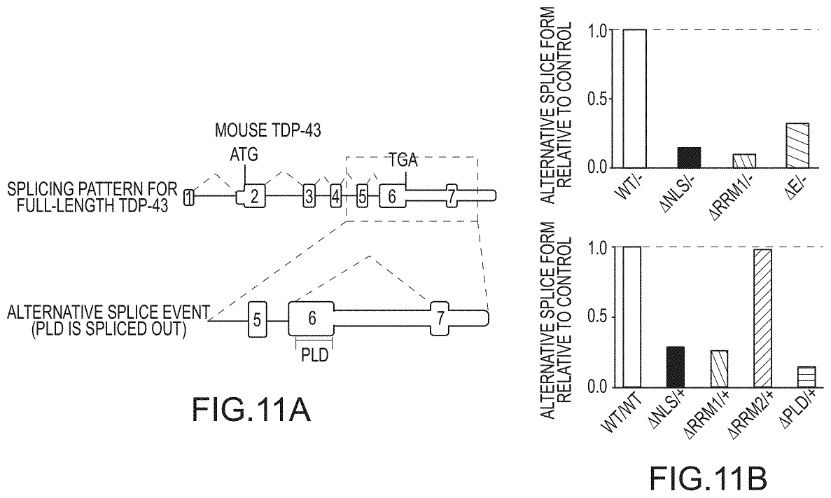

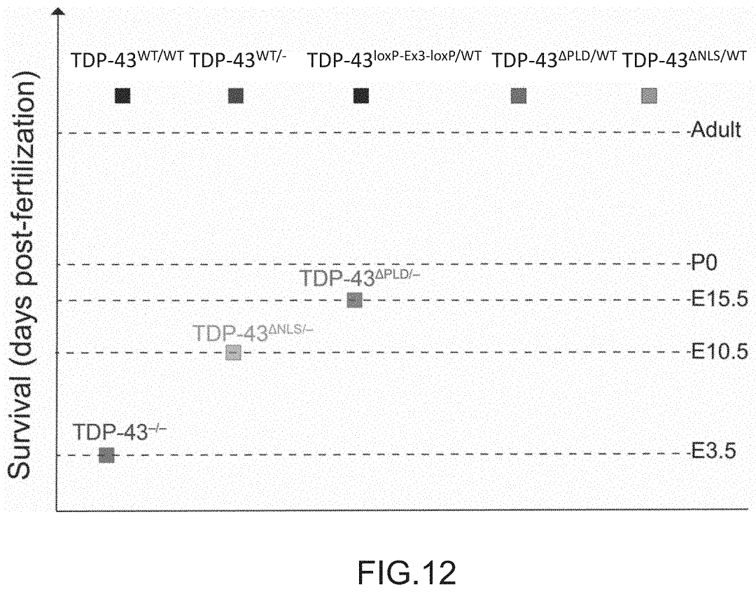

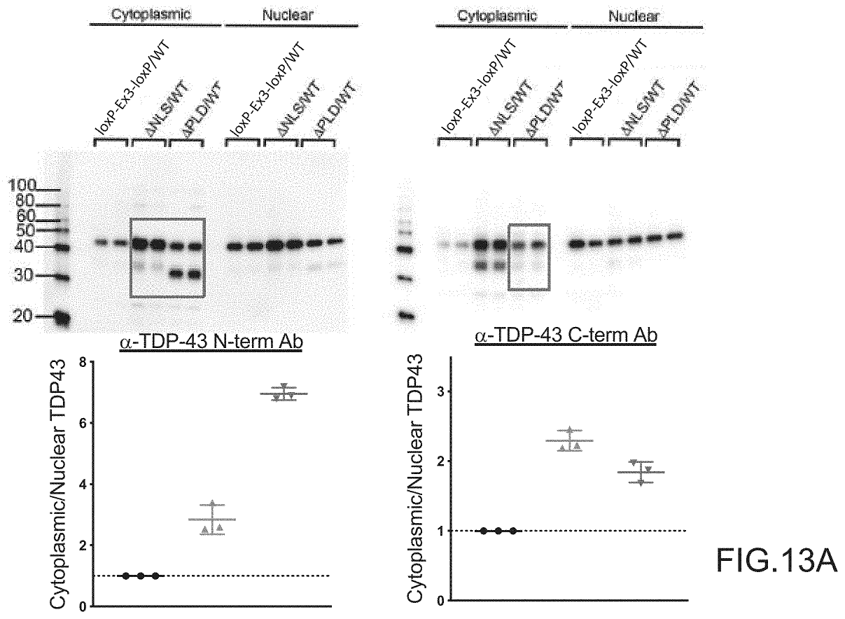

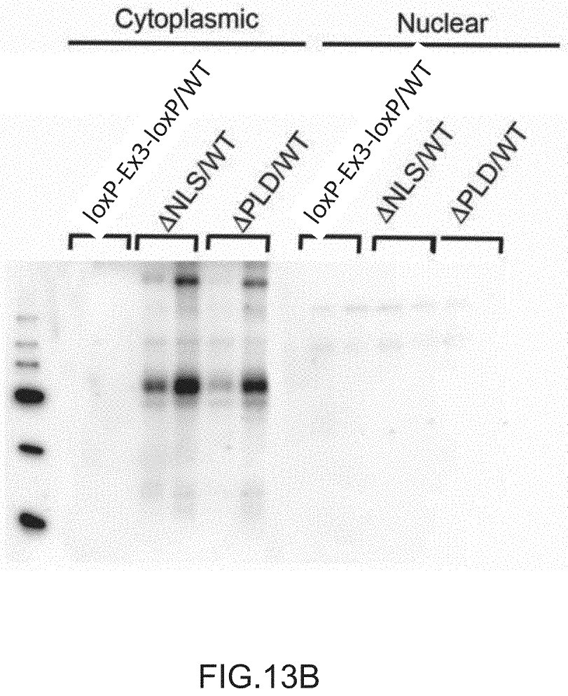

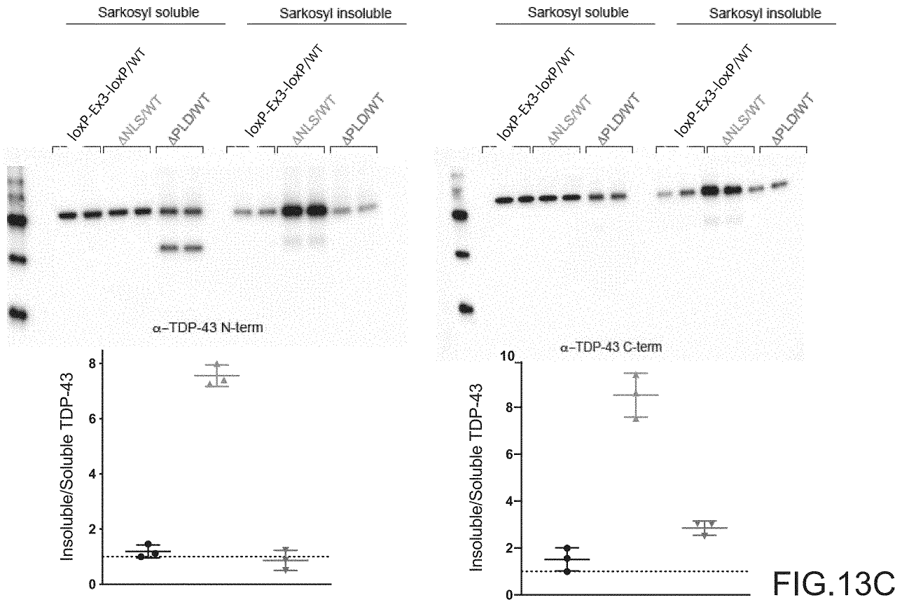

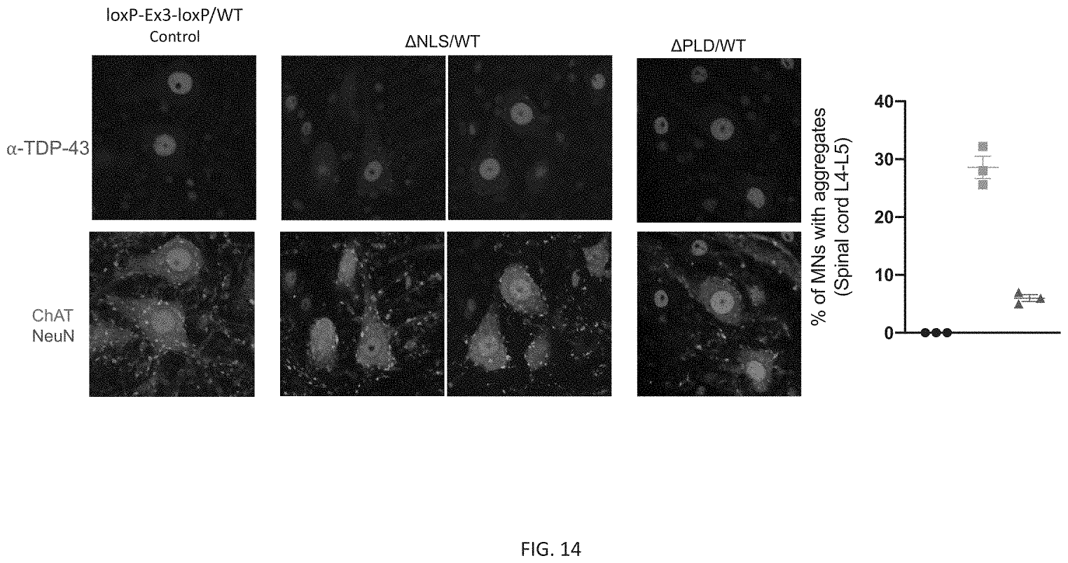

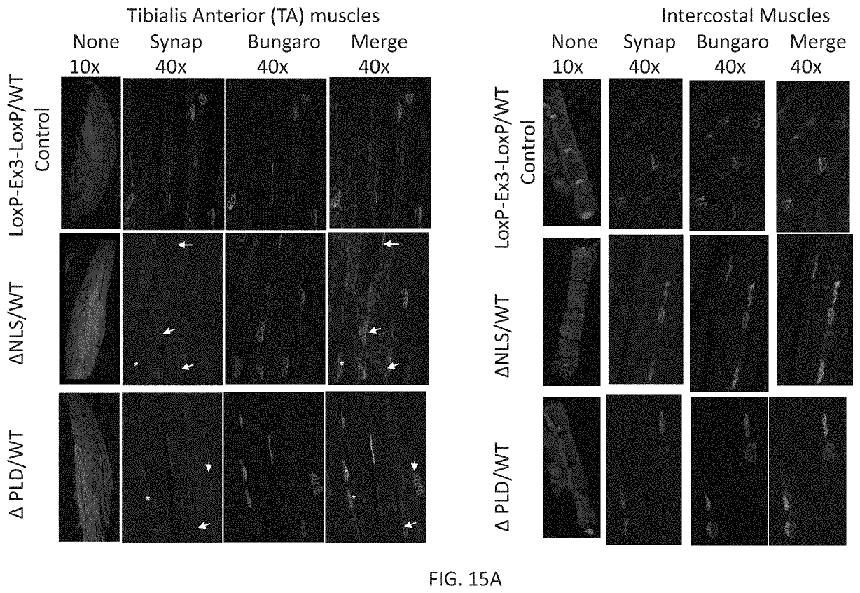

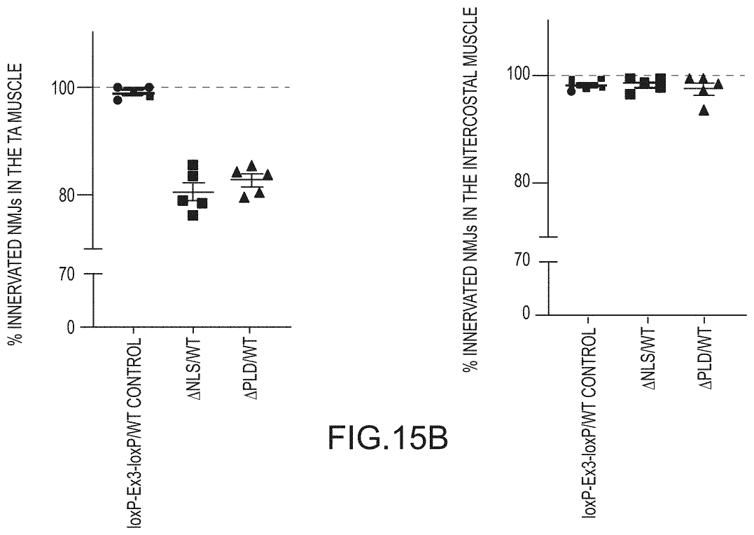

A mutated TARDBP gene may comprise a structure illustrated in FIG. 3A. A mutated TARDBP gene may encode a mutant TDP-43 polypeptide depicted in FIG. 3A.

Methods of Making Cells and Non-Human Animals Comprising and Expressing a Mutant TARDBP Gene

As outlined above, methods and compositions are provided herein to allow for the targeted genetic modification of a TARDBP locus, e.g., for making a cell comprising a mutated TARDBP gene and/or for evaluating the biological function of a TDP-43 structural domain. It is further recognized that additional targeted genetic modification can be made. Such systems that allow for these targeted genetic modifications can employ a variety of components and for ease of reference, herein the term “targeted genomic integration system” generically includes all the components required for an integration event (i.e. the various nuclease agents, recognition sites, insert DNA polynucleotides, targeting vectors, target genomic locus, etc.).

A method of making a non-human animal cell that expresses a mutant TDP-43 polypeptide and/or for evaluating the biological function of a TDP-43 structural domain may comprise modifying the genome of the cell to comprise a mutated TARDBP gene. The mutated TARDBP gene may encode the mutant TDP 43 polypeptide, wherein the mutant TDP-43 polypeptide lacks the functional structural domain.

A method of making a non-human animal cell that expresses a mutant TDP-43 polypeptide and/or for evaluating the biological function of a TDP-43 structural domain may comprise modifying the genome of the cell to comprise a mutated TARDBP gene, wherein the mutated TARDBP gene comprises a knockout mutation.

The methods provided herein comprise introducing into a cell one or more polynucleotides or polypeptide constructs comprising the various components of the targeted genomic integration system. “Introducing” means presenting to the cell the sequence (polypeptide or polynucleotide) in such a manner that the sequence gains access to the interior of the cell. The methods provided herein do not depend on a particular method for introducing any component of the targeted genomic integration system into the cell, only that the polynucleotide gains access to the interior of a least one cell. Methods for introducing polynucleotides into various cell types are known in the art and include, but are not limited to, stable transfection methods, transient transfection methods, and virus-mediated methods.

In some embodiments, the cells employed in the methods and compositions have a DNA construct stably incorporated into their genome. “Stably incorporated” or “stably introduced” means the introduction of a polynucleotide into the cell such that the nucleotide sequence integrates into the genome of the cell and is capable of being inherited by progeny thereof. Any protocol may be used for the stable incorporation of the DNA constructs or the various components of the targeted genomic integration system.

Transfection protocols as well as protocols for introducing polypeptides or polynucleotide sequences into cells may vary. Non-limiting transfection methods include chemical-based transfection methods include the use of liposomes; nanoparticles; calcium phosphate (Graham et al. (1973). Virology 52 (2): 456-67, Bacchetti et al. (1977) Proc Natl Acad Sci USA 74 (4): 1590-4 and, Kriegler, M (1991). Transfer and Expression: A Laboratory Manual. New York: W. H. Freeman and Company. pp. 96-97); dendrimers; or cationic polymers such as DEAE-dextran or polyethylenimine. Non chemical methods include electroporation; Sono-poration; and optical transfection. Particle-based transfections include the use of a gene gun, magnet assisted transfection (Bertram, J. (2006) Current Pharmaceutical Biotechnology 7, 277-28). Viral methods can also be used for transfection.

Cells comprising a mutated TARDBP gene can be generated employing the various methods disclosed herein. Modifying may comprise replacing an endogenous TARDBP gene with the mutated TARDBP gene that encodes the mutant TDP-43 polypeptide and/or replacing an endogenous TARDBP gene with a TARDBP gene comprising a knockout mutation, such as a conditional knockout mutation. Modifying may comprise culturing the cell in conditions that eliminates expression of the TARDBP gene comprising a knockout mutation. Conditions that may eliminate the expression of a TARDBP gene may include expressing a recombinase protein, e.g., cre-recombinase.

Such modifying methods may comprise (1) integrating a mutated TARDBP gene at the target TARDBP genomic locus of interest of a pluripotent cell of a non-human animal to generate a genetically modified pluripotent cell comprising the mutated TARDBP gene in the targeted TARDBP genomic locus employing the methods disclosed herein; and (2) selecting the genetically modified pluripotent cell having the mutated TARDBP gene at the target TARDBP genomic locus. Animals may be further generated by (3) introducing the genetically modified pluripotent cell into a host embryo of the non-human animal, e.g., at a pre-morula stage; and (4) implanting the host embryo comprising the genetically modified pluripotent cell into a surrogate mother to generate an F0 generation derived from the genetically modified pluripotent cell. The non-human animal can be a non-human mammal, a rodent, a mouse, a rat, a hamster, a monkey, an agricultural mammal or a domestic mammal, or a fish or a bird.

The pluripotent cell can be a human ES cell, a non-human ES cell, a rodent ES cell, a mouse ES cell, a rat ES cell, a hamster ES cell, a monkey ES cell, an agricultural mammal ES cell or a domesticated mammal ES cell. In other embodiments, the pluripotent cell is a non-human cell, a mammalian cell, a human cell, a non-human mammalian cell, a human pluripotent cell, a human ES cell, a human adult stem cell, a developmentally-restricted human progenitor cell, a human iPS cell, a rodent cell, a rat cell, a mouse cell, a hamster cell. In one embodiment, the targeted genetic modification results in a mutated TARDBP gene.

A mouse pluripotent cell, totipotent cell, or host embryo can be from any strain of mouse including, for example, inbred strains, hybrid strains, and outbred strains. Examples of mouse strains include a 129 strain, a C57BL strain (e.g., a C57BL/6 strain), a mix of 129 and C57BL/6 (e.g., 50% 129 and 50% C57BL/6), a BALB/c strain, and a Swiss Webster strain. Examples of 129 strains include 129P1, 129P2, 129P3, 129X1, 129S1 (e.g., 12951/SV, 12951/SvIm), 129S2, 129S4, 129S5, 129S9/SvEvH, 129S6 (129/SvEvTac), 129S7, 129S8, 129T1, and 129T2 (see, e.g., Festing et al. (1999) Revised nomenclature for strain 129 mice, Mammalian Genome 10:836). Examples of C57BL strains include C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/01a. Mice can be mixes of an aforementioned 129 strain (e.g., a 129S6 (129/SvEvTac) strain) and an aforementioned C57BL/6 strain, mixes of one or more aforementioned 129 strains, or mixes of one or more aforementioned C57BL strains. Mice can also be from a strain excluding 129 strains.

A rat pluripotent cell, totipotent cell, or host embryo can be from any rat strain, including, for example, inbred strains, hybrid strains, and outbred strains. Examples of rat strains include an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rat pluripotent cells, totipotent cells, or host embryos can also be obtained from a strain derived from a mix of two or more strains recited above. For example, the rat pluripotent cell, totipotent cell, or host embryo can be derived from a strain selected from a DA strain and an ACI strain. The ACI rat strain is characterized as having black agouti, with white belly and feet and an RT1^{avl} haplotype. Such strains are available from a variety of sources including Harlan Laboratories. An example of a rat ES cell line from an ACI rat is the ACI.G1 rat ES cell. The Dark Agouti (DA) rat strain is characterized as having an agouti coat and an RT1^{avl} haplotype. Such rats are available from a variety of sources including Charles River and Harlan Laboratories. Examples of a rat ES cell line from a DA rat and are the DA.2B rat ES cell line or the DA.2C rat ES cell line. Other examples of rat strains are provided, for example, in US 2014/0235933, US 2014/0310828, and US 2014/0309487, each of which is herein incorporated by reference in its entirety for all purposes.

For example, germline-transmittable rat ES cells can be obtained by culturing isolated rat ES cells on a feeder cell layer with a medium comprising N2 supplement, B27 supplement, about 50 U/mL to about 150 U/mL leukemia inhibitory factor (LIF), and a combination of inhibitors consisting of a MEK inhibitor and a GSK3 inhibitor, wherein the feeder cell layer is not modified to express LIF, and wherein the rat ES cells: (i) have been modified to comprise a targeted genetic modification comprising at least one insertion of a heterologous polynucleotide comprising a selection marker into the genome of the rat ES cells and are capable of transmitting the targeted genetic modification through the germline; (ii) have a normal karyotype; (iii) lack expression of c-Myc; and (iv) form spherical, free-floating colonies in culture (See, for example, US 2014-0235933 A1 and US 2014-0310828 A1, each of which is incorporated by reference in its entirety). Other examples of derivation of rat embryonic stem cells and targeted modification are provided, e.g., in Yamamoto et al. (“Derivation of rat embryonic stem cells and generation of protease-activated receptor-2 knockout rats,” Transgenic Res. 21:743-755, 2012) and Kwamata and Ochiya (“Generation of genetically modified rats from embryonic stem cells,” Proc. Natl. Acad. Sci. USA 107(32):14223-14228, 2010).

Nuclear transfer techniques can also be used to generate the non-human animals. Briefly, methods for nuclear transfer include the steps of: (1) enucleating an oocyte; (2) isolating a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods oocytes are generally retrieved from deceased animals, although they may be isolated also from either oviducts and/or ovaries of live animals. Oocytes can be matured in a variety of medium known to those of ordinary skill in the art prior to enucleation. Enucleation of the oocyte can be performed in a number of manners well known to those of ordinary skill in the art. Insertion of the donor cell or nucleus into the enucleated oocyte to form a reconstituted cell is usually by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell is typically activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, are typically cultured in medium well known to those of ordinary skill in the art and then transferred to the womb of an animal. See, for example, US20080092249, WO/1999/005266A2, US20040177390, WO/2008/017234A1, and U.S. Pat. No. 7,612,250, each of which is herein incorporated by reference.

Other methods for making a non-human animal comprising in its germline one or more genetic modifications as described herein is provided, comprising: (a) modifying a targeted genomic TARDBP locus of a non-human animal in a prokaryotic cell employing the various methods described herein; (b) selecting a modified prokaryotic cell comprising the genetic modification at the targeted genomic locus; (c) isolating the genetically modified targeting vector from the genome of the modified prokaryotic cell; (d) introducing the genetically modified targeting vector into a pluripotent cell of the non-human animal to generate a genetically modified pluripotent cell comprising the insert nucleic acid at the targeted TARDBP genomic locus; (e) selecting the genetically modified pluripotent cell; (f) introducing the genetically modified pluripotent cell into a host embryo of the non-human animal at a pre-morula stage; and (g) implanting the host embryo comprising the genetically modified pluripotent cell into a surrogate mother to generate an F0 generation derived from the genetically modified pluripotent cell. In such methods the targeting vector can comprise a large targeting vector. The non-human animal can be a non-human mammal, a rodent, a mouse, a rat, a hamster, a monkey, an agricultural mammal or a domestic mammal. The pluripotent cell can be a human ES cell, a non-human ES cell, a rodent ES cell, a mouse ES cell, a rat ES cell, a hamster ES cell, a monkey ES cell, an agricultural mammal ES cell or a domestic mammal ES cell. In other embodiments, the pluripotent cell is a non-human cell, a mammalian cell, a human cell, a non-human mammalian cell, a human pluripotent cell, a human ES cell, a human adult stem cell, a developmentally-restricted human progenitor cell, a human iPS cell, a human cell, a rodent cell, a rat cell, a mouse cell, a hamster cell. In one embodiment, the targeted genetic modification results in a mutated TARDBP gene, e.g., a mutant TARDBP gene that encodes a mutant TDP-43 polypeptide lacking a functional structural domain and/or a mutant TARDBP gene comprising a knockout mutation

In further methods, the isolating step (c) further comprises (c1) linearizing the genetically modified targeting vector (i.e., the genetically modified LTVEC). In still further embodiments, the introducing step (d) further comprises (d1) introducing a nuclease agent into the pluripotent cell to facilitate homologous recombination. In one embodiment, selecting steps (b) and/or (e) are carried out by applying a selectable agent as described herein to the prokaryotic cell or the pluripotent cell. In one embodiment, selecting steps (b) and/or (e) are carried out via a modification of allele (MOA) assay as described herein.

In some embodiments, various genetic modifications of the target genomic loci described herein can be carried out by a series of homologous recombination reactions (BHR) in bacterial cells using an LTVEC derived from Bacterial Artificial Chromosome (BAC) DNA using VELOCIGENE® genetic engineering technology (see, e.g., U.S. Pat. No. 6,586,251 and Valenzuela, D. M. et al. (2003), Nature Biotechnology 21(6): 652-659, which is incorporated herein by reference in their entireties).

In some embodiments, the targeted pluripotent and/or totipotent cells comprising various genetic modifications as described herein are used as insert donor cells and introduced into a pre-morula stage embryo from a corresponding organism, e.g., an 8-cell stage mouse embryo, via the VELOCIMOUSE® method (see, e.g., U.S. Pat. Nos. 7,576,259, 7,659,442, 7,294,754, and US 2008-0078000 A1, all of which are incorporated by reference herein in their entireties). The non-human animal embryo comprising the genetically modified pluripotent and/or totipotent cells is incubated until the blastocyst stage and then implanted into a surrogate mother to produce an F0 generation. In some embodiments, targeted mammalian ES cells comprising various genetic modifications as described herein are introduced into a blastocyst stage embryo. Non-human animals bearing the genetically modified genomic locus (i.e. a TARDBP locus) can be identified via modification of allele (MOA) assay as described herein. The resulting F0 generation non-human animal derived from the genetically modified pluripotent and/or totipotent cells is crossed to a wild-type non-human animal to obtain F1 generation offspring. Following genotyping with specific primers and/or probes, F1 non-human animals that are heterozygous for the genetically modified genomic locus are crossed to each other to produce F2 generation non-human animal offspring that are homozygous for the genetically modified genomic locus.

In one embodiment, a method for making a cell comprising a mutated TARDBP gene is provided. Such a method comprising: (a) contacting a pluripotent cell with a targeting construct comprising a mutated TARDBP gene or a mutated portion thereof flanked by 5′ and 3′ homology arms; wherein the targeting construct undergoes homologous recombination with the TARDBP locus in a genome of the cell to form a modified pluripotent cell. Methods of making a non-human animal further comprises (b) introducing the modified pluripotent cell into a host embryo; and (c) gestating the host embryo in a surrogate mother, wherein the surrogate mother produces progeny comprising a modified TARDBP locus, wherein said genetic modification results in a mutant TDP-43 polypeptide lacking a functional structural domain.

In some embodiments, a cell comprising a mutated TARDBP gene may be made by modifying an ES cell to comprise the mutated TARDB gene and culturing in vitro the ES cell in differentiating medium. In some embodiments, culturing in vitro the ES cell comprises differentiating the ES cell into primitive ectoderm cells or embryonic stem cell derived motor neurons (ESMNs).

Cells and Animals

The cells (which may be comprised within non-human animal tissues or non-human animals) disclosed herein may be any type of cell comprising a mutated TARDBP gene as disclosed herein. A cell may comprise a mutated non-human animal TARDBP gene (e.g., a mutated TARDBP gene of the non-human animal) or a mutated human TARDBP gene.

A cell may comprise a mutated TARDBP gene that encodes a mutant TDP-43 polypeptide, wherein the mutant TDP-43 polypeptide lacks a functional structural domain, and wherein the cell expresses the mutant TDP-43 polypeptide. For example, a cell may comprise a mutated TARDBP gene that encodes a mutant TDP-43 polypeptide lacking a functional structural domain comprising the nuclear localization signal (NLS), the RNA recognition motif 1 (RRM1), the RNA recognition motif 2 (RRM2), the putative nuclear export signal (E), the prion like domain (PLD), or a combination thereof. A cell may comprise a mutated TARDBP gene that encodes a mutant TDP-43 polypeptide lacking a functional structural domain due to one or more of the following: (a) a point mutation of an amino acid in the NLS (e.g., K82A K83A, R84A, K95A, K97A, K98A or a combination thereof), (b) a point mutation of an amino acid in RRM1 (e.g., F147L and/or F149L) (c) a point mutation of an amino acid in the RRM2 (F194L and/or F229L), (d) a deletion of at least a portion of the nuclear export signal (e.g., a deletion of the amino acids at and between positions 239 and 250 of a wildtype TDP-43 protein), and (e) a deletion of at least a portion of the prion-like domain (e.g., a deletion of the amino acids at and between positions 274 and 414 of a wildtype TDP-43 polypeptide). A cell may comprise a mutated TARDBP gene that encodes a mutant TDP-43 polypeptide comprising the following mutations: K82A K83A, R84A, K95A, K97A, and K98A, wherein the mutant TDP-43 polypeptide lacks a functional NLS. A cell may comprise a mutated TARDBP gene that encodes a mutant TDP-43 polypeptide comprising a deletion between and including the amino acids at positions 274 to 414 of a wildtype TDP-43 polypeptide, wherein the mutant TDP-43 polypeptide lacs a functional PLD. A cell may comprise a mutated TARDBP gene that encodes a mutant TDP-43 polypeptide comprising the point mutations F147L and F149L, wherein the mutant TDP-43 polypeptide lacks a functional RRM1. A cell may comprise a mutated TARDBP gene that encodes a mutant TDP-43 polypeptide comprising the point mutations F194L and F229L, wherein the mutant polypeptide lacks a functional RRM2. A cell may comprise a mutated TARDBP gene that encodes a mutant TDP-43 polypeptide comprising a deletion of the nuclear export signal between and including the amino acids at positions 239 and 250 of a wildtype TDP-43 polypeptide, wherein the mutant TDP-43 polypeptide lacks a functional E.

A cell may comprise a mutated TARDBP gene comprising a knockout mutation, e.g., a conditional knockout mutation, a deletion of the entire coding sequence of the TARDBP gene, etc. A cell may comprise a mutated TARDBP gene comprising a conditional knockout mutation, e.g., the mutated TARDBP gene may comprise site-specific recombination recognition sequence, e.g., a loxp sequence. A cell may comprise a mutated TARDBP gene comprising a loxp sequence flanking an exon comprising a TDP-43 coding sequence, e.g. exon 3. A cell may comprise a mutated TARDBP gene comprising a loxp sequence and lacking a TDP-43 coding sequence, e.g., exon 3. A cell may comprise a mutated TARDBP gene lacking the entire TDP-43 coding sequence, e.g., a mutated TARDBP gene comprising a deletion of the entire coding sequence of a TDP-43 polypeptide.

In some embodiments, the cell may comprise the mutated TARDBP gene inserted at the endogenous TARDBP locus, e.g., in its germline genome. In some embodiments, a cell comprises a mutated TARDBP gene, e.g., mutated TARDBP gene comprising a knockout mutation and/or a mutated TARDBP gene that encodes a mutant TDP-43 polypeptide, that replaces an endogenous TARDBP gene at an endogenous TARDBP locus. In some embodiments, a mutated TARDBP gene is operably linked to an endogenous TARDBP promoter and/or regulatory element.

The cells may be heterozygous or homozygous for a mutated TARDBP gene. A diploid organism has two alleles, one at each genetic locus of the pair of homologous chromosomes. Each pair of alleles represents the genotype of a specific genetic locus. Genotypes are described as homozygous if there are two identical alleles at a particular locus and as heterozygous if the two alleles differ.

A cell may comprise (i) at an endogenous TARDBP locus, a replacement of an endogenous TARDBP gene with a mutated TARDBP gene that encodes a mutant TDP-43 polypeptide, and (ii) at the other endogenous TARDPP locus of a homologous chromosome, a mutated TARDBP gene comprising a knockout mutation.

A cell comprising a mutated TARDBP gene may express the mutant TDP-43 polypeptide encoded therefrom. A cell comprising a mutated TARDBP gene and expressing a mutant TDP-43 polypeptide encoded therefrom may, or may not, express a wildtype TDB-43 polypeptide.

A cell comprising a mutated TARDBP gene may express the mutant TDP-43 polypeptide encoded therefrom and may be characterized by one or more of the following (i) a level of mRNA transcripts of the mutated TARDBP gene that is comparable to the level of mRNA transcript levels of a wildtype TARDBP gene in a control cell, (ii) increased levels of the mutant TDP-43 polypeptide compared to levels of wildtype TDP-43 polypeptide in a control cell, (iii) the mutant TDP-43 polypeptide is found at a higher concentration in the cytoplasm than in the nucleus of the cell, (iv) the mutant TDP-43 polypeptide exhibits increased insolubility compared to a wildtype TDP-43 polypeptide, (v) cytoplasmic aggregates comprising the mutant TDP 43 polypeptide, (vi) increased splicing of cryptic exons of genes compared to that of cells expressing a wildtype TDP-43, (vii) decreased levels of an alternatively spliced TDP-43 mRNA lacking a sequence encoding a TDP-43 PLD.

The cells may be cultured in vitro, may be examined ex vivo, or in vivo. For example, the cells can be in vivo within an animal.

The cells may be eukaryotic cells, which include, for example, fungal cells (e.g., yeast), plant cells, animal cells, mammalian cells, non-human mammalian cells, and human cells. The term “animal” includes any member of the animal kingdom, including, for example, mammals, fishes, reptiles, amphibians, birds, and worms. A mammalian cell can be, for example, a non-human mammalian cell, a rodent cell, a rat cell, a mouse cell, or a hamster cell. Other non-human mammals include, for example, non-human primates, monkeys, apes, orangutans, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. In some embodiments, an animal can be a human or a non-human animal, including, but not limited to, mice, rats, rabbits, dogs, cats, pigs, and non-human primates, including, but not limited to, monkeys and chimpanzees. In some embodiments, a non-human animal cell is a rodent cell, e.g., a rat cell or a mouse cell.

Non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 12959/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{avl} haplotype. Such strains are available from a variety of sources including Harlan Laboratories. The Dark Agouti (DA) rat strain is characterized as having an agouti coat and an RT1^{avl} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

The cells can also be any type of undifferentiated or differentiated state. For example, a cell may be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells may also be derived from an ES cell. For example, the cells can be neuronal cells (e.g., ES-cell-derived motor neurons (ESMNs), primitive ectoderm-like cells, embryoid body cells, etc.

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

Methods Employing a System Expressing a Mutant TDP-43 Polypeptide

Cells and non-human animals comprising a mutated TARDBP gene and expressing a mutant TDP-43 polypeptide lacking a functional structural domain encoded therefrom as described herein (and tissues or animals comprising such cells) provide a model for studying the function of structural domains of TDP-43 and/or TDP-43 proteinopathies. For example, cells or non-human animals comprising a mutated TARDBP gene and expressing a mutant TDP-43 polypeptide encoded therefrom lacking a functional structural domain may exhibit phenotypes characteristic of TDP-43 proteinopathy. In some embodiments, cells, e.g., (a) embryonic stem cell derived motor neurons (ESMNs) comprising a mutated TARDBP gene and expressing a mutant TDP-43 polypeptide encoded therefrom lacking a functional structural domain and/or (b) isolated from non-human animals comprising at an endogenous TARDBP locus a replacement of the endogenous TARDBP gene with a mutated TARDBP gene and expressing a mutant TDP-43 polypeptide therefrom, may be characterized by one or more of the following (i) a level of mRNA transcripts of the mutated TARDBP gene that is comparable to the level of mRNA transcript levels of a wildtype TARDBP gene in a control cell, (ii) increased levels of the mutant TDP-43 polypeptide compared to levels of wildtype TDP-43 polypeptide in a control cell, (iii) the mutant TDP-43 polypeptide is found at a higher concentration in the cytoplasm than in the nucleus of the cell, (iv) the mutant TDP-43 polypeptide exhibits increased insolubility compared to a wildtype TDP-43 polypeptide, (v) cytoplasmic aggregates comprising the mutant TDP 43 polypeptide, (vi) increased splicing of cryptic exons of genes compared to that of cells expressing a wildtype TDP-43, (vii) decreased levels of an alternatively spliced TDP-43 mRNA lacking a sequence encoding a TDP-43 PLD.

Thus, cells comprising a mutated TARDBP gene and expressing a mutant TDP-43 polypeptide lacking a functional structural domain encoded therefrom as described herein (and tissues or animals comprising such cells) also provide a system for identifying a therapeutic candidate agent for treating, preventing and/or inhibiting one or more symptoms of TDP-43 proteinopathy (e.g., cytoplasmic accumulation of the mutant TDP-43 polypeptide) and/or restoring the biological functions of a wildtype TDP-43 polypeptide (e.g., repression of cryptic exon splicing and/or increasing the levels of the alternative spliced TDP-43 mRNA). In some embodiments, an effect of a therapeutic agent is determined by contacting a cell comprising a mutated TARDBP gene and expressing a mutant TDP-43 polypeptide lacking a functional structural domain encoded therefrom with the therapeutic candidate agent. Contacting may be performed in vitro. Contacting may comprise administering to an animal the therapeutic candidate agent.

In some embodiments, performing an assay includes determining the effect on the phenotype and/or genotype of cell or animal contacted with the drug. In some embodiments, performing an assay includes determining lot-to-lot variability for a drug (In some embodiments, performing an assay includes determining the differences between the effects on a cell or animal described herein contacted with the drug administered and a control cell or animal (e.g., expressing a wildtype TDP-43).

Exemplary parameters that may be measured in non-human animals (or in and/or using cells isolated therefrom) for assessing the pharmacokinetic properties of a drug include, but are not limited to, agglutination, autophagy, cell division, cell death, complement-mediated hemolysis, DNA integrity, drug-specific antibody titer, drug metabolism, gene expression arrays, metabolic activity, mitochondrial activity, oxidative stress, phagocytosis, protein biosynthesis, protein degradation, protein secretion, stress response, target tissue drug concentration, non-target tissue drug concentration, transcriptional activity, and the like.

Oligonucleotides for Selectively Decreasing Full-Length TDP-43 mRNA

FIG. 11A illustrates the full-length TDP-43 pre-mRNA, and the normal (top panel) and alternative (bottom panel) splice events that occur at its 3′end. As shown, exon 6 encodes the prion-like domain (PLD) in the full-length TDP-43 protein formed with the normal splice event, whose coding sequence terminates at the end of the PLD. Two new exons (7 and 8) are formed by an alternative splicing event from one of at least three alternative 5′-splice site within exon 6 to a downstream alternative 3′-splice site, e.g., adjacent to putative exon 7. There is evidence for a second alternative splice event from alternative exon 7 to alternative exon 8.

In the mouse, alternative 5′-splice sites within or at the beginning of exon 6 described herein are mapped to the following positions: (a) chromosome 4:148,618,647; (b) chromosome 4:148,618,665; and (c) chromosome 4:148,618,674 in a mouse. The alternative 3′-splice site in exon 7 is mapped to position chromosome 4: 148,617,705. The second alternative splice event from exon 7 to exon 8 occurs from chromosome 4: 148,617,566 to chromosome 4: 148,616,844. A skilled artisan would be able to determine similar alternative 5′ and 3′ splice sites in other TARDBP genes, e.g., human TARDBP genes.

Alternative splicing from an alternative 5′-splice site within exon 6 to a downstream alternative 3′-splice site is predicted to produce an mRNA with most of the PLD coding sequence being replaced with a sequence encoding a TDP-43 polypeptide lacking a PLD. For example, alternative splicing from any one of (a) chromosome 4:148,618,647; (b) chromosome 4:148,618,665; and (c) chromosome 4:148,618,674 to chromosome 4: 148,617,705 (and any corresponding position in the human TARDBP gene) may produce an mRNA with most of the PLD coding sequence being replaced with an alternative mRNA predicted to encode a truncated form of TDP-43 lacking a PLD, in which the PLD is replaced with 18 amino acids. This second alternative splicing event does not produce any new forms of TDP-43 protein because the open reading frame stops in exon 7 upstream of the exon 7 5′-splice site.

The observation that TDP-43 lacking the PLD can support viability, especially in motor neurons, and the decreased levels of this alternative spliced TDP-43 mRNA in cells expressing ΔPLD or ΔNLS mutated TARDBP genes, along with their ALS-like phenotype, suggests that this alternative spliced TDP-43 mRNA and its translated truncated product may not contribute to, and may be protective against, TDP-43 proteinopathies. The application of siRNAs, antisense oligonucleotides and/or CRISPR/Cas9 systems designed to ablate or inactivate TDP-43 mRNA isoforms that encode forms of the protein containing the PLD could deplete variants of TDP-43 that are prone to pathological aggregation while sparing the alternatively spliced mRNA that produces the truncated TDP-43 protein without the PLD. The truncated form of TDP-43 might be resistant to pathological aggregation while still supporting cellular life, especially the viability of motor neurons.

Accordingly, a therapeutic strategy would consist of finding active antisense oligonucleotides (ASOs) or siRNAs that target only those TDP-43 mRNA sequences comprising a sequence that encodes a PLD, e.g., those mRNA comprising a sequence encoded by a genomic sequence subsequent to an alternative splice site within exon 6. As a non-limiting example, ASOs or siRNAs may target those mRNAs which comprise sequences transcribed from a TARDBP gene after the codon(s) that encode an alternative 5′ splice site that results in the splicing out of a PLD domain. ASOs or siRNAs designed to target this region of a TDP-43 mRNA will recognize only the full-length TDP-43 mRNAs that encode TDP-43 polypeptides comprising a PLD while sparing the alternatively spliced TDP-43 mRNA that encodes the truncated and potentially protected TDP-43 polypeptides lacking a PLD. In other words, such ASOs or siRNAs should not be able to recognize or enhance degradation of the alternatively spliced TDP-43 mRNA. ASOs or siRNAs may target a TDP-43 mRNA sequence coding for amino acids 287-414 of a TDP-43 polypeptide or any 3′ untranslated region upstream of the 3′ alternative splice site of exon 7. An ASO may promote degradation of the mRNA by RNaseH-mediated cleavage, for example via a -5-10-5 gapmer. An siRNA may promote mRNA degradation and or protein synthesis by RNA interference.

Another therapeutic strategy would be the application of a CRISPR/Cas system to selectively target and delete a genomic sequence spanning an alternative 5′ splice site within exon 6 of a TARDBP gene and downstream 3′ splice site, e.g., at exon 7. In this way, only mRNA encoding a truncated TDP-43 polypeptide lacking a PLD may be transcribed.

A. Antisense Oligonucleotides and siRNA

Antisense oligonucleotides (ASOs) and small interfering RNA (siRNA) that target sequences within a pre-mRNA may enhance degradation of undesirable isoforms. As designed herein, ASOs or siRNAs may be used to destroy TDP-43 mRNA encoding a PLD while sparing the alternatively spliced TDP-43 mRNA. To reduce the levels of only the full-length TDP-43 mRNA, ASOs or siRNAs may target a TDP-43 mRNA comprising a sequence between an alternative 5′ splice site within exon 6 to (ii) a downstream alternative 3′ splice site, e.g., a TDP-43 mRNA comprising a sequence coding for amino acids 287-414 of a TDP-43 polypeptide and/or any 3′ untranslated region upstream of the alternative splice site. See, FIG. 11A. In some embodiments, the alternative 5′ splice site within exon 6 correlates to a TARDBP genomic position selected from the group consisting of (a) mouse chromosome 4:148,618,647; (b) mouse chromosome 4:148,618,665; (c) mouse chromosome 4:148,618,674, and (d) any corresponding position in a human TARDBP gene. In some embodiments, the downstream alternative 3′ splice site correlates to a mouse chromosome 4: 148,617,705 or a corresponding position in a human TARDBP gene.

Antisense oligonucleotides or siRNAs targeted to a TDP-43 mRNA sequence encoding a PLD may have chemically modified subunits arranged in patterns, or motifs, to confer to the antisense oligonucleotides properties such as enhanced inhibitory activity, increased binding affinity for a target nucleic acid, or resistance to degradation by in vivo nucleases.

Antisense oligonucleotides typically contain at least one region modified so as to confer increased resistance to nuclease degradation, increased cellular uptake, increased binding affinity for the target nucleic acid, and/or increased inhibitory activity. A second region of a antisense oligonucleotides may optionally serve as a substrate for the cellular endonuclease RNase H, which cleaves the RNA strand of an RNA:DNA duplex.

In certain embodiments, the antisense oligonucleotides are uniform sugar-modified oligonucleotides. Antisense oligonucleotides may comprise a gapmer motif. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE and 2′-O—CH3, among others), and bicyclic sugar modified nucleosides. In certain embodiments, wings may include several modified sugar moieties, including, for example 2′-MOE. In certain embodiments, wings may include several modified and unmodified sugar moieties. In certain embodiments, wings may include various combinations of 2′-MOE nucleosides and 2′-deoxynucleosides.

Each distinct region may comprise uniform sugar moieties, variant, or alternating sugar moieties. The wing-gap-wing motif is frequently described as “X-Y-Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides.“Y” may comprise 2′-deoxynucleosides. As used herein, a gapmer described as “X-Y-Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, Y is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30, or more nucleosides. Thus, gapmers described herein include, but are not limited to, for example, 5-10-5, 5-10-4, 4-10-4, 4-10-3, 3-10-3, 2-10-2, 5-9-5, 5-9-4, 4-9-5, 5-8-5, 5-8-4, 4-8-5, 5-7-5, 4-7-5, 5-7-4, or 4-7-4.

An antisense oligonucleotide targeted to a TDP-43 mRNA sequence encoding a PLD may possess a 5-10-5 gapmer motif.

An antisense oligonucleotide targeted to a TDP-43 mRNA sequence encoding a PLD may comprise a gap-narrowed motif. A gap-narrowed antisense oligonucleotide targeted to a TDP-43 mRNA may have a gap segment of 9, 8, 7, or 6 2′-deoxynucleotides positioned immediately adjacent to and between wing segments of 5, 4, 3, 2, or 1 chemically modified nucleosides. A chemically modified nucleoside may comprise a bicyclic sugar. A bicyclic sugar may comprise a 4′ to 2′ bridge selected from among: 4′-(CH2)n-O-2′ bridge, wherein n is 1 or 2; and 4′-CH2-O—CH2-2′. A bicyclic sugar may comprise a 4′-CH(CH3)-O-2′ bridge. A chemical modification may comprise a non-bicyclic 2′-modified sugar moiety, e.g., a 2′-O-methylethyl group or a 2′-O-methyl group. In some embodiments, an antisense oligonucleotide comprising a gapmer motif targeting a TDP-43 mRNA sequence between alternative 5′ and 3′ splice sites, wherein the alternative 5′ splice site is within exon 6, e.g., wherein the alternative 5′ splice site correlates to a TARDBP genomic position selected from the group consisting of (a) mouse chromosome 4:148,618,647; (b) mouse chromosome 4:148,618,665; (c) mouse chromosome 4:148,618,674, and (d) any corresponding position in a human TARDBP gene and wherein the alternative 3′ splice junction correlates to a TARDBP genomic position of chromosome 4: 148,617,705. In some embodiments, an siRNA comprises a sequence targeting a TDP-43 mRNA sequence between alternative 5′ and 3′ splice sites, wherein the alternative 5′ splice site is within exon 6, e.g., wherein the alternative 5′ splice site correlates to a TARDBP genomic position selected from the group consisting of (a) mouse chromosome 4:148,618,647; (b) mouse chromosome 4:148,618,665; (c) mouse chromosome 4:148,618,674, and (d) any corresponding position in a human TARDBP gene and wherein the alternative 3′ splice junction correlates to a TARDBP genomic position of chromosome 4: 148,617,705.

An antisense oligonucleotide or siRNAs targeted to a TDP-43 mRNA sequence encoding a PLD may be uniformly modified. In certain embodiments, each nucleoside is chemically modified. In certain embodiments, the chemical modification comprises a non-bicyclic 2′-modified sugar moiety. In certain embodiments, the 2′-modified sugar moiety comprises a 2′-O-methoxyethyl group. In certain embodiments, the 2′-modified sugar moiety comprises a 2′-O-methyl group.

ASOs or siRNAs may also be covalently linked to one or more moieties or conjugates that enhance the activity, cellular distribution, or cellular uptake of the resulting ASOs or siRNAs. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

ASOs or siRNAs may also be modified to have one or more stabilizing groups that are generally attached to one or both termini. Included in stabilizing groups are cap structures. These terminal modifications protect the ASO or siRNAs having terminal nucleic acid from exonuclease degradation and can help in delivery and/or localization within a cell. The cap can be present at the 5′-terminus (5′-cap), or at the 3′-terminus (3′-cap), or can be present on both termini. Cap structures are well known and include, for example, inverted deoxy a basic caps.

ASOs or siRNAs may be any length suitable for binding to a target nucleic acid (e.g., a TDP-43 pre-mRNA) and having the desired effect. For example, an ASO can be about 12 to about 30, about 12 to about 24, about 13 to about 23, about 14 to about 22, about 15 to about 21, about 16 to about 20, about 17 to about 19, or about 18 nucleosides in length. As another example, the ASO can be about 8 to about 80, about 12 to about 50, about 15 to about 30, about 18 to about 24, about 19 to about 22, or about 20 linked nucleosides. Alternatively, the ASOs can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, or about 80 linked nucleosides in length. For example, the ASO can consist of about 15, about 16, about 17, about 18, about 19, about 20, about 21, about 22, about 23, about 24, or about 25 linked nucleosides. In a specific example, an ASO can be about 15 to about 25 linked nucleosides.

The ASOs or siRNAs can be complementary to and/or specifically hybridizable to a target nucleic acid (e.g., a TDP-43 pre-mRNA, e.g., an mRNA sequence encoding a PLD). An ASO and a target nucleic acid are complementary to each other when a sufficient number of nucleobases of the ASO can hydrogen bond with the corresponding nucleobases of the target nucleic acid, such that a desired effect will occur. Specifically hybridizable refers to an ASO having a sufficient degree of complementarity between the ASO and a target nucleic acid to induce a desired effect, while exhibiting minimal or no effects on non-target nucleic acids under conditions in which specific binding is desired (e.g., under physiological conditions).

Some ASOs or siRNAs are at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% complementary to an equal length portion of a TDP-43 pre-mRNA. Alternatively, an ASO can be about 100% complementary to an equal length portion of a TDP-43 pre-mRNA. Percent complementarity of an ASO with a target nucleic acid can be determined using routine methods. For example, an ASO in which 18 of 20 nucleobases of the ASO are complementary to a target region, and would therefore specifically hybridize, would represent 90 percent complementarity. Percent complementarity of an ASO with a region of a target nucleic acid can be determined routinely using BLAST programs (basic local alignment search tools) and PowerBLAST programs that are well-known (see, e.g., Altschul et al. (1990) J. Mol. Biol. 215:403 410 and Zhang and Madden (1997) Genome Res. 7:649-656). Percent homology, sequence identity, or complementarity can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

Non-complementary nucleobases between an ASO or siRNAs and a TDP-43 pre-mRNA may be tolerated provided that the ASO or siRNAs remains able to specifically hybridize to a target nucleic acid. Moreover, an ASO or siRNA may hybridize over one or more segments of a TDP-43 pre-mRNA such that intervening or adjacent segments are not involved in the hybridization event (e.g., a loop structure, mismatch or hairpin structure). The location of a non-complementary nucleobase may be at the 5′ end or 3′ end of the ASO or siRNAs. Alternatively, the non-complementary nucleobase or nucleobases may be at an internal position of the ASO or siRNAs. When two or more non-complementary nucleobases are present, they may be contiguous (i.e., linked) or non-contiguous.

B. Deleting a Genomic Sequence Encoding a TDP-43 PLD

As shown herein, cells remain viable despite expressing only mutant TDP-43 polypeptides lacking a functional PLD. Also described herein is a Clustered Regularly Interspersed Short Palindromic Repeats (CRISPR)/CRISPR-associated (Cas) system, or one or more components of a CRISPR/Cas system, which may be used to delete from a cell, e.g., an embryonic stem cell, a protein like domain (or portion thereof) at an endogenous TARDBP locus as described herein. A CRISPR/Cas system may delete from a cell, e.g., an embryonic stem cell, the genomic sequence encoding for a TDP-43 PLD, e.g., at or near an alternative 5′ splice site within exon 6 through a downstream alternative splice site, e.g., a 3′ splice site of within exon 7. Such components include, for example, Cas proteins and/or guide RNAs (gRNAs), which gRNA may include two separate RNA molecules; e.g., targeter-RNA (e.g., CRISPR RNAs (crRNA) and activator RNA (e.g., tracrRNAs); or a single-guide RNA (e.g., single-molecule gRNA (sgRNA)). In some embodiments, a CRISPR/Cas system comprises a Cas9 protein and at least one gRNA, wherein the gRNA recognizes a sequence at or near a TARDBP genomic position selected from the group consisting of (a) chromosome 4:148,618,647; (b) chromosome 4:148,618,665; (c) chromosome 4:148,618,674, (d) chromosome 4: 148,617,705 and a combination thereof.

CRISPR/Cas systems include transcripts and other elements involved in the expression of, or directing the activity of, Cas genes. A CRISPR/Cas system can be, for example, a type I, a type II, or a type III system. Alternatively, a CRISPR/Cas system can be a type V system (e.g., subtype V-A or subtype V-B). Sequences encoding a TDP-43 prion like domain (or portion thereof), or sequences between the 5′ alternative splice junction (e.g., sequences encoding amino acid 288) and the 3′ alternative splice junction (e.g., adjacent to alternative exon 7), at an endogenous TARDBP locus as described herein may be deleted by utilizing CRISPR complexes (comprising a guide RNA (gRNA) complexed with a Cas protein) for site-directed cleavage of nucleic acids.

A CRISPR/Cas system as described herein may comprise a Cas protein (e.g., Cas1, Cas1B, Cas2, Cas3, Cas4, Cas5, Cas5e (CasD), Cash, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cu1966, and homologs or modified versions thereof) and/or one or more guide RNA (gRNA), which target(s) a gRNA recognition sequence. A CRISPR/Cas system as described herein may further comprise at least one expression construct, which comprises a nucleic acid encoding a Cas protein (e.g., which may be operably linked to a promoter) and/or DNA encoding a gRNA.

Site-specific binding and cleavage of a TARDBP gene by Cas proteins can occur at locations determined by both (i) base-pairing complementarity between the gRNA and the target DNA and (ii) a short motif, called the protospacer adjacent motif (PAM), in the target DNA. The PAM can flank the guide RNA recognition sequence. Optionally, the guide RNA recognition sequence can be flanked on the 3′ end by the PAM. Alternatively, the guide RNA recognition sequence can be flanked on the 5′ end by the PAM. For example, the cleavage site of Cas proteins can be about 1 to about 10 or about 2 to about 5 base pairs (e.g., 3 base pairs) upstream or downstream of the PAM sequence. In some cases (e.g., when Cas9 from S. pyogenes or a closely related Cas9 is used), the PAM sequence of the non-complementary strand can be 5′-N₁GG-3′, where N₁ is any DNA nucleotide and is immediately 3′ of the guide RNA recognition sequence of the non-complementary strand of the target DNA. As such, the PAM sequence of the complementary strand would be 5′-CCN₂-3′, where N₂ is any DNA nucleotide and is immediately 5′ of the guide RNA recognition sequence of the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A.

As disclosed herein, guide RNAs may be provided in any form. In some embodiments, gRNA can be provided in the form of RNA, either as two molecules (a separate crRNA and tracrRNA) or as one molecule (sgRNA), and optionally in the form of a complex with a Cas protein. The gRNA can also be provided in the form of DNA encoding the gRNA. In some embodiments, the DNA encoding the gRNA can encode a single RNA molecule (sgRNA) or separate RNA molecules (e.g., separate crRNA and tracrRNA) (wherein the separate RNA molecules may be provided as one DNA molecule, or as separate DNA molecules encoding the crRNA and tracrRNA, respectively).

In one embodiment, a CRISPR/Cas system as described herein comprises Cas9 protein or a protein derived from a Cas9 from a type II CRISPR/Cas system and/or at least one gRNA, wherein the at least one gRNA is encoded by DNA that encodes a crRNA and/or a tracrRNA.

Targeted genetic modifications can be generated by contacting a cell with a Cas protein and one or more guide RNAs that hybridize to one or more guide RNA recognition sequences within a target genomic locus. At least one of the one or more guide RNAs can form a complex with and can guide the Cas protein to at least one of the one or more guide RNA recognition sequences, and the Cas protein can cleave the target genomic locus within at least one of the one or more guide RNA recognition sequences. Cleavage by the Cas protein can create a double-strand break or a single-strand break (e.g., if the Cas protein is a nickase). The end sequences generated by the double-strand break or the single-strand break can then undergo recombination.

C. Methods for Introducing Oligonucleotides

Various methods and compositions are provided herein to allow for introduction of oligonucleotides into a cell. Methods for introducing oligonucleotides into various cell types are known and include, for example, stable transfection methods, transient transfection methods, and virus-mediated methods.

Transfection protocols as well as protocols for introducing oligonucleotides into cells may vary. Non-limiting transfection methods include chemical-based transfection methods using liposomes; nanoparticles; calcium phosphate (Graham et al. (1973) Virology 52 (2): 456-467, Bacchetti et al. (1977) Proc. Natl. Acad. Sci. U.S.A. 74 (4): 1590-1594, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, Sono-poration, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of oligonucleotides into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of oligonucleotides into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Methods for carrying out microinjection are well known. See, e.g., Nagy et al. (Nagy A, Gertsenstein M, Vintersten K, Behringer R., 2003, Manipulating the Mouse Embryo, Cold Spring Harbor, N.Y.: Cold Spring Harbor Laboratory Press); see also Meyer et al. (2010) Proc. Natl. Acad. Sci. USA 107:15022-15026 and Meyer et al. (2012) Proc. Natl. Acad. Sci. USA 109:9354-9359.

Other methods for introducing oligonucleotides into a cell can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, oligonucleotides can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule.

Introduction of oligonucleotides can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include 10¹², 10¹³, 10¹⁴, 10¹⁵, and 10¹⁶ vector genomes/mL.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

Introduction of oligonucleotides can also be accomplished by lipid nanoparticle (LNP)-mediated delivery. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more oligonucleotides for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyms, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

One common method for promoting uptake of reagents (e.g., antisense oligonucleotides) in cell culture involves use of cationic lipids to transfect nucleic acids. Mixing cationic lipid with negatively charged nucleic acids yields a complex that can cross cell membranes and release active nucleic acid into the cytoplasm of cells. It is also possible to electroporate reagents (e.g., antisense oligonucleotides) into cells. This method can be highly effective and useful for cell lines that cannot be readily transfected by lipid.

If the cells are in vivo (e.g., in an animal), administration to the animal can be by any suitable means. For example, administration can include parenteral routes of administration, such as intraperitoneal, intravenous, and subcutaneous. Parenteral administration means administration through injection or infusion. Parenteral administration includes, for example, subcutaneous administration, intravenous administration, intramuscular administration, intraarterial administration, intraperitoneal administration, or intracranial administration (e.g., intrathecal or intracerebroventricular administration).

In some methods, administration is by a means such that the reagent being introduced reaches neurons or the nervous system. This can be achieved, for example, by peripheral delivery or by direct delivery to the nervous system. See, e.g., Evers et al. (2015) Adv. Drug Deliv. Res. 87:90-103, herein incorporated by reference in its entirety for all purposes.

For reagents (e.g., antisense oligonucleotides) to reach the nervous system, they first have to cross the vascular barrier, made up of the blood brain barrier or the blood-spinal cord barrier. One mechanism that can be used to cross the vascular barrier is receptor-mediated endocytosis. Another mechanism that can be used is cell-penetrating peptide (CPP)-based delivery systems. Different CPPs use distinct cellular translocation pathways, which depend on cell types and cargos. For example, systemically delivered antisense oligonucleotides tagged with arginine-rich CPPs are able to cross the blood brain barrier. Another delivery mechanism that can be used is exosomes, which are extracellular vesicles known to mediate communication between cells through transfer of proteins and nucleic acids. For example, IV injection of exosomes transduced with short viral peptides derived from rabies virus glycoprotein (RVG) can result in crossing of the blood brain barrier and delivery to the brain.

Techniques are also available that bypass the vascular barriers through direct infusion into the cerebrospinal fluid. For example, reagents (e.g., antisense oligonucleotides) can be infused intracerebroventricularly (ICV), after which the reagents (e.g., antisense oligonucleotides) would have to pass the ependymal cell layer that lines the ventricular system to enter the parenchyma. Intrathecal (IT) delivery means delivery of the reagents (e.g., antisense oligonucleotides) into the subarachnoid space of the spinal cord. From here, reagents (e.g., antisense oligonucleotides) will have to pass the pia mater to enter the parenchyma. Reagents (e.g., antisense oligonucleotides) can be delivered ICT or IT through an outlet catheter that is connected to an implanted reservoir. Drugs can be injected into the reservoir and delivered directly to the CSF. Intranasal administration is an alternative route of delivery that can be used.

The scope of the present invention is defined by the claims appended hereto and is not limited by particular embodiments described herein; those skilled in the art, reading the present disclosure, will be aware of various modifications that may be equivalent to such described embodiments, or otherwise within the scope of the claims. In general, terminology is in accordance with its understood meaning in the art, unless clearly indicated otherwise. References cited within this specification, or relevant portions thereof, are incorporated herein by reference in their entireties.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an” in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

“Control” includes the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild type animal). In one experiment, a “test” (i.e., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

“Determining”, “measuring”, “evaluating”, “assessing”, “assaying” and “analyzing” includes any form of measurement and includes determining if an element is present or not. These terms include both quantitative and/or qualitative determinations. Assaying may be relative or absolute. “Assaying for the presence of” can be determining the amount of something present and/or determining whether or not it is present or absent.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure. Unless otherwise specified, any structural domain referred to herein refers to a TDP-43 structural domain.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous” refers to a location, nucleic acid or amino acid sequence that is found or occurs naturally within a cell or animal. For example, an endogenous TARDBP sequence of a non-human animal refers to a wildtype TARDBP sequence that naturally occurs at the endogenous TARDBP locus in the non-human animal.

The term “locus” refers to a specific location of a gene (or significant sequence), DNA sequence, polypeptide-encoding sequence, or position on a chromosome of the genome of an organism. For example, a “TARDBP locus” may refer to the specific location of a TARDBP gene, TARDBP DNA sequence, TARDBP 2-encoding sequence, or TARDBP position on a chromosome of the genome of an organism that has been identified as to where such a sequence resides. A “TARDBP locus” may comprise a regulatory element of a TARDBP gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). Other non-coding sequences of a gene include regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles, each at an endogenous locus of a homologous chromosome. Each pair of alleles represents the genotype of a specific genetic locus. Genotypes are described as homozygous if there are two identical alleles at a particular locus and as heterozygous if the two alleles differ.

“Operably linked” includes a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing, and can also include additional components whose presence is advantageous, for example, leader sequences and fusion partner sequences.

“Phenotype” includes a trait, or to a class or set of traits displayed by a cell or organism. In some embodiments, a particular phenotype may correlate with a particular allele or genotype. In some embodiments, a phenotype may be discrete; in some embodiments, a phenotype may be continuous. A phenotype may comprise viability or cellular fitness of a cell. A phenotype may comprise the expression levels, cellular localization and/or solubility/stability profile of a protein, e.g., a mutant TDP-43 polypeptide, each of which phenotypes may be determined using well-known methods such as Western Blot analysis, fluorescent in situ hybridization, qualitative RT-PCR, etc.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Reference” includes a standard or control agent, cell, animal, cohort, individual, population, sample, sequence or value against which an agent, cell, animal, cohort, individual, population, sample, sequence or value of interest is compared. In some embodiments, a reference agent, cell, animal, cohort, individual, population, sample, sequence or value is tested and/or determined substantially simultaneously with the testing or determination of the agent, cell, animal, cohort, individual, population, sample, sequence or value of interest. In some embodiments, a reference agent, cell, animal, cohort, individual, population, sample, sequence or value is a historical reference, optionally embodied in a tangible medium. In some embodiments, a reference may refer to a control. A “reference” also includes a “reference cell”. A “reference cell” may have a modification as described herein, a modification that is different as described herein or no modification (i.e., a wild type cell). Typically, as would be understood by those skilled in the art, a reference agent, cell, animal, cohort, individual, population, sample, sequence or value is determined or characterized under conditions comparable to those utilized to determine or characterize the agent, animal (e.g., a mammal), cohort, individual, population, sample, sequence or value of interest.

The term “variant” refers to a nucleotide sequence that differs from a reference nucleotide sequence (e.g., by one nucleotide) or a protein sequence that differs from a reference amino acid sequence (e.g., by one amino acid), but that retain the biological function of the reference sequence. In some embodiments, variants differ from the reference sequence due to degeneracy of the genetic code and/or a conservative codon/amino acid substitution.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized in Table 1 below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

Non-limiting exemplary embodiments include the following.

### Embodiment 1

A non-human animal cell comprising a mutated TARDBP gene that encodes a mutant TDP-43 polypeptide,
wherein the mutant TDP-43 polypeptide lacks a functional structural domain found in a wildtype TDP-43 polypeptide, and
wherein the non-human animal or non-human animal cell expresses the mutant TDP-43 polypeptide,
optionally wherein the wildtype TDP-43 polypeptide comprises a sequence set forth as SEQ ID NO:1, SEQ ID NO:3, or SEQ ID NO:5.

### Embodiment 2

The non-human animal cell of embodiment 1, wherein the mutant TDP-43 polypeptides lacks a functional structural domain comprising the nuclear localization signal (NLS), the RNA recognition motif 1 (RRM1), the RNA recognition motif 2 (RRM2), the putative nuclear export signal (E), the prion like domain (PLD), or a combination thereof.

### Embodiment 3

The non-human animal cell of embodiment 1 or embodiment 2, wherein the non-human animal cell is an embryonic stem (ES) cell, an embryoid body, or an embryonic stem cell derived motor neuron (ESMN).

### Embodiment 4

The non-human animal cell of any one of the preceding embodiments, wherein the mutated TARDBP gene is a mutated TARDBP gene of the non-human animal.

### Embodiment 5

The non-human animal cell of any one of embodiments 1-3, wherein the mutated TARDBP gene is a mutated human TARDBP gene.

### Embodiment 6

The non-human animal cell of any one of the preceding embodiments, wherein the mutant TDP-43 polypeptide lacks a functional structural domain due to one or more of the following:
(a) a point mutation of an amino acid in the NLS,
(b) a point mutation of an amino acid in the RRM1,
(c) a point mutation of an amino acid in the RRM2,
(d) a deletion of at least a portion of the nuclear export signal, and
(e) a deletion of at least a portion of the prion-like domain.

### Embodiment 7

The non-human animal cell of embodiment 6, wherein
(a) the point mutation of an amino acid in the NLS comprises K82A K83A, R84A, K95A, K97A, K98A, or a combination thereof,
(b) the point mutation in RRM1 comprises F147L and/or F149L,
(c) the point mutation in RRM2 comprises F194L and/or F229L,
(d) the deletion of at least a portion of the nuclear export signal deletion comprises a deletion of the amino acids at and between positions 239 and 250 of a wildtype TDP-43 polypeptide, and
(e) the deletion of at least a portion of the prion-like domain comprises a deletion of the amino acids at and between positions 274 and 414 of a wildtype TDP-43 polypeptide.

### Embodiment 8

The non-human animal cell of any one of the preceding embodiments, wherein the mutant TDP-43 polypeptide comprises K82A K83A, R84A, K95A, K97A, and K98A.

### Embodiment 9

The non-human animal cell of any one of the preceding embodiments, wherein the mutant TDP-43 polypeptide lacks the prion-like domain between and including the amino acids at positions 274 to 414 of a wildtype polypeptide.

### Embodiment 10

The non-human animal cell of any one of the preceding embodiments, wherein the mutant TDP-43 polypeptide comprises F147L and F149L.

### Embodiment 11

The non-human animal cell of any one of the preceding embodiments, wherein the mutant TDP-43 polypeptide comprises F194L and F229L.

### Embodiment 12

The non-human animal cell of any one of the preceding embodiments, wherein the mutant TDP-43 polypeptide lacks the nuclear export signal between and including the amino acids at positions 239 and 250.

### Embodiment 13

The non-human animal cell of any one of the preceding embodiments, wherein the mutated TARDBP gene that encodes a mutant TDP-43 polypeptide replaces an endogenous TARDBP gene at an endogenous TARDBP locus.

### Embodiment 14

The non-human animal cell of embodiment 13, wherein the non-human animal cell is heterozygous for the mutated TARDBP gene that encodes a mutant TDP-43 polypeptide.

### Embodiment 15

The non-human animal cell of embodiment 13, wherein the non-human animal cell is homozygous for the mutated TARDBP gene that encodes a mutant TDP-43 polypeptide.

### Embodiment 16

The non-human animal cell of any one of embodiments 1-14, wherein the non-human animal cell further comprises a TARDBP gene comprising a knockout mutation.

### Embodiment 17

The non-human animal cell of embodiment 16, wherein the knockout mutation comprises a conditional knockout mutation.

### Embodiment 18

The non-human animal cell of embodiment 16 or embodiment 17, wherein the knockout mutation comprises a site-specific recombination recognition sequence.

### Embodiment 19

The non-human animal cell of any one of embodiments 16-18, wherein the knockout mutation comprises a loxp sequence.

### Embodiment 20

The non-human animal cell of embodiment 19, wherein the loxp sequence flanks exon 3 of the TARDBP gene comprising a knockout mutation.

### Embodiment 21

The non-human animal cell of embodiment 16, wherein the knockout mutation comprises a deletion of the entire coding sequence of TDP-43 peptide.

### Embodiment 22

The non-human animal cell of any one of embodiments 16-21, wherein the non-human animal cell is heterozygous for the modified TARDBP locus and comprises
(i) at one chromosome at an endogenous TARDBP locus, a replacement of an endogenous TARDBP gene with the mutated TARDBP gene that encodes a mutant TDP-43 polypeptide, and
(ii) at the other homologous chromosome at the endogenous TARDBP locus, either the TARDBP gene comprising the knockout mutation or a wildtype TARDBP gene.

### Embodiment 23

The non-human animal cell of any one of the preceding embodiments, wherein the non-human animal cell does not express a wildtype TDP-43 polypeptide.

### Embodiment 24

The non-human animal cell of any one of embodiments 1-22, wherein the non-human animal cell expresses a wildtype TDP-43 polypeptide.

### Embodiment 25

The non-human animal cell of any one of the preceding embodiments, comprising:
(i) mRNA transcript levels of the mutated TARDBP gene that comparable to mRNA transcript levels of a wildtype TARDBP gene in a control cell,
(ii) increased levels of the mutant TDP-43 polypeptide compared to levels of wildtype TDP-43 polypeptide in a control cell,
(iii) a higher concentration of mutant TDP-43 polypeptide found in the cytoplasm than in the nucleus, e.g., of a motor neuron,
(iv) mutant TDP-43 polypeptide with increased insolubility compared to a wildtype TDP-43 polypeptide
(v) cytoplasmic aggregates comprising the mutant TDP-43 polypeptide,
(vi) increased splicing of cryptic exons, and/or
(vii) decreased levels of the alternatively spliced TDP-43 form.

### Embodiment 26

A non-human animal cell comprising (i) at one chromosome at an endogenous TARDBP locus, a conditional knockout mutation of the TARDBP gene, and (ii) at the other homologous chromosome at the endogenous TARDBP locus, a deletion of the entire TARDBP coding sequence.

### Embodiment 27

The non-human animal cell of any one of the preceding embodiments, wherein the cell is an embryonic stem (ES) cell, a primitive ectoderm cell, or a motor neuron derived from a motor neuron (ESMN).

### Embodiment 28

The non-human animal cell of any one of the preceding embodiments, wherein the non-human animal cell is a rodent cell.

### Embodiment 29

The non-human animal cell of any one of the preceding embodiments, wherein the non-human animal cell is a rat cell.

### Embodiment 30

The non-human animal cell of any one of embodiments 1-28, wherein the non-human animal cell is a mouse cell.

### Embodiment 31

The non-human animal cell of any one of the preceding embodiments, wherein the non-human animal cell is cultured in vitro.

### Embodiment 32

A non-human animal tissue comprising the non-human animal cell of any one of the preceding embodiments.

### Embodiment 33

A composition comprising the non-human animal cell or tissue of any one of the preceding embodiments.

### Embodiment 34

A method of making a non-human animal or a non-human animal cell that expresses a mutant TDP-43 polypeptide comprising modifying the genome of the non-human animal or non-human animal cell to comprise a mutated TARDBP gene that encodes the mutant TDP-43 polypeptide, wherein the mutant TDP-43 polypeptide lacks a functional structural domain compared to a wildtype TDP-43, optionally wherein the wildtype TDP-43 polypeptide comprises a sequence set forth as SEQ ID NO:1, SEQ ID NO:3, or SEQ ID NO:5.

### Embodiment 35

The method of embodiment 34, wherein modifying comprises replacing an endogenous TARDBP gene with the mutated TARDBP gene that encodes the mutant TDP-43 polypeptide.

### Embodiment 36

The method of embodiment 34 or embodiment 35, wherein modifying further comprises replacing an endogenous TARDBP gene with a TARDBP gene comprising a knockout mutation.

### Embodiment 37

The method of embodiment 36, wherein the knockout mutation comprises a conditional knockout mutation.

### Embodiment 38

The method of embodiment 37, further comprising culturing the cell in conditions that eliminates expression of the TARDBP gene comprising a knockout mutation.

### Embodiment 39

A method of identifying a therapeutic candidate for the treatment of a disease, the method comprising
(a) contacting non-human animal cell or tissue of any one of embodiments 1-31 or the composition of embodiment 32 with the candidate agent,
(b) evaluating the phenotype and/or TDP-43 biological activity of the non-human cell or tissue, and
(c) identifying the candidate agent that restores to the non-human cell or tissue a phenotype and/or TDP-43 biological activity comparable to that of a control cell or tissue that expresses a wildtype TDP-43 polypeptide.

### Embodiment 40

A method of evaluating the biological function of a TDP-43 structural domain comprising
(a) modifying an embryonic stem (ES) cell to comprise a mutated TARDBP gene that encodes a mutant TDP-43 polypeptide that lacks a functional structural domain selected from the group consisting of the nuclear localization signal (NLS), the first RNA recognition motif (RRM1), the first RNA recognition motif (RRM2), the putative nuclear export signal (E), the prion like domain (PLD), and a combination thereof,
(b) optionally differentiating the modified ES cell in vitro and/or obtaining a genetically modified non-human animal from the modified ES cell, and
(c) evaluating the phenotype and/or TDP-43 biological activity of the genetically modified ES cell, primitive ectoderm derived therefrom, motor neurons derived therefrom, or a non-human animal derived therefrom.

### Embodiment 41

The method of embodiment 39 or embodiment 40, wherein the phenotype is evaluated by cell culture, fluorescence in situ hybridization, Western Blot analysis, or a combination thereof.

### Embodiment 42

The method of any one of embodiments 39-41, wherein evaluating the phenotype comprises measuring the viability the genetically modified ES cell, primitive ectoderm derived therefrom, motor neurons derived therefrom, or a non-human animal derived therefrom.

### Embodiment 43

The method of any one of embodiments 39-42, wherein the evaluating the phenotype comprises determining the cellular location of the mutant TDP-43 polypeptide.

### Embodiment 44

The method of any one of embodiments 39-43, wherein evaluating the biological activity of the mutant TDP-43 polypeptide comprises measuring the splice products of genes comprising cryptic exons regulated by TDP-43.

### Embodiment 45

The method of embodiment 44, wherein the gene comprising cryptic exons regulated by TDP-43 comprises Crem, Fyxd2, Clf1.

### Embodiment 46

The method of any one of embodiments 39-45, wherein evaluating the biological activity of the mutant TDP-43 polypeptide comprises measuring the levels of an alternatively spliced TDP-43.

### Embodiment 47

An antisense oligonucleotide comprising a gapmer motif targeting a TDP-43 mRNA sequence that encodes a PLD of a TDP-43 polypeptide and/or comprises untranslated sequences downstream of exon 6 and upstream of exon 7,
optionally wherein the TDP-mRNA comprises a sequence between an alternative 5′ splice site within exon 6 and a downstream alternative 3′ splice site,
optionally wherein the alternative 5′ splice site correlates to a TARDBP genomic position selected from the group consisting of (a) mouse chromosome 4:148,618,647; (b) mouse chromosome 4:148,618,665; (c) mouse chromosome 4:148,618,674, and (d) any corresponding position in a human TARDBP gene and/or wherein the alternative 3′ splice junction correlates to a TARDBP genomic position of chromosome 4: 148,617,705.

### Embodiment 48

An siRNA comprising a sequence targeting a TDP-43 mRNA sequence that encodes a PLD of a TDP-43 polypeptide and/or comprises untranslated sequences downstream of exon 6 and upstream of exon 7,
optionally wherein the TDP-mRNA sequence is between an alternative 5′ splice site within exon 6 and a downstream alternative 3′ splice site,
optionally wherein the alternative 5′ splice site correlates to a TARDBP genomic position selected from the group consisting of (a) mouse chromosome 4:148,618,647; (b) mouse chromosome 4:148,618,665; (c) mouse chromosome 4:148,618,674, and (d) any corresponding position in a human TARDBP gene and/or wherein the alternative 3′ splice junction correlates to a TARDBP genomic position of chromosome 4: 148,617,705.

### Embodiment 49

A CRISPR/Cas system comprising a Cas9 protein and at least one gRNA, wherein the gRNA recognizes a sequence at or near sequences encoding for alternative splice sites that result in alternative mRNA that encode a truncated TDP-43 polypeptide lacking a PLD,
optionally wherein the alternative splice sites comprises an alternative 5′ splice site within exon 6 and a downstream alternative 3′ splice site,
optionally wherein the alternative 5′ splice site correlates to a TARDBP genomic position selected from the group consisting of (a) mouse chromosome 4:148,618,647; (b) mouse chromosome 4:148,618,665; (c) mouse chromosome 4:148,618,674, and (d) any corresponding position in a human TARDBP gene and/or wherein the alternative 3′ splice junction correlates to a TARDBP genomic position of chromosome 4: 148,617,705.

### Embodiment 50

A non-human animal comprising the embryonic stem cell of embodiment 2.

### Embodiment 51

A non-human animal comprising a mutated TARDBP gene that encodes a mutant TDP-43 polypeptide,
wherein the mutant TDP-43 polypeptide lacks a functional structural domain compared to a wildtype TDP-43 polypeptide, and
wherein the non-human animal expresses the mutant TDP-43 polypeptide, optionally wherein the wildtype TDP-43 polypeptide comprises a sequence set forth as SEQ ID NO:1, SEQ ID NO:3, or SEQ ID NO:5.

### Embodiment 52

The non-human animal of embodiment 51, wherein the mutant TDP-43 polypeptides lacks a functional structural domain comprising the nuclear localization signal (NLS), the RNA recognition motif 1 (RRM1), the RNA recognition motif 2 (RRM2), the putative nuclear export signal (E), the prion like domain (PLD), or a combination thereof.

### Embodiment 53

The non-human animal of embodiment 51 or embodiment 52, wherein the mutated TARDBP gene is a mutated TARDBP gene of the non-human animal.

### Embodiment 54

The non-human animal of any one of embodiments 51-53, wherein the mutated TARDBP gene is a mutated human TARDBP gene.

### Embodiment 55

The non-human animal of any one of embodiments 51-54, wherein the mutant TDP-43 polypeptide lacks a functional structural domain due to one or more of the following:
(a) a point mutation of an amino acid in the NLS,
(b) a point mutation of an amino acid in the RRM1,
(c) a point mutation of an amino acid in the RRM2,
(d) a deletion of at least a portion of the nuclear export signal, and
(e) a deletion of at least a portion of the prion-like domain.

### Embodiment 56

The non-human animal of embodiment 55, wherein
(a) the point mutation of an amino acid in the NLS comprises K82A K83A, R84A, K95A, K97A, K98A, or a combination thereof,
(b) the point mutation in RRM1 comprises F147L and/or F149L,
(c) the point mutation in RRM2 comprises F194L and/or F229L,
(d) the deletion of at least a portion of the nuclear export signal deletion comprises a deletion of the amino acids at and between positions 239 and 250 of a wildtype TDP-43 polypeptide, and
(e) the deletion of at least a portion of the prion-like domain comprises a deletion of the amino acids at and between positions 274 and 414 of a wildtype TDP-43 polypeptide.

### Embodiment 57

The non-human animal of any one of embodiments 51-56, wherein the mutant TDP-43 polypeptide comprises K82A K83A, R84A, K95A, K97A, and K98A.

### Embodiment 58

The non-human animal of any one of embodiments 51-57, wherein the mutant TDP-43 polypeptide lacks the prion-like domain between and including the amino acids at positions 274 to 414 of a wildtype polypeptide.

### Embodiment 59

The non-human animal of any one of embodiments 51-58, wherein the mutant TDP-43 polypeptide comprises F147L and F149L.

### Embodiment 60

The non-human animal of any one of embodiments 51-59, wherein the mutant TDP-43 polypeptide comprises F194L and F229L.

### Embodiment 61

The non-human animal of any one of embodiments 51-60, wherein the mutant TDP-43 polypeptide lacks the nuclear export signal between and including the amino acids at positions 239 and 250.

### Embodiment 62

The non-human animal of any one of embodiments 51-61, wherein the mutated TARDBP gene that encodes a mutant TDP-43 polypeptide replaces an endogenous TARDBP gene at an endogenous TARDBP locus.

### Embodiment 63

The non-human animal of embodiment 62, wherein the non-human animal is heterozygous for the mutated TARDBP gene that encodes a mutant TDP-43 polypeptide.

### Embodiment 64

The non-human animal of any one of embodiments 51-63, wherein the non-human animal further comprises a TARDBP gene comprising a knockout mutation.

### Embodiment 65

The non-human animal of embodiment 64, wherein the knockout mutation comprises a conditional knockout mutation.

### Embodiment 66

The non-human animal embodiment 64 or embodiment 65, wherein the knockout mutation comprises a site-specific recombination recognition sequence.

### Embodiment 67

The non-human animal of any one of embodiments 64-66, wherein the knockout mutation comprises a loxp sequence.

### Embodiment 68

The non-human animal of embodiment 67, wherein the loxp sequence flanks exon 3 of the TARDBP gene comprising a knockout mutation.

### Embodiment 69

The non-human animal of embodiment 64, wherein the knockout mutation comprises a deletion of the entire coding sequence of TDP-43 peptide.

### Embodiment 70

The non-human animal of any one of embodiments 64-69, wherein the non-human animal is heterozygous for the modified TARDBP locus and comprises
(i) at one chromosome at an endogenous TARDBP locus, a replacement of an endogenous TARDBP gene with the mutated TARDBP gene that encodes a mutant TDP-43 polypeptide, and
(ii) at the other homologous chromosome at the endogenous TARDBP locus, either the TARDBP gene comprising the knockout mutation or a wildtype TARDBP gene.

### Embodiment 71

The non-human animal of any one of embodiments 50-70, wherein the non-human animal expresses a wildtype TDP-43 polypeptide.

### Embodiment 72

The non-human animal of any one of embodiments 50-71, comprising:
(i) mRNA transcript levels of the mutated TARDBP gene that comparable to mRNA transcript levels of a wildtype TARDBP gene in a control animal,
(ii) increased levels of the mutant TDP-43 polypeptide compared to levels of wildtype TDP-43 polypeptide in a control animal,
(iii) a higher concentration of mutant TDP-43 polypeptide found in the cytoplasm than in the nucleus, e.g., of a motor neuron,
(iv) mutant TDP-43 polypeptide with increased insolubility compared to a wildtype TDP-43 polypeptide
(v) cytoplasmic aggregates comprising the mutant TDP-43 polypeptide,
(vi) increased splicing of cryptic exons,
(vii) decreased levels of the alternatively spliced TDP-43 form,
(viii) denervation of muscle tissue comprised of predominantly fast twitch muscles, such as anterior tibialis muscles and/or
(ix) normal innervation of muscle tissues comprised of predominantly low twitch muscles, such as intercostal muscles.

### Embodiment 73

A non-human animal comprising at an endogenous TARDBP locus a TARDBP gene comprising a conditional knockout mutation and at an other endogenous TARDBP locus of a homologous chromosome a TARDBP gene comprising a deletion of the entire TARDBP coding sequence.

### Embodiment 74

The non-human animal of any one of embodiments 50-73, wherein the non-human animal is a rodent.

### Embodiment 75

The non-human animal of any one of embodiments 50-74, wherein the non-human animal a rat.

### Embodiment 76

The non-human animal of any one of embodiments 50-74, wherein the non-human animal is a mouse.

### Embodiment 77

A method of identifying a therapeutic candidate for the treatment of a disease, the method comprising
(a) contacting the non-human animal any one of embodiments 50-76 with the candidate agent,
(b) evaluating the phenotype and/or TDP-43 biological activity of the non-human animal, and
(c) identifying the candidate agent that restores to the non-human a phenotype and/or TDP-43 biological activity.

### Embodiment 78

A mutant TDP-43 polypeptide comprising a sequence set forth as SEQ ID NO:1, 3, or 5 modified to comprise to one or more of the following:
(a) a point mutation of an amino acid in the NLS,
(b) a point mutation of an amino acid in the RRM1,
(c) a point mutation of an amino acid in the RRM2,
(d) a deletion of at least a portion of the nuclear export signal, and
(e) a deletion of at least a portion of the prion-like domain.

### Embodiment 79

The mutant TDP-43 polypeptide of embodiment 78, wherein
(a) the point mutation of an amino acid in the NLS comprises K82A K83A, R84A, K95A, K97A, K98A, or a combination thereof,
(b) the point mutation in RRM1 comprises F147L and/or F149L,
(c) the point mutation in RRM2 comprises F194L and/or F229L,
(d) the deletion of at least a portion of the nuclear export signal deletion comprises a deletion of the amino acids at and between positions 239 and 250 of a wildtype TDP-43 polypeptide, and
(e) the deletion of at least a portion of the prion-like domain comprises a deletion of the amino acids at and between positions 274 and 414 of a wildtype TDP-43 polypeptide.

### Embodiment 80

The mutant TDP-43 polypeptide of embodiment 78 or embodiment 79 comprising a K82A mutation, a K83A mutation, a R84A mutation, a K95A mutation, a K97A mutation, and/or a K98A mutation.

### Embodiment 81

The mutant TDP-43 polypeptide of any one of embodiments 78-80, comprising a deletion of the prion-like domain between and including the amino acids at positions 274 to 414 of a wildtype polypeptide.

### Embodiment 82

The mutant TDP-43 polypeptide of any one of embodiments 78-81, wherein the mutant TDP-43 polypeptide comprises a F147L mutation and/or a F149L mutation.

### Embodiment 83

The mutant TDP-43 polypeptide of any one of embodiments 78-82, wherein the mutant TDP-43 polypeptide comprises a F194L mutation and/or a F229L mutation.

### Embodiment 84

The mutant TDP-43 polypeptide of any one of embodiments 78-83, wherein the mutant TDP-43 polypeptide lacks the nuclear export signal between and including the amino acids at positions 239 and 250.

### Embodiment 85

A nucleic acid comprising a nucleic acid sequence encoding the mutant TDP-43 polypeptide of any one of embodiments 78-84.

### Embodiment 86

The nucleic acid of embodiment 85, further comprising from 5′ to 3′: a 5′ homology arm, the nucleic acid sequence encoding the mutant TDP-43 polypeptide, and a 3′ homology arm, wherein the nucleic acid undergoes homologous recombination in a rodent cell.

### Embodiment 87

The nucleic acid of embodiment 86, wherein the 5′ and 3′ homology arms are homologous to rat sequences such that the nucleic acid undergoes homologous recombination at an endogenous rat TARDBP locus and the nucleic acid sequence encoding the mutant TDP-43 polypeptide replaces the endogenous TARDBP coding sequence.

### Embodiment 88

The nucleic acid of embodiment 86, wherein the 5′ and 3′ homology arms are homologous to mouse sequences such that the nucleic acid undergoes homologous recombination at an endogenous mouse TARDBP locus and the nucleic acid sequence encoding the mutant TDP-43 polypeptide replaces the endogenous TARDBP coding sequence.

### Embodiment 89

A method of selectively decreasing TDP-43 mRNA that encode a TDP-43 polypeptide comprising a PLD while sparing alternative TDP-43 mRNA that encode a truncated TDP-43 lacking a PLD in a cell, the method comprising introducing into the cell:
(i) an antisense oligonucleotide comprising a gapmer motif targeting a TDP-43 mRNA sequence that encodes a PLD of a TDP-43 polypeptide and/or comprises untranslated sequences downstream of exon 6 and upstream of exon 7,
(ii) an siRNA comprising a sequence targeting a TDP-43 mRNA sequence that encodes a PLD of a TDP-43 polypeptide and/or comprises untranslated sequences downstream of exon 6 and upstream of exon 7, and/or
(iii) a CRISPR/Cas system comprising a Cas9 protein and at least one gRNA, wherein the gRNA recognizes a sequence at or near sequences encoding for alternative splice sites that result in alternative mRNA that encode a truncated TDP-43 polypeptide lacking a PLD.

### Embodiment 90

The method of embodiment 89, wherein:
(i) the antisense oligonucleotide is the ASO of embodiment 47,
(ii) the siRNA is the siRNA of embodiment 48, and/or
(iii) the CRISPR/Cas system is the CRISPR/Cas system of embodiment 49.

### Embodiment 91

The method of embodiment 89 or embodiment 90, wherein the cell is in vivo.

BRIEF DESCRIPTION OF SEQUENCES

| SEQ | |
|---|---|
| ID NO | DESCRIPTION |
| 1 | NP_663531-Wildtype mouse TDP-43 (Protein) |
| 2 | NM_145556.4-Wildtype mouse TARDBP |
| | coding sequence (DNA) |
| 3 | NP_001011979-Wildtype rat TDP-43 (Protein) |
| 4 | NM_001011979.2-Wildtype rat TARDBP |
| | coding sequence (DNA) |
| 5 | NP_031401.1-Wildtype human TDP-43 (Protein) |
| 6 | NM_007375.3-Wildtype human TARDBP |
| | coding sequence (DNA) |
| 7 | RRM1 RNP2 consensus sequence (Protein) |
| 8 | RRM1 RNP1 consensus sequence (Protein) |
| 9 | RRM2 RNP2 consensus sequence (Protein) |
| 10 | RRM2 RNP1 consensus sequence (Protein) |
| 11 | TDP-43 Ex3-Ex4 assay Forward Primer (DNA) |
| 12 | TDP-43 Ex3-Ex4 assay Reverse Primer (DNA) |
| 13 | TDP-43 Ex3-Ex4 Probe (DNA) |
| 14 | Crem Ex1-Ex2 assay Forward Primer (DNA) |
| 15 | Crem Ex1-Ex2 assay Reverse Primer (DNA) |
| 16 | Crem Ex1-Ex2 Probe (DNA) |
| 17 | Crem Ex1-Cryptic assay Forward Primer (DNA) |
| 18 | Crem Ex1-Cryptic assay Reverse Primer (DNA) |
| 19 | Crem Ex1-Cryptic Probe (DNA) |
| 20 | Crem Cryptic-Ex2 assay Forward Primer (DNA) |
| 21 | Crem Cryptic-Ex2 assay Reverse Primer (DNA) |
| 22 | Crem Cryptic-Ex2 Probe (DNA) |
| 23 | Fyxd2Ex3-Ex4 assay Forward Primer (DNA) |
| 24 | Fyxed Ex3-Ex4 assay Reverse Primer (DNA) |
| 25 | Fyxed Ex3-Ex4 Probe (DNA) |
| 26 | Fyxed Ex3-Cryptic assay Forward Primer (DNA) |
| 27 | Fyxed Ex3-Cryptic assay Reverse Primer (DNA) |
| 28 | Fyxed Ex3-Cryptic Probe (DNA) |
| 29 | Fyxed Cryptic-Ex4 assay Forward Primer (DNA) |
| 30 | Fyxed Cryptic-Ex4assay Reverse Primer (DNA) |
| 31 | Fyxed Cryptic-Ex4 Probe (DNA) |
| 32 | Crlf1 Ex1-Ex2 assay Forward Primer (DNA) |
| 33 | Crlf1 Ex1-Ex2 assay Reverse Primer (DNA) |
| 34 | Crlf1 Ex1-Ex2 Probe (DNA) |
| 35 | Crlf1 Ex1-Cryptic assay Forward Primer (DNA) |
| 36 | Crlf1 Ex1-Cryptic assay Reverse Primer (DNA) |
| 37 | Crlf1 Ex1-Cryptic Probe (DNA) |
| 38 | Crlf1 Cryptic-Ex2 assay Forward Primer (DNA) |
| 39 | Crlf1 Cryptic-Ex2 assay Reverse Primer (DNA) |
| 40 | Crlf1 Cryptic-Ex2 Probe (DNA) |
| 41 | TDP-43 Ex6-Ex7 assay Forward Primer (DNA) |
| 42 | TDP-43 Ex6-Ex7 assay Reverse Primer (DNA) |
| 43 | TDP-43 Ex6-Ex7 Probe (DNA) |

### EXAMPLES

The following examples are provided for illustrative purposes only and are not intended to limit the scope of the invention.

### Example 1: Generation of Embryonic Stem Cells Expressing a Mutated TARDBP Gene

Since TDP-43 is essential for viability, embryonic stem (ES) cells comprising a conditional knockout on a first endogenous TDP-43 allele and a mutation on the other second endogenous TDP-43 allele may be generated such that wildtype TDP-43 from the first endogenous allele sustains viability of the ES cell until activation of the condition, after which activation the effects of the mutant TDP-43 polypeptide expressed from the second allele may be ascertained.

To evaluate the biological, biochemical, and/or pathogenic role(s) played by various TDP-43 structural domains, mouse ES cells were modified to comprise: (i) at an endogenous TARDBP locus, a conditional knockout mutation, and (ii) at the other TARDBP locus on a homologous chromosome, a mutated TARDBP gene that encodes a mutant TDP-43 polypeptide in which one of the five structural domains—the nuclear localization signal (NLS), RNA recognition motif 1 (RRM1), RNA recognition motif 2 (RRM2), a putative export signal (E), or the prion like domain (PLD)—was either altered in ways predicted to abolish their functions or deleted. See, FIG. 3. Both (1) the phenotype and (2) the biological activity of mutant TDP-43 polypeptide of cells harboring the mutated TARDBP gene(s) and expressing a mutant TDP-43 polypeptide lacking a functional NLS, RRM1, RRM2, E, or PLD were analyzed as respectively described in Examples 2 and 3.

The conditional allele was designed based on previously published work that shows deletion of TDP-43 exon 3 produces no functional protein. Chiang et al. (2010) Proc Natl Acad Sci USA 107:16320-324. Exon 3 of the endogenous mouse TARDBP gene was foxed with loxp sites. After cre-mediated recombination, deletion of the genomic coordinates chr4:147995844-147996841 was effected. ES cells comprising the floxed exon 3 were further modified with a mutated TARDBP gene as described herein. As a control, mouse ES cells modified with the conditional knockout mutation on one allele and a deletion from the start codon of the second exon to the stop codon (genomic coordinates chr4:147992370-147999471) on the other allele were also created.

### Example 2: Phenotypic Analyses of Cells Expressing a Mutated TARDBP Gene

The phenotype of the embryonic stem (ES) cells generated in Example 1, primitive ectoderm derived therefrom, or motor neurons derived therefrom (ESMNs) was analyzed by evaluating the viability of the cells and the localization and stability of the mutant TDP-43 polypeptides.

Notably, ES cells expressing a mutant TDP-43 polypeptide lacking a functional NLS or functional PLD were viable; although, cells expressing the mutant TDP-43 polypeptide lacking a functional PLD appeared to have reduced fitness. FIG. 4. Neither ES cells nor ESMNs expressing a mutant TDP-43 polypeptide lacking a functional RRM1 or RRM2 remained viable. FIGS. 4 and 5.

A mutant TDP-43 polypeptide lacking a functional NLS redistributed from the nucleus to the cytoplasm in ESMNs, and the mutant TDP-43 accumulated in many large aggregate-like inclusions reminiscent of ALS pathology. FIGS. 6-8. Lack of a functional NLS caused extensive cytoplasmic aggregation of the mutant TDP-43 polypeptide, with loss of nuclear staining. FIGS. 7-8. Mutant TDP-43 polypeptides lacking a functional PLD also redistributed to the cytoplasm of ESMNs and accumulated in punctate inclusions that appeared to be less abundant and qualitatively different than those produced by the mutant TDP-43 polypeptide lacking a functional NLS. FIGS. 6-8. Deletion of the PLD caused the greatest degree of mislocalization of the mutant TDP-43 polypeptide to the cytoplasm, although nuclear staining was retained. FIGS. 7-8.

Mutant TDP-43 polypeptides lacking a functional NLS or PLD exhibited increased solubility of the mutant TDP-44. FIG. 9A. The solubility of mutant TDP-43 polypeptides lacking a functional E or RRM1 was unchanged compared wildtype TDP-43 polypeptides. FIG. 9A. Although there was no difference in mRNA expression levels for any of the mutant TDP-43 polypeptides, an increase in protein levels was seen for mutant TDP-43 polypeptides lacking a functional NLS, PLD or RRM1. FIG. 9B. Since the mRNA expression levels for these mutant TDP-43 polypeptides were comparable to expression levels of wildtype TDP-43, the increased protein levels were likely due to the increased stability of the mutant TDP-43 polypeptides. FIG. 9C.

The materials and methods used to analyze the phenotype of cells expressing a mutant TDP-43 polypeptide lacking a functional structural domain are described below.

Cell Culture

The ability of a mutant TDP-43 protein, as the only form of the protein expressed by the cell, to support viability of embryonic stem (ES) cells and motor neurons derived from them (ESMNs) was tested by differentiation in culture. ES cells were cultured in embryonic stem cell medium (ESM; DMEM+15% fetal bovine serum+penicillin/streptomycin+glutamine+non-essential amino acids+nucleosides+β-mercaptoethanol+sodium pyruvate+LIF) for 2 days, during which the medium was changed daily. ES medium was replaced with 7 mL of ADFNK medium (advanced DMEM/F12+neurobasal medium+10% knockout serum+penicillin/streptomycin+glutamine+β-mercaptoethanol) 1 hour before trypsinization. ADFNK medium was aspirated, and ESCs were trypsinized with 0.05% trypsin-EDTA. Pelleted cells were resuspended in 12 mL of ADFNK and grown for two days in suspension. Cells were cultured for a further 4 days in ADFNK supplemented with retinoic acid (RA), smoothened agonist and purmorphamine to obtain limb-like motor neurons (ESMNs). Dissociated motor neurons were plated and matured in embryonic-stem-cell-derived motor neuron medium (ESMN; neurobasal medium+2% horse serum+B27+glutamine+penicillin/streptomycin+β-mercaptoethanol+10 ng/mL GDNF, BDNF, CNTF). The conditional knockout allele was activated using cre recombinase delivered via electroporation at the ES cell stage (FIGS. 4, 6-9) or seven days after plating (FIG. 5).

Intracellular Localization of Mutant TDP-43 Polypeptides

The intracellular localization of TDP-43 mutants was analyzed using an antibody that recognizes the N-terminus of the TDP-43 polypeptide (α-TDP-43 N-term) and an antibody that recognizes the C-terminal prion like domain of the TDP-43 polypeptide (α-TDP-43 C-term) (Proteintech, Rosemont, Ill.). Soluble cytoplasmic protein extracts were prepared by incubating ES cell-derived MNs in ice-cold lysis buffer (10 mM KCl, 10 mM Tris-HCl, pH 7.4, 1 mM MgCl2, 1 mM DTT, 0.01% NP-40) supplemented with protease and phosphatase inhibitors (Roche) for 10 minutes on ice. Cells were then passed through a 27-gauge syringe five times. Following centrifugation at 4° C. for 5 minutes at 4000 rpm, the protein supernatant that comprises the soluble cytoplasmic extract was collected. Insoluble nuclear protein extracts were prepared by resuspending the pellet in an equal volume of RBS-100 buffer (10 mMTris-HCl pH 7.4, 2.5 mM MgCl2, 100 mM NaCl, 0.1% NP-40) supplemented with protease and phosphatase. Equal volumes of 2×SDS sample buffer was added to each fraction and samples were heated to 90° C. Equal volumes of each fraction were then loaded onto a 14% SDS gel and electrophoresed for 50 min at 225V followed by western blotting for TDP-43 using the α-TDP-43-N-term antibody or the α-TDP-43-C-term antibody, the latter of which would not recognize the PLD deletion mutants. Densitometry was performed using ImageJ. FIG. 6B. The ratios of cytoplasmic/Nuclear TDP-43 were plotted and statistically analyzed using GraphPad for Prism. FIG. 6B; FIG. 9B, right panel.

Fluorescence In Situ Hybridization (FISH)

ES cell-derived MNs were plated on polyornithine/laminin coated coverslips and cultured for 7 days. Coverslips were immersion-fixed for 15 minutes in ice-cold 4% PFA, and washed in 1×PBS. Cells were blocked with 5% normal donkey serum diluted in Tris buffered saline (pH 7.4) with 0.2% Triton X-100 (TBS-T) and incubated in primary antisera (TDP-43 C-term and MAP2) diluted in TBS-T with 5% normal donkey serum overnight at 4° C. After washing with TBS-T, cells were incubated for 1 hour at room temperature with species-specific secondary antibodies coupled to Alexa 488 and 568 (1:1,000; Life Technologies, Carlsbad, Calif., USA). After washing with TBS-T, stained tissue coverslips were mounted on microscope slides in Flouromount (Southern Biotech, Birmingham, Ala., USA) and imaged using a Leica 710LSM confocal microscope at 40× magnification. FIG. 7 and FIG. 8.

Solubility of Mutant TDP-43 Polypeptides

This protocol was adapted from Jo et al. (2014) Nature Communications 5:3496. 500 ul of ice-cold soluble buffer (0.1 M MES (pH 7), 1 mM EDTA, 0.5 mM MgSO4, 1 M sucrose) containing 50 mM N-ethylmaleimide (NEM), 1 mM NaF, 1 mM Na3VO4, 1 mM PMSF and 10 ug/ml each of aprotinin, leupeptin and pepstatin). Cells were by 3-5 passages through a 21-gauge needle, followed by 3-5 passages through a 23-gauge needle. An equivalent volume of homogenate was then collected from each sample and centrifuged at 50,000×g for 20 min at 4° C., and the remainder was stored at −80° C. The supernatant was removed, and each pellet was resuspended in 700 ul RAB buffer (100 mM MES (pH6.8), 10% sucrose, 2 mM EGTA, 0.5 mM MgSO4, 500 mM NaCl, 1 mM MgCl2, 10 mM NaH2PO4, 20 mM NaF) containing 1% N-lauroylsarcosine (Sarkosyl) and protease inhibitors (1 mM PMSF, 50 mM NEM and 10 ug/ml each of aprotinin, leupeptin and pepstatin), vortexed for 1 min at RT, and then incubated at 4° C. overnight with end-over-end rotation. The samples were then centrifuged at 200,000×g for 30 min at 12° C., and the supernatant collected as the sarkosyl-soluble fraction. The pellet was resuspended in 700 ul RAB buffer and passed 3-5 times through a 26-gauge needle to fully disperse the pellet, creating a sarkosyl insoluble fraction. Equivalent portions of sarkosyl soluble and insoluble fractions were then aliquoted and equal volumes of 2×SDS sample buffer was added to each. Samples were heated to 90° C. Equal volumes of each fraction were then loaded onto a 14% SDS gel and electrophoresed for 50 min at 225V followed by western blotting for TDP-43. Densitometry was performed using ImageJ. FIG. 9A. The ratios of soluble:insoluble TDP-43 were plotted and statistically analyzed using GraphPad for Prism. FIG. 9A.

Expression Levels of Mutant TDP-43 Polypeptides

The expression levels of the TDP-43 mutants were analyzed by Western Blot analysis as described herein. Messenger RNA levels in this Example was performed by Quantitative Polymerase Chain Reaction.

Total RNA from each sample was extracted and reverse transcribed using primers that span the junction of normal exon 4 and exon 5 and probe that detect the region of the mouse TDP-43 locus. qPCR of DROSHA was performed using probes and primers of readily available kits.

Specifically, RNA was isolated from embryonic-stem-cell derived motor neurons (ESMN) as described in Example 1.

Total RNA was isolated using Direct-zol RNA Miniprep plus kit according to the manufacturer's protocol (Zymo Research). Total RNA was treated with DNase using Turbo DNA-free kit according to the manufacturer's protocol (Invitrogen) and diluted to 20 ng/μL. Reverse transcription (RT) and PCR were performed in a one-step reaction with Quantitect Probe RT-PCR kit (Qiagen). The qRT-PCR reaction contained 2 μL RNA and 8 μL mixture containing RT-PCR Master mix, ROX dye, RT-mix, and 20× gene specific primer-probe mix to make a final volume of 10 μL.

Unless otherwise noted, final primer and probe concentrations were 0.5 μM and 0.25 μM, respectively. qRT-PCR was performed on a ViiA™ 7 Real-Time PCR Detection System (ThermoFisher). PCR reactions were done in quadruplicates with RT-step at 45° C. 10 min followed by 95° C. 10 min and 2-step cycling 95° C. 5s, 60° C. 30s for 45 cycles in an optical 384-well plate. The sequences of the primers and probes used in the analysis (Pan assay) are provided in Table 2 below.

| | | | |
|---|---|---|---|
| | Forward Primer | Reverse Primer | Probe |
| Assay | (SEQ ID NO) | (SEQ ID NO) | (SEQ ID NO) |

TDP-43

| | | | |
|---|---|---|---|
| Ex3-Ex4 | TGTGACTGTAAACTTCCCAACT | CTCTTCAGCAGTCATGTCCTC | AAGCCCAGACGAGCCTTTGAGAAG |
| | (SEQ ID NO: 11) | (SEQ ID NO: 12) | (SEQ ID NO: 13) |

Stability of Mutant TDP-43 Polypeptides

ES cell colonies were dissociated after 2 days and cultured in ADFNK medium. Medium was replaced at 2 days and supplemented with retinoic acid (100 nM to 2 μM) (Sigma) and Sonic hedgehog (Shh-N; 300 nM) (Curis Inc.) and embryo bodies (EBs) were cultured for 4 days. Day 4 embryoid bodies were treated with cycloheximide (100 μg/ml) to block new protein synthesis. Medium was changed every 4 h with fresh cycloheximide added. Cell lysates were collected at the indicated time points and analyzed by immunoblotting with TDP-43 and GAPDH antibodies. FIG. 9C.

### Example 3: Analysis of TDP-43 Biological Activity by TDP-43 Mutants

Cryptic exons often have GU-rich TDP-43 binding sites, and TDP-43 has been shown to repress recognition of cryptic exons thereby promoting normal splicing. Loss of TDP-43 results in loss of normal mRNA and protein levels of regulated genes. TDP-43 also binds to the 3′ end of its own transcript as a negative feedback autoregulatory loop to maintain TDP-43 levels. The biological activity mutant TDP-43 polypeptides lacking a functional structural domain was tested by evaluating the ability of mutant TDP-43 polypeptides to continue to repress cryptic exon splicing and/or participate in its autoregulatory loop was tested.

ESMNs heterozygous for a wildtype TARDBP gene or a mutated TARDBP gene that encodes a mutant TDP-43 polypeptide lacking a functional RRM1, NLS, or PLD were analyzed for expression products of three genes comprising cryptic exons, the splicing of which is known to be repressed by wildtype TDP-43: Crem, Fyxd2, and Clf1. FIG. 10. Normal spliced Crem, Fyxd2, and Clf1 products were seen in all ESMNs expressing a mutant TDP-43 polypeptide lacking a functional RRM1, NLS, or PLD, and the normal splice products were found at comparable amounts to ESMNs expressing a wildtype TDP-43 polypeptide. FIG. 10. However, the splicing in of cryptic exons was increased in ESMNs expressing a mutant TDP-43 polypeptide lacking a functional RRM1, NLS, or PLD compared to ESMNs expressing a wildtype TDP-43 polypeptide. FIG. 10. This data suggests that mutant TDP-43 polypeptides lacking a functional RRM1, NLS, or PLD fail to repress the cryptic exon splicing of Crem, Fyxd2, and Clf1 genes. FIG. 10.

ESMNs heterozygous for a wildtype TARDBP gene or a mutated TARDBP gene that encodes a mutant TDP-43 polypeptide lacking a functional NLS, RRM1, RRM2, E, or PLD were analyzed for levels of an alternatively spliced TDP-43 mRNA. FIG. 11B. Compared to control ESMNs expressing a wildtype TDP-43 polypeptide, ESMNs expressing a mutant TDP-43 polypeptide lacking a functional NLS, RRM1, E, or PLD exhibited reduced levels of the alternative spliced TDP-43 mRNA. FIG. 11B. ESMNs expressing a mutant TDP-43 polypeptide lacking a functional E exhibited comparable levels of the alternative spliced TDP-43 mRNA. FIG. 11B. This data, combined with the data provided in Example 2 showing that ESMNs expressing TDP-43 mutants lacking a functional NLS or PLD exhibit an ALS phenotype (FIG. 5), suggest that strategies directed toward decreasing the levels of normally spliced TDP-43 mRNA, while sparing the alternatively spliced TDP-43 mRNA, may be therapeutic for TDP-43 associated pathologies.

The materials and methods used to analyze the phenotype of cells expressing a mutant TDP-43 polypeptide lacking a functional structural domain are described below.

Quantitative Polymerase Chain Reaction

Total RNA from each sample was extracted and reverse transcribed using primers that flank splicing regions and probes that detect those regions of interrogated gene locus (Crem, Fxyd2, Clf1, TDP-43). Detectable regions for the interrogated Crem, Fxyd2, and Clf1 genes included those that span the junction of normal and cryptic exon mouse sequences for each interrogated gene. Detectable regions for the interrogated TDP-43 region included those that span an alternative splice region. qPCR of DROSHA was performed using probes and primers of readily available kits.

Specifically, RNA was isolated from embryonic-stem-cell-derived motor neurons (ESMN) differentiated as described in Example 2. Total RNA was isolated using Direct-zol RNA Miniprep plus kit according to the manufacturer's protocol (Zymo Research). Total RNA was treated with DNase using Turbo DNA-free kit according to the manufacturer's protocol (Invitrogen) and diluted to 20 ng/μL. Reverse transcription (RT) and PCR were performed in a one-step reaction with Quantitect Probe RT-PCR kit (Qiagen). The qRT-PCR reaction contained 2 μL RNA and 8 μL mixture containing RT-PCR Master mix, ROX dye, RT-mix, and 20× gene specific primer-probe mix to make a final volume of 10 μL.

Unless otherwise noted, final primer and probe concentrations were 0.5 μM and 0.25 μM, respectively. qRT-PCR was performed on a ViiA™ 7 Real-Time PCR Detection System (ThermoFisher). PCR reactions were done in quadruplicates with RT-step at 45° C. 10 min followed by 95° C. 10 min and 2-step cycling 95° C. 5s, 60° C. 30s for 50 cycles in an optical 384-well plate.

qRT-PCR for evaluating productive Crem splicing from exon 1 to exon 2 of Crem was performed with primers comprising a nucleotide sequence set forth as SEQ ID NO:14 and SEQ ID NO:15, and a primer comprising a nucleotide sequence set forth as SEQ ID NO:16. The splicing of exon 1 to the cryptic exon of Crem was evaluated with primers comprising a nucleotide sequence set forth as SEQ ID NO:17 and SEQ ID NO:18, and a primer comprising a nucleotide sequence set forth as SEQ ID NO:19. The splicing of the cryptic exon of Crem to exon 2 was evaluated with primers comprising a nucleotide sequence set forth as SEQ ID NO:20 and SEQ ID NO:21, and a primer comprising a nucleotide sequence set forth as SEQ ID NO:22.

qRT-PCR for evaluating productive Fyxd2 splicing from exon 3 to exon 4 of Fyxd2 was performed with primers comprising a nucleotide sequence set forth as SEQ ID NO:23 and SEQ ID NO:24, and a primer comprising a nucleotide sequence set forth as SEQ ID NO:25. The splicing of exon 3 to the cryptic exon of Fyxd2 was evaluated with primers comprising a nucleotide sequence set forth as SEQ ID NO:26 and SEQ ID NO:27, and a primer comprising a nucleotide sequence set forth as SEQ ID NO:28. The splicing of the cryptic exon of Fyxd2 to exon 4 was evaluated with primers comprising a nucleotide sequence set forth as SEQ ID NO:29 and SEQ ID NO:30, and a primer comprising a nucleotide sequence set forth as SEQ ID NO:31.

qRT-PCR for productive Crlf1 splice products was performed with primers comprising a nucleotide sequence set forth as SEQ ID NO:32 and SEQ ID NO:33, and a primer comprising a nucleotide sequence set forth as SEQ ID NO:34. The splicing of exon 1 to the cryptic exon of Crlf1 was evaluated with primers comprising a nucleotide sequence set forth as SEQ ID NO:35 and SEQ ID NO:36, and a primer comprising a nucleotide sequence set forth as SEQ ID NO:37. The splicing of the cryptic exon of Crlf1 to exon 2 was evaluated with primers comprising a nucleotide sequence set forth as SEQ ID NO:38 and SEQ ID NO:39, and a primer comprising a nucleotide sequence set forth as SEQ ID NO:40.

Alternatively spliced TDP-43 mRNA lacking a sequence encoding the PLD domain was evaluated using primers comprising a nucleotide sequence set forth as SEQ ID NO:41 and SEQ ID NO:42, and a primer comprising a nucleotide sequence set forth as SEQ ID NO:43.

The sequences of the primers and probes used in each qPCR analysis of this Example (normal and cryptic splicing) are provided in Table 3 below.

TABLE 3

| | | | |
|---|---|---|---|
| | Forward Primer | Reverse Primer | Probe |
| Assay | (SEQ ID NO) | (SEQ ID NO) | (SEQ ID NO) |

Crem

| | | | |
|---|---|---|---|
| Ex1-Ex2 | TGGCTGTAACTGGAGATGAAAC | CCTTGTGGCAAAGCAGTAGTA | ACATGCCAACTTACCAGATCCGAGC |
| | (SEQ ID NO: 14) | (SEQ ID NO: 15) | (SEQ ID NO: 16) |
| Ex1-cryptic | TGGCTGTAACTGGAGATGAAAC | GGAAGAGAAGCAACTCCTCAAA | ACACACACACACACACACACACAC |
| | (SEQ ID NO: 17) | (SEQ ID NO: 18) | (SEQ ID NO: 19) |
| cryptic-Ex2 | CATGGGTTCCAAAGGATCAAAC | TGTGGCAAAGCAGTAGTAGG | ACATGCCAACTTACCAGATCCGAGC |
| | (SEQ ID NO: 20) | (SEQ ID NO: 21) | (SEQ ID NO: 22) |

Fyxd2

| | | | |
|---|---|---|---|
| Ex3-Ex4 | ACTATGAAACCGTCCGCAAA | CCCACAGCGGAACCTTT | CGTGGGCCTCCTCATCATTCTCAG |
| | (SEQ ID NO: 23) | (SEQ ID NO: 24) | (SEQ ID NO: 25) |
| Ex3-cryptic | ACTATGAAACCGTCCGCAAA | CCTCTTTGCTTCACCAAATGTC | CGTGGGCCTCCTCATCATTCTCAG |
| | (SEQ ID NO: 26) | (SEQ ID NO: 27) | (SEQ ID NO: 28) |
| cryptic-Ex4 | TTCTGGAATTCCCACACACTC | CCCACAGCGGAACCTTT | CTCTGAATGAAAGCTGGGCTCTTGGA |
| | (SEQ ID NO: 29) | (SEQ ID NO: 30) | (SEQ ID NO: 31) |

Crlf1

| | | | |
|---|---|---|---|
| Ex1-Ex2 | CTGTCCTCGCTGTGGTC | GGAGGAGCCGATGAGAAG | TCTGTTGCTCTGTGTCCTCGGG |
| | (SEQ ID NO: 32) | (SEQ ID NO: 33) | (SEQ ID NO: 34) |
| Ex1-cryptic | GTCGCCTCTGTTGCTCTG | TCCATCCATTCATCCATCCATC | ACCTCAGTTCCTGGCATATTG |
| | (SEQ ID NO: 35) | (SEQ ID NO: 36) | (SEQ ID NO: 37) |
| cryptic-Ex2 | GAGACCTCAGAGAACTGAATGG | CCAGGTGTGTCTCCATGTATAG | TTCTCATCGGCTCCTCCCTGCAAG |
| | (SEQ ID NO: 38) | (SEQ ID NO: 39) | (SEQ ID NO: 40) |

TDP-43

| | | | |
|---|---|---|---|
| Ex6-Ex7 | GCTGAACCTAAGCATAATAGCAATAG | GGATGAGAAAGCATGTAGACAG | TGGAAGAAGCACTTCATTGAAAGTAGTGC |
| | (SEQ ID NO: 41) | (SEQ ID NO: 42) | (SEQ ID NO: 43) |

### Example 4: Generation of Mice Expressing a Mutated TDP-43 Protein

Although deletion of TDP-43 results in embryonic lethality, embryonic stem cells expressing only a mutant ΔNLS TDP-43 gene or a mutant ΔPLD TDP-43 gene from the endogenous TARDBP locus are viable and may be differentiated into motor neurons in vitro. This data raises the possibility that embryonic stem cells expressing a mutant TDP-43 polypeptide lacking a functional structural domain from an endogenous TARDBP locus may be viable and useful in creating animal models of TDP-43 proteinopathies. For example, such embryonic stem cells may be used to generate non-human animals, e.g., mice, expressing mutant TDP-43 proteins lacking a functional structural domain to examine the role of TDP-43 structural domains in normal and pathological biological processes.

To create embryos or animals that express a mutant TDP-43 protein lacking a functional NLS or PLD domain, the VelociMouse® method (Dechiara, T. M., (2009), Methods Mol Biol 530:311-324; Poueymirou et al. (2007), Nat. Biotechnol. 25:91-99) was used, in which targeted ES cells comprising
(i) at an endogenous TARDBP locus, a TARDBP gene comprising a conditional foxed exon 3 (loxP-Ex3-loxP), a null allele upon Cre-mediated deletion of the foxed exon 3 (-), knockout mutations in the NLS (ΔNLS), a deletion of the prion like domain (ΔPLD), or a wildtype TARDBP gene (WT), see, FIG. 3A, and
(ii) at the other TARDBP locus on a homologous chromosome, a wildtype (WT) TARDBP gene or a null allele upon Cre-mediated deletion of the floxed exon 3 (-) were injected into uncompacted 8-cell stage Swiss Webster embryos. The viability of embryos after fertilization was examined and the ability to produce live-born F0 generation mice was assessed.

Consistent with prior experiments, embryos lacking a functional TDP-43 protein (TDP-43^{-/-}) were not viable and did not survive beyond the E3.5 (FIG. 12) stage. Similarly, embryos expressing only a TDP-43 protein lacking a functional NLS (TDP-43^{ΔNLS/-}) or only a TDP-43 protein lacking a functional PLD (TDP-43^{ΔAPD/-}) were not viable, although such embryos survived longer (FIG. 12). Expression of a wildtype TDP-43 protein from one allele of the TARDPB locus rescued embryos expressing from the other allele on a homologous chromosome either a TDP-43 protein lacking a functional NLS (TDP-43^{ΔNLS/-}) or a TDP-43 protein lacking a functional PLD (TDP-43^{ΔPLD/-}) (FIG. 12).

Live-born F0 generation mice were successfully produced from 8-cell stage Swiss Webster embryos injected with ES cells comprising
(i) at an endogenous TARDBP locus, a wildtype gene (WT), a TARDBP gene comprising cre-mediated deletion of a foxed exon 3 (-), a floxed exon 3 (loxP-Ex3-loxP), knockout mutations in the NLS (ΔNLS), a deletion of the prion like domain (ΔPLD), see, FIG. 3A, and
(iii) at the other TARDBP locus on a homologous chromosome, a wildtype (WT) TARDBP gene.

### Example 4: Phenotypic Analyses of Mice Expressing a Mutated TDP-43 Polypeptide Lacking a Functional Structural Domain

The phenotype of an animal generated in Example 3 was analyzed by evaluating the localization, phosphorylation state, and solubility of TDP-43 polypeptides in spinal cord tissue or motor neurons isolated from the animal. Additionally, the denervation or innervation of the animals' muscles was also determined.

The cytoplasmic and nuclear fractions of motor neurons derived from the spinal cords of 16 week old mice were evaluated by Western Blot analysis with the following: (1) an antibody that recognizes the N-terminus of a wildtype TDP-43 protein and thus binds wildtype TDP-43, ΔNLS TDP-43, and ΔPLD TDP-43, (2) an antibody that recognizes the C-terminus of a wildtype TDP-43 protein and thus binds wildtype TDP-43 and ΔNLS TDP-43, but not ΔPLD TDP-4, or (3) an antibody that recognizes TDP-43 in its phosphorylated form.

As shown in FIGS. 13A-13C, the wildtype and ΔNLS mutant TDP-43 proteins were detected at the expected size of about 43 Kd, while the ΔPLD mutants were detected at the expected size of about 30 Kd. Similar to the ESMNs analyzed in Example 2, mutant TDP-43 polypeptide lacking a functional NLS or PLD redistributed from the nucleus to the cytoplasm in spinal cord tissue, even in the presence of a wildtype TDP-43 protein. FIG. 13A. Phosphorylated TDP-43 polypeptides of about 43 Kd were detected in the cytoplasm of motor neurons derived from the spinal cords of mice expressing mutant ΔNLS or ΔPLD polypeptides, but not of mice expressing only wildtype TDP-43 polypeptides. FIG. 13B. Any phosphorylated TDP-43 in the nucleus of the motor neurons remained undetectable in all samples examined. FIG. 13B. Since the phosphorylation sites are at amino acid positions 409/410, it is not surprising that phosphorylated TDP-43 polypeptides lacking a functional PLD were not detected. FIG. 13B. Motor neurons of spinal cords from 16-week old mice expressing ΔNLS mutant TDP-43 proteins comprising functional mutations in the NLS domain exhibited increased levels insoluble TDP-43 protein overall. FIG. 13C. There did not appear to be an increase in the solubility of TDP-43 proteins in mice expressing ΔPLD mutant TDP-43 proteins. ΔPLD mutants appear soluble, as no ΔPLD mutants were detected in the insoluble fraction. FIG. 13C.

A subset of motor neurons of mice expressing ΔNLS mutant TDP-43 proteins comprising functional mutations in the ΔNLS domain or ΔPLD TDP-43 mutant proteins lacking a functional PLD exhibited extensive cytoplasmic TDP-43 aggregation. FIG. 14. Cytoplasmic aggregation was detected less frequently in the motor neurons of mice expressing ΔPLD mutant proteins compared to those of mice expressing mutant TDP-43 polypeptides lacking a functional NLS. FIG. 14.

Since denervation is one of the first pathological features to manifest in ALS, muscles comprising mostly fast twitch muscles fibers (tibialis anterior) or slow twitch fibers (intercostal muscles) were analyzed for denervation. Mislocalization of TDP-43 resulted in partially innervated endplates (*) and denervation (arrows) of muscles comprising predominantly fast twitch fibers but not slow twitch fibers. FIGS. 15A-15B.

The data shown herein suggests the animals described herein may be valuable disease models of ALS. In typical ALS patients, distal fast-fatigable (FF) motor units are the earliest affected, and neurogenic changes in muscle can be observed before motor neuron loss. Similarly, motor neuron loss in the SOD1 G93A mouse, the most widely used ‘ALS’ model, is also preceded by denervation of skeletal muscles, with early and preferential involvement of FF motor units. In contrast, proximal muscles innervated by predominately slow fibers, such as the intercostal muscles and diaphragm are generally spared until the end—and denervation of these muscles isfatal. Denervation of intercostal muscles may be expected as the disease progresses.

The materials and methods used to analyze the phenotype of mice expressing both (a) a mutant TDP-43 polypeptide lacking a functional NLS or PLD and (b) a wildtype TDP-43 polypeptide are described below.

Intracellular Localization of Mutant TDP-43 Polypeptides and Detection of Phosphorylation

The intracellular localization of TDP-43 mutants was analyzed using an antibody that recognizes the N-terminus of the TDP-43 polypeptide (α-TDP-43 N-term) and an antibody that recognizes the C-terminal prion like domain of the TDP-43 polypeptide (α-TDP-43 C-term) (Proteintech, Rosemont, Ill.). Soluble cytoplasmic protein extracts were prepared by incubating total spinal cord tissue in ice-cold lysis buffer (10 mM KCl, 10 mM Tris-HCl, pH 7.4, 1 mM MgCl2, 1 mM DTT, 0.01% NP-40) supplemented with protease and phosphatase inhibitors (Roche) for 10 minutes on ice. Cells were then passed through a 27-gauge syringe five times. Following centrifugation at 4° C. for 5 minutes at 4000 rpm, the protein supernatant that comprises the soluble cytoplasmic extract was collected. Insoluble nuclear protein extracts were prepared by resuspending the pellet in an equal volume of RBS-100 buffer (10 mMTris-HCl pH 7.4, 2.5 mM MgCl2, 100 mM NaCl, 0.1% NP-40) supplemented with protease and phosphatase. Equal volumes of 2×SDS sample buffer was added to each fraction and samples were heated to 90° C. Equal volumes of each fraction were then loaded onto a 14% SDS gel and electrophoresed for 50 min at 225V followed by western blotting for TDP-43 using the α-TDP-43-N-term antibody (FIG. 13A), the α-TDP-43-C-term antibody (FIG. 13A), or an α-phosphoTDP-43 antibody that detects the phosphorylation of TDP-43 at amino acids 409/410 (FIG. 13B) (Cosmo Bio USA; catalog number CAC-TIP-PTD-M01). Neither the α-TDP-43-C-term antibody nor the α-phosphoTDP-43 antibody would recognize the PLD deletion mutants. Densitometry was performed using ImageJ. (FIGS. 13A and 13B) The ratios of cytoplasmic/Nuclear TDP-43 were plotted and statistically analyzed using GraphPad for Prism. (FIG. 13A, lower panels).

Fluorescence In Situ Hybridization (FISH)

Spinal cords were isolated from the vertebral column, immersion-fixed overnight (or 1 hour for FUS immunostaining) in 4% PFA, and washed in 1×PBS. Spinal cord segments were embedded in 4% low melting point agarose (Promega) and serial transverse sections (70 μm) were cut using a vibratome (Leica VT 1000S) and processed free-floating. Free-floating spinal cord sections were blocked with 5% normal donkey serum diluted in Tris buffered saline (pH 7.4) with 0.2% Triton X-100 (TBS-T) and incubated in primary antisera diluted in TBS-T with 5% normal donkey serum overnight at room temperature. Primary antibodies used are: ChAT (1:250) EMD Millpore Cat AB144P; TDP-43 (1:10,000) Proteintech 10782-2-AP and NeuN (1:500) EMD Millipore MAB377. After washing with TBS-T, tissue sections were incubated for 4 hours at room temperature with species-specific secondary antibodies coupled to Alexa 488, 555, 647 (1:1,000; Life Technologies, Carlsbad, Calif., USA), Cy3 or Cy5 (dilution 1:500; Jackson Immunoresearch Labs, West Grove, Pa., USA). After washing with TBS-T, stained tissue sections were mounted on microscope slides in Flouromount G (Southern Biotech, Birmingham, Ala., USA) and imaged at 40× magnification and 1.5 zoom using an LSM 510 confocal microscope. (FIG. 14)

Solubility of Mutant TDP-43 Polypeptides

This protocol was adapted from Jo et al. (2014) Nature Communications 5:3496. 500 ul of ice-cold soluble buffer (0.1 M MES (pH 7), 1 mM EDTA, 0.5 mM MgSO4, 1 M sucrose) containing 50 mM N-ethylmaleimide (NEM), 1 mM NaF, 1 mM Na3VO4, 1 mM PMSF and 10 ug/ml each of aprotinin, leupeptin and pepstatin). Cells in spinal cord tissue from 16-week old mice were lysed by 3-5 passages through a 21-gauge needle, followed by 3-5 passages through a 23-gauge needle. An equivalent volume of homogenate was then collected from each sample and centrifuged at 50,000×g for 20 min at 4° C., and the remainder was stored at −80° C. The supernatant was removed, and each pellet was resuspended in 700 ul RAB buffer (100 mM MES (pH6.8), 10% sucrose, 2 mM EGTA, 0.5 mM MgSO4, 500 mM NaCl, 1 mM MgCl2, 10 mM NaH2PO4, 20 mM NaF) containing 1% N-lauroylsarcosine (Sarkosyl) and protease inhibitors (1 mM PMSF, 50 mM NEM and 10 ug/ml each of aprotinin, leupeptin and pepstatin), vortexed for 1 min at RT, and then incubated at 4° C. overnight with end-over-end rotation. The samples were then centrifuged at 200,000×g for 30 min at 12° C., and the supernatant collected as the sarkosyl-soluble fraction. The pellet was resuspended in 700 ul RAB buffer and passed 3-5 times through a 26-gauge needle to fully disperse the pellet, creating a sarkosyl insoluble fraction. Equivalent portions of sarkosyl soluble and insoluble fractions were then aliquoted and equal volumes of 2×SDS sample buffer was added to each. Samples were heated to 90° C. Equal volumes of each fraction were then loaded onto a 14% SDS gel and electrophoresed for 50 min at 225V followed by western blotting for TDP-43. Densitometry was performed using ImageJ. (FIG. 13C) The ratios of soluble:insoluble TDP-43 were plotted and statistically analyzed using GraphPad for Prism. (FIG. 13C).

Denervation Studies

For muscle analysis, tibialis anterior (TA), and Intercostal muscles were dissected, post-fixed for 2 hours by immersion in 4% PFA, and washed in 1× phosphate buffered saline, pH 7.4 (PBS). Muscles were then equilibrated in a gradient of sucrose (10%-20%-30% sucrose in 0.1 M phosphate buffer, pH 7.4), embedded in O.C.T. compound (Sakura, Torrance, Calif.) and frozen at −20° C. Consecutive sections (30 μm thick) were cut using a freezing microtome (Leica CM 3050S). Cryosections of muscle (30 μm) were stained with antibodies against Synaptophysin (invitrogen) to identify the pre-synaptic terminal, and Alexa 488-conjugated α-BTX (Invitrogen) to detect post-synaptic acetylcholine receptors. Images were acquired using Zeiss Pascal LSM 510 confocal microscope using a ×10 and ×40 objective. Percentage (%)NMJ innervation was determined by dividing the total number of areas of overlap between VAChT and α-BTX signals (total number innervated endplates) by the number of areas α-BTX signal (total number of endplates).

What is claimed is:

1. A non-human animal cell comprising a mutated TARDBP gene that encodes a mutant TDP-43 polypeptide,
wherein the mutant TDP-43 polypeptide lacks a functional structural domain comprising the nuclear localization signal (NLS), the RNA recognition motif 1 (RRM1), the RNA recognition motif 2 (RRM2), the putative nuclear export signal (E), the prion like domain (PLD), or a combination thereof found in a wildtype TDP-43 polypeptide, and
wherein the non-human animal cell expresses the mutant TDP-43 polypeptide,
optionally wherein the wildtype TDP-43 polypeptide comprises a sequence set forth as SEQ ID NO:1, SEQ ID NO:3, or SEQ ID NO:5.

2. A non-human animal cell comprising
(i) at one chromosome at an endogenous TARDBP locus, a conditional knockout mutation of the TARDBP gene, and
(ii) at the other homologous chromosome at the endogenous TARDBP locus, a deletion of the entire TARDBP coding sequence.

3. A non-human animal tissue comprising the non-human animal cell of claim 1

4. A composition comprising the non-human animal cell of claim 1.

5. A non-human animal comprising a mutated TARDBP gene that encodes a mutant TDP-43 polypeptide,
wherein the mutant TDP-43 polypeptide lacks a functional structural domain comprising the nuclear localization signal (NLS), the RNA recognition motif 1 (RRM1), the RNA recognition motif 2 (RRM2), the putative nuclear export signal (E), the prion like domain (PLD), or a combination thereof found in a wildtype TDP-43 polypeptide, and,
optionally wherein the wildtype TDP-43 polypeptide comprises a sequence set forth as SEQ ID NO:1, SEQ ID NO:3, or SEQ ID NO:5.

6. A non-human animal comprising
i) at one chromosome at an endogenous TARDBP locus, a conditional knockout mutation of the TARDBP gene, and
(ii) at the other homologous chromosome at the endogenous TARDBP locus, a deletion of the entire TARDBP coding sequence.

7. A method of making a non-human animal or a non-human animal cell that expresses a mutant TDP-43 polypeptide comprising modifying the genome of the non-human animal or non-human animal cell to comprise a mutated TARDBP gene that encodes the mutant TDP-43 polypeptide, wherein the mutant TDP-43 polypeptide lacks a functional structural domain compared to a wildtype TDP-43, optionally wherein the wildtype TDP-43 polypeptide comprises a sequence set forth as SEQ ID NO:1, SEQ ID NO:3, or SEQ ID NO:5.

8. A method of identifying a therapeutic candidate for the treatment of a disease, the method comprising
(a) contacting non-human animal cell of claim 1 with the candidate agent,
(b) evaluating the phenotype and/or TDP-43 biological activity of the non-human cell or tissue, and
(c) identifying the candidate agent that restores to the non-human cell or tissue a phenotype and/or TDP-43 biological activity comparable to that of a control cell or tissue that expresses a wildtype TDP-43 polypeptide.

9. A method of identifying a therapeutic candidate for the treatment of a disease, the method comprising
(a) contacting the non-human animal of claim 5 with the candidate agent,
(b) evaluating the phenotype and/or TDP-43 biological activity of the non-human animal, and
(c) identifying the candidate agent that restores to the non-human a phenotype and/or TDP-43 biological activity.

10. A method of evaluating the biological function of a TDP-43 structural domain comprising
(a) modifying an embryonic stem (ES) cell to comprise a mutated TARDBP gene that encodes a mutant TDP-43 polypeptide that lacks a functional structural domain selected from the group consisting of the nuclear localization signal (NLS), the first RNA recognition motif (RRM1), the first RNA recognition motif (RRM2), the putative nuclear export signal (E), the prion like domain (PLD), and a combination thereof,
(b) optionally differentiating the modified ES cell in vitro and/or obtaining a genetically modified non-human animal from the modified ES cell, and
(c) evaluating the phenotype and/or TDP-43 biological activity of the genetically modified ES cell, primitive ectoderm derived therefrom, motor neurons derived therefrom, or a non-human animal derived therefrom.

11. An anti sense oligonucleotide comprising a gapmer motif targeting a TDP-43 mRNA sequence that encodes a PLD of a TDP-43 polypeptide and/or comprises untranslated sequences downstream of exon 6 and upstream of exon 7.

12. An siRNA comprising a sequence targeting a TDP-43 mRNA sequence that encodes a PLD of a TDP-43 polypeptide and/or comprises untranslated sequences downstream of exon 6 and upstream of exon 7.

13. A CRISPR/Cas system comprising a Cas9 protein and at least one gRNA, wherein the gRNA recognizes a sequence at or near sequences encoding for alternative splice sites that result in alternative mRNA that encode a truncated TDP-43 polypeptide lacking a PLD.

14. A mutant TDP-43 polypeptide comprising a sequence set forth as SEQ ID NO:1, 3, or 5 modified to comprise to one or more of the following:
(a) a point mutation of an amino acid in the NLS,
(b) a point mutation of an amino acid in the RRM1,
(c) a point mutation of an amino acid in the RRM2,
(d) a deletion of at least a portion of the nuclear export signal, and
(e) a deletion of at least a portion of the prion-like domain.

15. A nucleic acid comprising a nucleic acid sequence encoding the mutant TDP-43 polypeptide of claim 14.

16. A method of selectively decreasing TDP-43 mRNA that encode a TDP-43 polypeptide comprising a PLD while sparing alternative TDP-43 mRNA that encode a truncated TDP-43 lacking a PLD in a cell, the method comprising introducing into the cell:
(i) an antisense oligonucleotide comprising a gapmer motif targeting a TDP-43 mRNA sequence that encodes a PLD of a TDP-43 polypeptide and/or comprises untranslated sequences downstream of exon 6 and upstream of exon 7,
(ii) an siRNA comprising a sequence targeting a TDP-43 mRNA sequence that encodes a PLD of a TDP-43 polypeptide and/or comprises untranslated sequences downstream of exon 6 and upstream of exon 7, and/or
(iii) a CRISPR/Cas system comprising a Cas9 protein and at least one gRNA, wherein the gRNA recognizes a sequence at or near sequences encoding for alternative splice sites that result in alternative mRNA that encode a truncated TDP-43 polypeptide lacking a PLD.
